(19) United States
(12) Patent Application Publication
(10) Pub. No.: US 2019/0016771 A1
(43) Pub. Date: Jan. 17, 2019

(54) TRIMERIC COSTIMULATORY TNF FAMILY LIGAND-CONTAINING ANTIGEN BINDING MOLECULES

(71) Applicant: Hoffmann-La Roche Inc., Little Falls, NJ (US)

(72) Inventors: Maria AMANN, Bogen (DE); Peter Bruenker, Hittnau (CH); Christina Claus, Bern (CH); Claudia Ferrara Koller, Zug (CH); Sandra Grau-Richards, Birmensdorf (CH); Christian Klein, Bonstetten (CH); Viktor Levitski, Birmensdorf (CH); Ekkehard Moessner, Kreuzlingen (CH); Pablo Umana, Wollerau (CH)

(73) Assignee: Hoffmann-La Roche Inc., Little Falls, NJ (US)

(21) Appl. No.: 16/144,687

(22) Filed: Sep. 27, 2018

Related U.S. Application Data
(63) Continuation of application No. PCT/EP2017/057143, filed on Mar. 27, 2017.

(30) Foreign Application Priority Data
Mar. 29, 2016 (EP) 16162539.7

(51) Int. Cl. C07K 14/525; C12N 15/62; C07K 16/24; C07K 14/78; A61P 35/00; C07K 16/46

(52) U.S. Cl. CPC C07K 14/525; C12N 15/62; C07K 16/241; A61K 38/191; A61P 35/00; C07K 16/46; C07K 14/78

(57) ABSTRACT

The invention relates to novel trimeric costimulatory TNF family ligand-containing antigen binding molecules comprising three fusion polypeptides, each of the three fusion polypeptides comprising (a) an ectodomain of a costimulatory TNF family ligand selected from the group consisting of 4-1BBL, OX40L and GITRL or fragments thereof, (b) a trimerization domain, in particular a trimerization domain derived from human cartilage matrix protein (huCMP) of SEQ ID NO:1, and (c) a moiety capable of specific binding to a target cell antigen, and to methods of producing these molecules and to methods of using the same.

### FIELD OF THE INVENTION

The invention relates to novel trimeric costimulatory TNF family ligand-containing antigen binding molecules comprising three fusion polypeptides, each of the three fusion polypeptides comprising (a) an ectodomain of a TNF ligand family member or fragments thereof, (b) a trimerization domain, in particular a trimerization domain derived from human cartilage matrix protein (hCMP, SEQ ID NO:1), and (c) a moiety capable of specific binding to a target cell antigen, and to methods of producing these molecules and to methods of using the same. The invention further relates to methods of producing these molecules and to methods of using the same.

### BACKGROUND

Ligands interacting with molecules of the TNF (tumor necrosis factor) receptor superfamily have pivotal roles in the organization and function of the immune system. While regulating normal functions such as immune responses, hematopoiesis and morphogenesis, the TNF family ligands (also called cytokines) play a role in tumorgenesis, transplant rejection, septic shock, viral replication, bone resorption, rheumatoid arthritis and diabetes (Aggarwal, 2003). The TNF ligand family comprises 18 genes encoding 19 type II (i.e. intracellular N terminus and extracellular C-terminus) transmembrane proteins, characterized by the presence of a conserved C-terminal domain coined the ‘TNF homology domain’ (THD). This domain is responsible for receptor binding and is thus critical for the biological activity of the TNF ligand family members. The sequence identity between family members is ˜20-30% (Bodmer, 2002). Members of the TNF ligand family exert their biological function as self-assembling, noncovalent trimers (Banner et al, 1993). Thus, the TNF family ligands need to form a trimer that is able to bind to and to activate the corresponding receptors of TNFR superfamily.

Several approaches have been described to generate artificial multimers with defined stoichiometries of recombinant antibodies or other proteins. WO 01/49866 discloses recombinant fusion proteins comprising a TNF cytokine and a multimerization component, particularly proteins from the C1q protein family such as ACRP30 or a collectin. A disadvantage of these fusion proteins is, however, that the trimerization domain usually has a large molecular weight and/or that the trimerization is rather inefficient. WO 2007/014744, WO 2009/000538 and Wyzgol et al. (2009) disclose fusion proteins comprising a TNF cytokine and a trimerization domain from the chicken protein tenascin. Biological activity could be strongly enhanced. However, a trimerization domain that is derived from a non-human origin, could have the disadvantage of causing immunogenicity reactions in the human body. Human cartilage matix protein (huCMP) is a major extracellular matrix protein localized specifically in cartilage. Its C-terminal region forms a three-stranded alpha-helical coiled-coil structure (Beck, 1996).

WO 2006/121810 relates to a trimeric OX40 ligand fusion polypeptide including a ligand domain, a trimerization domain and an immunoglobulin Fc domain (single chain CH2, CH3 and hinge). Upon assembly of the OX-40L fusion polypeptide into a trimer, two Fc domains dimerize and the third, unpaired Fc domain associates with an unpaired Fc domain of a second OX-40L fusion protein trimer so that a stable OX40L fusion polypeptide hexamer is provided. The trimerization domain is an isoleucine zipper domain derived from yeast GCN4. A similar hexamer comprising a human TRAF2 trimerization domain instead of the isoleucine zipper domain is disclosed in WO 2015/183902. OX40L fusion proteins with different types of trimerization domains are also described in AU 2013/263717 B2.

Some members of the TNF ligand family have costimulatory effects on T-cells, meaning that they sustain T-cell responses after initial T cell activation (Watts, 2005). 4-1BBL, OX40L, GITRL, CD70, CD30L and LIGHT belong to this group of costimulatory TNF family ligands.

Among several costimulatory molecules, the tumor necrosis factor (TNF) receptor family member OX40 (CD134) plays a key role in the survival and homeostasis of effector and memory T cells. OX40 (CD134) regulates immune responses against infections, tumors and self-antigens and its expression has been demonstrated on the surface of T-, NKT-, NK-cells as well as neutrophils and shown to be strictly inducible or strongly upregulated in response to various stimulatory signals. Combined with T-cell receptor triggering, OX40 engagement on T-cells by its natural ligand or agonistic antibodies leads to synergistic activation of the PI3K and NFκB signalling pathways. In turn, this results in enhanced proliferation, increased cytokine receptor and cytokine production and better survival of activated T-cells.

4-1BB (CD137), a member of the TNF receptor superfamily, has been first identified as a molecule whose expression is induced by T-cell activation (Kwon and Weissman, 1989). Subsequent studies demonstrated expression of 4-1BB in T- and B-lymphocytes (Snell et al., 2011; Zhang et al., 2010), NK-cells (Lin et al., 2008), NKT-cells (Kim et al., 2008), monocytes (Kienzle and von Kempis, 2000; Schwarz et al., 1995), neutrophils (Heinisch et al., 2000), mast (Nishimoto et al., 2005) and dendritic cells as well as cells of non-hematopoietic origin such as endothelial and smooth muscle cells (Broll et al., 2001; Olofsson et al., 2008). Expression of 4-1BB in different cell types is mostly inducible and driven by various stimulatory signals, such as T-cell receptor (TCR) or B-cell receptor triggering, as well as signaling induced through co-stimulatory molecules or receptors of pro-inflammatory cytokines (Diehl et al., 2002; von Kempis et al., 1997; Zhang et al., 2010).

Expression of 4-1BB ligand (4-1BBL or CD137L) is more restricted and is observed on professional antigen presenting cells (APC) such as B-cells, dendritic cells (DCs) and macrophages. Inducible expression of 4-1BBL is characteristic for T-cells, including both αβ and γδ T-cell subsets, and endothelial cells (reviewed in Shao and Schwarz, 2011).

CD137 signaling is known to stimulate IFNγ secretion and proliferation of NK cells (Buechele et al., 2012; Lin et al., 2008; Melero et al., 1998) as well as to promote DC activation as indicated by their increased survival and capacity to secret cytokines and upregulate co-stimulatory molecules (Choi et al., 2009; Futagawa et al., 2002; Wilcox et al., 2002). However, CD137 is best characterized as a co-stimulatory molecule which modulates TCR-induced activation in both the CD4+ and CD8+ subsets of T-cells. In combination with TCR triggering, agonistic 4-1BB-specific antibodies enhance proliferation of T-cells, stimulate lymphokine secretion and decrease sensitivity of T-lymphocytes to activation-induced cells death (reviewed in Snell et al., 2011).

In line with these co-stimulatory effects of 4-1BB antibodies on T-cells in vitro, their administration to tumor bearing mice leads to potent anti-tumor effects in many experimental tumor models (Melero et al., 1997; Narazaki et al., 2010). However, 4-1BB usually exhibits its potency as an anti-tumor agent only when administered in combination with other immunomodulatory compounds (Curran et al., 2011; Guo et al., 2013; Morales-Kastresana et al., 2013; Teng et al., 2009; Wei et al., 2013), chemotherapeutic reagents (Ju et al., 2008; Kim et al., 2009), tumor-specific vaccination (Cuadros et al., 2005; Lee et al., 2011) or radiotherapy (Shi and Siemann, 2006). In vivo depletion experiments demonstrated that CD8+ T-cells play the most critical role in anti-tumoral effect of 4-1BB-specific antibodies. However, depending on the tumor model or combination therapy, which includes anti-4-1BB, contributions of other types of cells such as DCs, NK-cells or CD4+ T-cells have been reported (Melero et al., 1997; Murillo et al., 2009; Narazaki et al., 2010; Stagg et al., 2011).

In addition to their direct effects on different lymphocyte subsets, 4-1BB agonists can also induce infiltration and retention of activated T-cells in the tumor through 4-1BB-mediated upregulation of intercellular adhesion molecule 1 (ICAM1) and vascular cell adhesion molecule 1 (VCAM1) on tumor vascular endothelium (Palazon et al., 2011). 4-1BB triggering may also reverse the state of T-cell anergy induced by exposure to soluble antigen that may contribute to disruption of immunological tolerance in the tumor micro-environment or during chronic infections (Wilcox et al., 2004).

It appears that the immunomodulatory properties of 4-1BB agonistic antibodies in vivo require the presence of the wild type Fc-portion on the antibody molecule thereby implicating Fc-receptor binding as an important event required for the pharmacological activity of such reagents as has been described for agonistic antibodies specific to other apoptosis-inducing or immunomodulatory members of the TNFR-superfamily (Li and Ravetch, 2011; Teng et al., 2009). However, systemic administration of 4-1BB-specific agonistic antibodies with the functionally active Fc domain also induces expansion of CD8⁺ T-cells associated with liver toxicity (Dubrot et al., 2010) that is diminished or significantly ameliorated in the absence of functional Fc-receptors in mice. In human clinical trials (ClinicalTrials.gov, NCT00309023), Fc-competent 4-1BB agonistic antibodies (BMS-663513) administered once every three weeks for 12 weeks induced stabilization of the disease in patients with melanoma, ovarian or renal cell carcinoma. However, the same antibody given in another trial (NCT00612664) caused grade 4 hepatitis leading to termination of the trial (Simeone and Ascierto, 2012).

Collectively, the available pre-clinical and clinical data clearly demonstrate that there is a high clinical need for effective 4-1BB agonists. However, new generation drug candidates should not only effectively engage 4-1BB on the surface of hematopoietic and endothelial cells but also be capable of achieving that through mechanisms other than binding to Fc-receptors in order to avoid uncontrollable side effects. The latter may be accomplished through preferential binding to and oligomerization on tumor-specific or tumor-associated moieties. WO 2011/109789 discloses fusion proteins of a TNF ligand and a targeting moiety, however these molecules do not comprise a trimerization domain.

The novel trimeric antigen binding molecules of this invention consist of fusion polypeptides combining a costimulatory TNF ligand and a moiety capable of specific binding to a tumor-specific target cell antigen and are stable as the fusion polypeptides are covalently linked to each other by a huCMP trimerization domain. The stable TNF family ligand trimers are able to trigger TNF receptors not only effectively, but also very selectively at the desired site based on the fact that they comprise three moieties capable of specific binding to a target cell antigen. Side effects may therefore be drastically reduced.

### SUMMARY OF THE INVENTION

The invention relates to a trimeric costimulatory TNF family ligand-containing antigen binding molecule comprising three fusion polypeptides, each of the three fusion polypeptides comprising

(a) an ectodomain of a costimulatory TNF family ligand selected from the group consisting of 4-1BBL, OX40L and GITRL or fragments thereof,
(b) a trimerization domain derived from human cartilage matrix protein (huCMP) of amino acid sequence of SEQ ID NO:1, and
(c) a moiety capable of specific binding to a target cell antigen.

In one aspect, the trimeric costimulatory TNF family ligand-containing antigen binding molecule comprises three fusion polypeptides, each of the three fusion polypeptides comprising a trimerization domain comprising an amino acid sequence having at least 95% identity to SEQ ID NO:2. More particularly, the trimerization domain comprises the amino acid sequence of SEQ ID NO:2.

In particular, the costimulatory TNF family ligand is selected from the group consisting of 4-1BBL, OX40L and GITRL, more particularly the costimulatory TNF family ligand member is selected from 4-1BBL and OX40L.

In one aspect, the costimulatory TNF family ligand is 4-1BBL. In particular, the trimeric costimulatory TNF family ligand-containing antigen binding molecule comprises three fusion polypeptides, wherein in each of the three fusion polypeptides comprises the ectodomain of a TNF family ligand comprising the amino acid sequence selected from the group consisting of SEQ ID NO:3, SEQ ID NO:4, SEQ ID NO:5, SEQ ID NO:6, SEQ ID NO:7, SEQ ID NO:8, SEQ ID NO:9 and SEQ ID NO:10, particularly the amino acid sequence of SEQ ID NO:3 or SEQ ID NO:7. In a further aspect, the trimeric costimulatory TNF family ligand-containing antigen binding molecule comprises three fusion polypeptides, each of the three fusion polypeptides comprising (a) an ectodomain of a TNF family ligand comprising the amino acid sequence selected from the group consisting of SEQ ID NO:3, SEQ ID NO:4, SEQ ID NO:5, SEQ ID NO:6, SEQ ID NO:7, SEQ ID NO:8, SEQ ID NO:9 and SEQ ID NO:10, (b) a trimerization domain comprising the amino acid sequence of SEQ ID NO:2 and (c) a moiety capable of specific binding to a target cell antigen.

In another aspect, the costimulatory TNF family ligand is OX40L. In particular, the trimeric costimulatory TNF family ligand-containing antigen binding molecule comprises three fusion polypeptides, wherein in each of the three fusion polypeptides the ectodomain of a TNF family ligand comprises the amino acid sequence selected from the group consisting of SEQ ID NO:11, SEQ ID NO:12 and SEQ ID NO:13. In a further aspect, the trimeric costimulatory TNF family ligand-containing antigen binding molecule comprises three fusion polypeptides, wherein each of the three fusion polypeptides comprises (a) an ectodomain of a TNF family ligand comprising the amino acid sequence selected from the group consisting of SEQ ID NO:11, SEQ ID NO:12 and SEQ ID NO:13, (b) a trimerization domain comprising the amino acid sequence of SEQ ID NO:2 and (c) a moiety capable of specific binding to a target cell antigen.

In another aspect, provided is a trimeric costimulatory TNF family ligand-containing antigen binding molecule comprising three fusion polypeptides as described before, wherein the ectodomain of a TNF ligand family member or a fragment thereof is fused at the N-terminal amino acid to the C-terminal amino acid of the trimerization domain, optionally through a peptide linker.

In a further aspect, provided is a trimeric costimulatory TNF family ligand-containing antigen binding molecule comprising three fusion polypeptides as described before, wherein the moiety capable of specific binding to a target cell antigen is fused at the C-terminal amino acid to the N-terminal amino acid of the trimerization domain, optionally through a peptide linker.

In another aspect, the invention provides a trimeric costimulatory TNF family ligand-containing antigen binding molecule comprising three fusion polypeptides as described before, wherein the moiety capable of specific binding to a target cell antigen is selected from the group consisting of an antibody fragment, a Fab molecule, a crossover Fab molecule, a single chain Fab molecule, a Fv molecule, a scFv molecule, a single domain antibody, an aVH and a scaffold antigen binding protein. In a particular aspect, the moiety capable of specific binding to a target cell antigen is a Fab molecule capable of specific binding to a target cell antigen. In a further aspect, said Fab molecule is fused at the C-terminal amino acid of the CH1 domain to the N-terminal amino acid of the trimerization domain, optionally through a peptide linker.

In a further aspect, provided is a trimeric costimulatory TNF family ligand-containing antigen binding molecule comprising three fusion polypeptides as described before, wherein the target cell antigen is selected from the group consisting of Fibroblast Activation Protein (FAP), Melanoma-associated Chondroitin Sulfate Proteoglycan (MCSP), Epidermal Growth Factor Receptor (EGFR), Carcinoembryonic Antigen (CEA), CD19, CD20 and CD33. In a particular aspect, the target cell antigen is Fibroblast Activation Protein (FAP).

In a further aspect, provided is a trimeric costimulatory TNF family ligand-containing antigen binding molecule comprising three fusion polypeptides as described before, wherein the moiety capable of specific binding to FAP comprises

(a) a VH domain comprising (i) CDR-H1 comprising the amino acid sequence of SEQ ID NO:14, (ii) CDR-H2 comprising the amino acid sequence of SEQ ID NO:15 and (iii) CDR-H3 comprising the amino acid sequence of SEQ ID NO:16, and a VL domain comprising (iv) CDR-L1 comprising the amino acid sequence of SEQ ID NO:17, (v) CDR-L2 comprising the amino acid sequence of SEQ ID NO:18 and (vi) CDR-L3 comprising the amino acid sequence of SEQ ID NO:19 or
(b) a VH domain comprising (i) CDR-H1 comprising the amino acid sequence of SEQ ID NO:20, (ii) CDR-H2 comprising the amino acid sequence of SEQ ID NO:21 and (iii) CDR-H3 comprising the amino acid sequence of SEQ ID NO:22, and a VL domain comprising (iv) CDR-L1 comprising the amino acid sequence of SEQ ID NO:23, (v) CDR-L2 comprising the amino acid sequence of SEQ ID NO:24 and (vi) CDR-L3 comprising the amino acid sequence of SEQ ID NO:25.

In a further aspect, the moiety capable of specific binding to FAP comprises

(a) a VH domain comprising the amino acid sequence of SEQ ID NO:26 and a VL domain comprising the amino acid sequence of SEQ ID NO:27, or (b) a VH domain comprising the amino acid sequence of SEQ ID NO:28 and a VL domain comprising the amino acid sequence of SEQ ID NO:29.

In one aspect, provided is a trimeric costimulatory TNF family ligand-containing antigen binding molecule comprising three fusion polypeptides as described before, wherein said three fusion polypeptides are identical.

In another aspect, the invention provides a fusion polypeptide comprising (a) an ectodomain of a costimulatory TNF ligand family member or a fragment thereof, (b) a trimerization domain derived from human cartilage matrix protein (huCMP) of the amino acid sequence of SEQ ID NO:2, wherein said trimerization domain is capable of mediating stable association of said fusion polypeptide with two further such fusion polypeptides and (c) a moiety capable of specific binding to a target cell antigen. In a particular aspect, the moiety of specific binding to a target cell antigen is a VH domain or a VL domain capable of specific binding to a target cell antigen.

In yet a further aspect, provided is a fusion polypeptide, wherein the fusion polypeptide comprises

(a) an ectodomain of a TNF ligand family comprising the amino acid sequence selected from the group consisting of SEQ ID NO:3, SEQ ID NO:4, SEQ ID NO:5, SEQ ID NO:6, SEQ ID NO:7, SEQ ID NO:8, SEQ ID NO:9 and SEQ ID NO:10,
(b) a trimerization domain comprising the amino acid sequence of SEQ ID NO:2 and
(c) a VH domain comprising (i) CDR-H1 comprising the amino acid sequence of SEQ ID NO:14, (ii) CDR-H2 comprising the amino acid sequence of SEQ ID NO:15 and (iii) CDR-H3 comprising the amino acid sequence of SEQ ID NO:16 or a VH domain comprising (i) CDR-H1 comprising the amino acid sequence of SEQ ID NO:20, (ii) CDR-H2 comprising the amino acid sequence of SEQ ID NO:21 and (iii) CDR-H3 comprising the amino acid sequence of SEQ ID NO:22.

According to another aspect of the invention, there is provided an isolated polynucleotide encoding a trimeric costimulatory TNF family ligand-containing antigen binding molecule as defined herein before or a fusion polypeptide as described herein before. The invention further provides a vector, particularly an expression vector, comprising the isolated polynucleotide of the invention and a host cell comprising the isolated polynucleotide or the vector of the invention. In some embodiments the host cell is a eukaryotic cell, particularly a mammalian cell.

In another aspect, provided is a method for producing the trimeric costimulatory TNF family ligand-containing antigen binding molecule of the invention, comprising the steps of (i) culturing the host cell of the invention under conditions suitable for expression of said antigen binding molecule, and (ii) isolating said trimeric antigen binding molecule. The invention also encompasses a trimeric costimulatory TNF family ligand-containing antigen binding molecule produced by the method of the invention.

The invention further provides a pharmaceutical composition comprising trimeric costimulatory TNF family ligand-containing antigen binding molecule of the invention and at least one pharmaceutically acceptable excipient.

Also encompassed by the invention is the trimeric costimulatory TNF family ligand-containing antigen binding molecule of the invention, or the pharmaceutical composition of the invention, for use as medicament. In one aspect, provided is a trimeric costimulatory TNF family ligand-containing antigen binding molecule of the invention, or the pharmaceutical composition of the invention, for use in the treatment of a disease in an individual in need thereof. In a specific embodiment, provided is the trimeric costimulatory TNF family ligand-containing antigen binding molecule of the invention, or the pharmaceutical composition of the invention, for use in the treatment of cancer.

Also provided is the use of the trimeric costimulatory TNF family ligand-containing antigen binding molecule of the invention for the manufacture of a medicament for the treatment of a disease in an individual in need thereof, in particular for the manufacture of a medicament for the treatment of cancer, as well as a method of treating a disease in an individual, comprising administering to said individual a therapeutically effective amount of a composition comprising the trimeric costimulatory TNF family ligand-containing antigen binding molecule of the invention in a pharmaceutically acceptable form. In a specific embodiment, the disease is cancer. In any of the above embodiments the individual is preferably a mammal, particularly a human.

### BRIEF DESCRIPTION OF THE DRAWINGS

FIG. 1A shows the chain encoding a FAP-targeted 4-1BB ligand-containing fusion polypeptide (VH-CH1 of a Fab molecule capable of specific binding to FAP) and the light chain (VL-CL with FAP specificity). Disulfide bonds are formed between three FAP-targeted 4-1BB ligand-containing fusion poylpeptide chains leading to the formation of covalently linked CMP-trimeric 4-1BB ligand-containing antigen binding molecules, targeted to FAP. FIG. 1B shows the trimeric 4-1BBL-containing antigen binding molecule comprising three Fabs capable of specific binding to FAP (FAP-targeted). FIG. 1C is a drawing of the monomeric 4-1BB Fc(kih) molecules as prepared in Example 3.

FIGS. 2A to 2F show the binding of the trimeric FAP-targeted-CMP-4-1BB ligand-containing antigen binding molecule to recombinant 4-1BB Fc (kih) receptor as assessed by surface plasmon resonance. In FIG. 2A binding to human 4-1BB Fc (kih) and in FIG. 2B the setup of the assay is shown, 4-1BB Fc (kih) is immobilized on a CM5 chip. FIG. 2C shows binding to cynomolgus 4-1BB Fc (kih), the setup of the assay is illustrated in FIG. 2D. In FIG. 2E the binding to murine 4-1BB Fc (kih) and in FIG. 2F the assay setup is shown.

FIGS. 3A to 3C illustrate the binding of recombinant 4-1BB Fc(kih) to FAP-targeted-CMP-4-1BB ligand-containing antigen binding molecule. In FIG. 3A the setup of the affinity measurement is shown. FIG. 3B shows the binding of human 4-1BB Fc(kih) to the FAP-targeted-CMP-4-1BB ligand-containing antigen binding molecule and FIG. 3C shows the binding of cynomolgus 4-1BB Fc(kih) to FAP-targeted-CMP-4-1BB ligand-containing antigen binding molecule.

FIGS. 4A to 4D relate to the binding of FAP-targeted CMP trimeric 4-1BB ligand-containing antigen binding molecules (filled circles) or DP47-targeted CMP trimeric 4-1BB ligand-containing antigen binding molecules (open circles) or DP47 hu IgG P329G control (filled triangle) to resting (naïve) CD8⁺ and CD4⁺ T cells shown in the upper panels (FIGS. 4A and 4B) or activated CD8⁺ and CD4⁺ T cells shown in the lower panels (FIGS. 4C and 4D). Shown is the binding as median of fluorescence intensity (MFI) of red macrophytic algae Phycoerythrin (R-PE)-labeled anti-human IgG F(ab′)2-specific goat IgG F(ab′)2 fragment which is used as secondary detection antibody versus the concentration of FAP- or DP47-targeted CMP-trimeric 4-1BB ligand-containing antigen binding molecules. MFI was measured by flow cytometry and baseline corrected by subtracting the MFI of the blank control.

FIGS. 5A to 5C show the binding of FAP-targeted CMP-trimeric 4-1BB ligand-containing antigen binding molecules (filled circles) or DP47-targeted CMP-trimeric 4-1BB ligand-containing antigen binding molecules (open circles) to human fibroblast activation protein (FAP)-expressing transfected mouse embryonic fibroblast cell line 3T3-huFAP clone 39. The binding is characterized by plotting the MFI of polyclonal FITC-labeled anti-human IgG H+L-specific goat IgG F(ab′)2 fragment (FIG. 5A) or the MFI of FITC-labeled monoclonal anti-human IgG κ-light chain-specific mouse IgG1 κ (clone G18-145) (FIG. 5B) or the MFI of PE-labeled anti-human 4-1BBL mouse IgG2b (FIG. 5C) which are used as secondary detection antibodies versus the concentration in nM of tested CMP-trimeric 4-1BB ligand constructs. MFI was measured by flow cytometry and baseline corrected by subtracting the MFI of the blank control.

The scheme in FIG. 6 illustrates the general principal of the NF-κB activity assay described in Example 5.1.2 using a reporter cell line. The ratio of FAP-expressing tumor cells to the reporter cell line HeLa-huCD137-NF-κB-luciferase was 5 to 1.

FIG. 7A to 7C show the activation of the NF-κB signaling pathway by FAP-targeted CMP-trimeric 4-1BB ligand-containing antigen binding molecule (FAP-CMP-4-1BBL, filled circles) is strictly dependent on its binding to FAP-expressing target cells and an optimal concentration inducing maximal crosslinking (bell shape curve). NF-κB reporter HeLa cells were co-cultured with the indicated tumor cells (3T3-huFAP clone 39 in FIG. 7A, MV-3 in FIG. 7B and WM-266-4 in FIG. 7C) exhibiting different levels of cell surface FAP expression. Luciferase activity was assessed as described in Example 5.1.2 after culturing cells in the absence or presence of 4-1BBL-containing antigen binding molecules at the indicated concentrations. Open circles refer to DP47 untargeted CMP-trimeric 4-1BB ligand-containing antigen binding molecule. Activity is characterized by blotting the units of released light (URL) measured during 0.5 s versus the concentration in nM of tested constructs. URL are emitted due to luciferase-mediated oxidation of luciferin to oxyluciferin.

The scheme in FIG. 8 illustrates the general principal of the antigen-specific CD8⁺ T cell activation assay as described in Example 5.1.3 using NLV-specific CD8⁺ T cells. The ratio of FAP-expres sing tumor cells to NLV-specific CD8 T cells was 8 to 1.

FIGS. 9A to 9F show the prolonged IFNγ secretion and CD137 (4-1BB) expression of NLV-specific CD8⁺ T cells is strictly dependent on simultaneous activation of T-cells via recognition of NLV-HLA-A2 complexes (signal 1) and 4-1BB-triggering by FAP-targeted huCMP trimeric 4-1BB ligand-containing antigen binding molecules (signal 2). Filled circles symbolize FAP-targeted CMP-trimeric 4-1BB ligand-containing antigen binding molecules; open circles symbolize DP47-targeted huCMP-trimeric 4-1BB ligand-containing antigen binding molecules. The effect of 4-1BB upregulation is shown in graphs of FIGS. 9A (no activation with NLV-peptide), 9B (activation with NLV peptide in a concentration of 10⁻⁹ M) and 9C (activation with NLV peptide in a concentration of 10⁻⁸ M), whereas the effect of INFγ expression of CD8⁺ T cells is presented in graphs of FIGS. 9D (no activation with NLV-peptide), 9E (activation with NLV peptide in a concentration of 10⁻⁹ M) and 9F (activation with NLV peptide in a concentration of 10⁻⁸ M). Shown is always the frequency in percentage of positive cells in the total CD8⁺ T cell population. All T cell activation curves are bell-shaped and show that the co-stimulation via FAP-targeted huCMP-trimeric 4-1BB ligand-containing antigen binding molecule is restricted to a defined concentration window of around 0.6 nM.

FIG. 10 shows the binding of FAP-targeted huCMP trimeric OX40 ligand-containing antigen binding molecules to FAP positive WM-266-4 cells. WM-266-4 cells express high levels of human fibroblast activation protein (huFAP). Only FAP-targeted huCMP trimeric OX40 ligand-containing antigen binding molecule (filled circle) but not the untargeted DP47 huIgG P329G control antibody (Control F, filled diamond) bound to WM-266-4 cells. Shown is the binding as median of fluorescence intensity (MFI) of FITC labeled anti-human IgG F(ab′)2 specific goat IgG F(ab′)2 fragment which is used as secondary detection antibody. MFI was measured by flow cytometry. The x-axis shows the concentration of antigen binding molecules.

FIGS. 11A and 11B show the binding of FAP-targeted huCMP trimeric OX40 ligand-containing antigen binding molecules to resting and activated human CD4⁺ T cells, respectively. OX40 is not expressed on resting human CD4⁺ T cells. In the absence of human OX40 expressing cells no binding was observed (FIG. 11A). After activation of human PBMCs OX40 is up-regulated on CD4⁺ T cells (FIG. 11B). FAP-targeted huCMP trimeric OX40 ligand-containing antigen binding molecules bound to OX40⁺ activated CD4⁺ T cells. Shown is the binding as median of fluorescence intensity (MFI) of FITC labeled anti-human IgG F(ab′)₂ specific goat IgG F(ab′)2 fragment which is used as secondary detection antibody. MFI was measured by flow cytometry and baseline corrected by subtracting the MFI of the blank control. The x-axis shows the concentration of antigen binding molecules. In FIGS. 11C and 11D is shown the binding of FAP-targeted huCMP trimeric OX40 ligand-containing antigen binding molecules to resting and activated human CD8⁺ T cells, respectively. OX40 is not expressed on resting human CD8⁺ T cells. In the absence of human OX40 expressing cells no binding was observed (FIG. 11C). After activation of human PBMCs OX40 is up-regulated on CD8⁺ T cells (FIG. 11D). OX40 expression on human CD8⁺ T cells is lower than on CD4⁺ T cells and varies between donors and time points. Expression of OX40 was low on the depicted CD8⁺ T cells. FAP-targeted huCMP trimeric OX40 ligand-containing antigen binding molecules bound to OX40+ activated CD8⁺ T cells. Shown is the binding as median of fluorescence intensity (MFI) of FITC labeled anti-human IgG F(ab′)2 specific goat IgG F(ab′)2 fragment which is used as secondary detection antibody. MFI was measured by flow cytometry and baseline corrected by subtracting the MFI of the blank control. The x-axis shows the concentration of antigen binding molecules.

FIGS. 12A and 12B show the activation of NFκB by FAP-targeted huCMP trimeric OX40 ligand-containing antigen binding molecules in HeLa_hOx40_NFkB_Luc1 reporter cells in the absence (FIG. 12A) or presence of FAP positive tumor cells, respectively. In FIG. 12B is shown the activation of NF-κB signaling pathway in the reporter cells by FAP-targeted huCMP trimeric OX40 ligand-containing antigen binding molecules in the presence of low FAP expressing NIH-3T3 human FAP cells (ratio 3 FAP tumor cells to 1 reporter cell). The NF-κB-mediated luciferase activity was characterized by blotting the units of released light (URL), measured during 0.5 s, versus the concentration in nM of tested compounds. URLs are emitted due to luciferase-mediated oxidation of luciferin to oxyluciferin. Values are baseline corrected by subtracting the URLs of the blank control.

### DETAILED DESCRIPTION OF THE INVENTION

### Definitions

Unless defined otherwise, technical and scientific terms used herein have the same meaning as generally used in the art to which this invention belongs. For purposes of interpreting this specification, the following definitions will apply and whenever appropriate, terms used in the singular will also include the plural and vice versa.

As used herein, the term “antigen binding molecule” refers in its broadest sense to a molecule that specifically binds an antigenic determinant. Examples of antigen binding molecules are antibodies, antibody fragments and scaffold antigen binding proteins.

As used herein, the term “moiety capable of specific binding to a target cell antigen” refers to a polypeptide molecule that specifically binds to an antigenic determinant. In one aspect, the antigen binding moiety is able to activate signaling through its target cell antigen. In a particular aspect, the antigen binding moiety is able to direct the entity to which it is attached (e.g. the TNF family ligand trimer) to a target site, for example to a specific type of tumor cell or tumor stroma bearing the antigenic determinant. Moieties capable of specific binding to a target cell antigen include antibodies and fragments thereof as further defined herein. In addition, moieties capable of specific binding to a target cell antigen include scaffold antigen binding proteins as further defined herein, e.g. binding domains which are based on designed repeat proteins or designed repeat domains (see e.g. WO 2002/020565).

In relation to an antibody or fragment thereof, the term “moiety capable of specific binding to a target cell antigen” refers to the part of the molecule that comprises the area which specifically binds to and is complementary to part or all of an antigen. A moiety capable of specific antigen binding may be provided, for example, by one or more antibody variable domains (also called antibody variable regions). Particularly, a moiety capable of specific antigen binding comprises an antibody light chain variable region (VL) and an antibody heavy chain variable region (VH).

The term “antibody” herein is used in the broadest sense and encompasses various antibody structures, including but not limited to monoclonal antibodies, polyclonal antibodies, monospecific and multispecific antibodies (e.g., bispecific antibodies), and antibody fragments so long as they exhibit the desired antigen-binding activity.

The term “monoclonal antibody” as used herein refers to an antibody obtained from a population of substantially homogeneous antibodies, i.e., the individual antibodies comprising the population are identical and/or bind the same epitope, except for possible variant antibodies, e.g. containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen.

The term “monospecific” antibody as used herein denotes an antibody that has one or more binding sites each of which bind to the same epitope of the same antigen. The term “bispecific” means that the antigen binding molecule is able to specifically bind to at least two distinct antigenic determinants. Typically, a bispecific antigen binding molecule comprises two antigen binding sites, each of which is specific for a different antigenic determinant. In certain embodiments the bispecific antigen binding molecule is capable of simultaneously binding two antigenic determinants, particularly two antigenic determinants expressed on two distinct cells.

The term “valent” as used within the current application denotes the presence of a specified number of binding sites in an antigen binding molecule. As such, the terms “bivalent”, “tetravalent”, and “hexavalent” denote the presence of two binding sites, four binding sites, and six binding sites, respectively, in an antigen binding molecule.

The terms “full length antibody”, “intact antibody”, and “whole antibody” are used herein interchangeably to refer to an antibody having a structure substantially similar to a native antibody structure. “Native antibodies” refer to naturally occurring immunoglobulin molecules with varying structures. For example, native IgG-class antibodies are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a light chain constant domain (CL), also called a light chain constant region. The heavy chain of an antibody may be assigned to one of five types, called α (IgA), δ (IgD), ε (IgE), γ (IgG), or μ (IgM), some of which may be further divided into subtypes, e.g. γ1 (IgG1), γ2 (IgG2), γ3 (IgG3), γ4 (IgG4), α1 (IgA1) and α2 (IgA2). The light chain of an antibody may be assigned to one of two types, called kappa (κ) and lambda (λ), based on the amino acid sequence of its constant domain.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies, triabodies, tetrabodies, cross-Fab fragments; linear antibodies; single-chain antibody molecules (e.g. scFv); and single domain antibodies. For a review of certain antibody fragments, see Hudson et al., Nat Med 9, 129-134 (2003). For a review of scFv fragments, see e.g. Plückthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046. Diabodies are antibody fragments with two antigen-binding sites that may be bivalent or bispecific, see, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

Papain digestion of intact antibodies produces two identical antigen-binding fragments, called “Fab” fragments containing each the heavy- and light-chain variable domains and also the constant domain of the light chain and the first constant domain (CH1) of the heavy chain. As used herein, Thus, the term “Fab fragment” refers to an antibody fragment comprising a light chain fragment comprising a VL domain and a constant domain of a light chain (CL), and a VH domain and a first constant domain (CH1) of a heavy chain. Fab′ fragments differ from Fab fragments by the addition of a few residues at the carboxy terminus of the heavy chain CH1 domain including one or more cysteins from the antibody hinge region. Fab′-SH are Fab′ fragments in which the cysteine residue(s) of the constant domains bear a free thiol group. Pepsin treatment yields an F(ab′)₂ fragment that has two antigen-combining sites (two Fab fragments) and a part of the Fc region.

The term “cross-Fab fragment” or “xFab fragment” or “crossover Fab fragment” refers to a Fab fragment, wherein either the variable regions or the constant regions of the heavy and light chain are exchanged. Two different chain compositions of a crossover Fab molecule are possible and comprised in the bispecific antibodies of the invention: On the one hand, the variable regions of the Fab heavy and light chain are exchanged, i.e. the crossover Fab molecule comprises a peptide chain composed of the light chain variable region (VL) and the heavy chain constant region (CH1), and a peptide chain composed of the heavy chain variable region (VH) and the light chain constant region (CL). This crossover Fab molecule is also referred to as CrossFab _{(VLVH)}. On the other hand, when the constant regions of the Fab heavy and light chain are exchanged, the crossover Fab molecule comprises a peptide chain composed of the heavy chain variable region (VH) and the light chain constant region (CL), and a peptide chain composed of the light chain variable region (VL) and the heavy chain constant region (CH1). This crossover Fab molecule is also referred to as CrossFab _{(CLCL1)}.

A “single chain Fab fragment” or “scFab” is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL; and wherein said linker is a polypeptide of at least 30 amino acids, preferably between 32 and 50 amino acids. Said single chain Fab fragments are stabilized via the natural disulfide bond between the CL domain and the CH1 domain. In addition, these single chain Fab molecules might be further stabilized by generation of interchain disulfide bonds via insertion of cysteine residues (e.g. position 44 in the variable heavy chain and position 100 in the variable light chain according to Kabat numbering).

A “crossover single chain Fab fragment” or “x-scFab” is a is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CL-linker-VL-CH1 and b) VL-CH1-linker-VH-CL; wherein VH and VL form together an antigen-binding site which binds specifically to an antigen and wherein said linker is a polypeptide of at least 30 amino acids. In addition, these x-scFab molecules might be further stabilized by generation of interchain disulfide bonds via insertion of cysteine residues (e.g. position 44 in the variable heavy chain and position 100 in the variable light chain according to Kabat numbering).

A “single-chain variable fragment (scFv)” is a fusion protein of the variable regions of the heavy (V_{H}) and light chains (V_{L}) of an antibody, connected with a short linker peptide of ten to about 25 amino acids. The linker is usually rich in glycine for flexibility, as well as serine or threonine for solubility, and can either connect the N-terminus of the VH with the C-terminus of the V_{L}, or vice versa. This protein retains the specificity of the original antibody, despite removal of the constant regions and the introduction of the linker. scFv antibodies are, e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

“Scaffold antigen binding proteins” are known in the art, for example, fibronectin and designed ankyrin repeat proteins (DARPins) have been used as alternative scaffolds for antigen-binding domains, see, e.g., Gebauer and Skerra, Engineered protein scaffolds as next-generation antibody therapeutics. Curr Opin Chem Biol 13:245-255 (2009) and Stumpp et al., Darpins: A new generation of protein therapeutics. Drug Discovery Today 13: 695-701 (2008). In one aspect of the invention, a scaffold antigen binding protein is selected from the group consisting of CTLA-4 (Evibody), Lipocalins (Anticalin), a Protein A-derived molecule such as Z-domain of Protein A (Affibody), an A-domain (Avimer/Maxibody), a serum transferrin (trans-body); a designed ankyrin repeat protein (DARPin), a variable domain of antibody light chain or heavy chain (single-domain antibody, sdAb), a variable domain of antibody heavy chain (nanobody, aVH), V_{NAR} fragments, a fibronectin (AdNectin), a C-type lectin domain (Tetranectin); a variable domain of a new antigen receptor beta-lactamase (V_{NAR} fragments), a human gamma-crystallin or ubiquitin (Affilin molecules); a kunitz type domain of human protease inhibitors, microbodies such as the proteins from the knottin family, peptide aptamers and fibronectin (adnectin).

CTLA-4 (Cytotoxic T Lymphocyte-associated Antigen 4) is a CD28-family receptor expressed on mainly CD4+ T-cells. Its extracellular domain has a variable domain-like Ig fold. Loops corresponding to CDRs of antibodies can be substituted with heterologous sequence to confer different binding properties. CTLA-4 molecules engineered to have different binding specificities are also known as Evibodies (e.g. U.S. Pat. No. 7,166,697B1). Evibodies are around the same size as the isolated variable region of an antibody (e.g. a domain antibody). For further details see Journal of Immunological Methods 248 (1-2), 31-45 (2001).

Lipocalins are a family of extracellular proteins which transport small hydrophobic molecules such as steroids, bilins, retinoids and lipids. They have a rigid beta-sheet secondary structure with a number of loops at the open end of the conical structure which can be engineered to bind to different target antigens. Anticalins are between 160-180 amino acids in size, and are derived from lipocalins. For further details see Biochim Biophys Acta 1482: 337-350 (2000),

U.S. Pat. No. 7,250,297B1 and US20070224633.

An affibody is a scaffold derived from Protein A of Staphylococcus aureus which can be engineered to bind to antigen. The domain consists of a three-helical bundle of approximately 58 amino acids. Libraries have been generated by randomization of surface residues. For further details see Protein Eng. Des. Sel. 17, 455-462 (2004) and EP 1641818A1.

Avimers are multidomain proteins derived from the A-domain scaffold family. The native domains of approximately 35 amino acids adopt a defined disulfide bonded structure. Diversity is generated by shuffling of the natural variation exhibited by the family of A-domains. For further details see Nature Biotechnology 23(12), 1556-1561 (2005) and Expert Opinion on Investigational Drugs 16(6), 909-917 (June 2007).

A transferrin is a monomeric serum transport glycoprotein. Transferrins can be engineered to bind different target antigens by insertion of peptide sequences in a permissive surface loop. Examples of engineered transferrin scaffolds include the Trans-body. For further details see J. Biol. Chem 274, 24066-24073 (1999).

Designed Ankyrin Repeat Proteins (DARPins) are derived from Ankyrin which is a family of proteins that mediate attachment of integral membrane proteins to the cytoskeleton. A single ankyrin repeat is a 33 residue motif consisting of two alpha-helices and a beta-turn. They can be engineered to bind different target antigens by randomizing residues in the first alpha-helix and a beta-turn of each repeat. Their binding interface can be increased by increasing the number of modules (a method of affinity maturation). For further details see J. Mol. Biol. 332, 489-503 (2003), PNAS 100(4), 1700-1705 (2003) and J. Mol. Biol. 369, 1015-1028 (2007) and US20040132028A1.

A single-domain antibody is an antibody fragment consisting of a single monomeric variable antibody domain. The first single domains were derived from the variable domain of the antibody heavy chain from camelids (nanobodies or V_{H}H fragments). Furthermore, the term single-domain antibody includes an autonomous human heavy chain variable domain (aVH) or V_{NAR} fragments derived from sharks.

Fibronectin is a scaffold which can be engineered to bind to antigen. Adnectins consists of a backbone of the natural amino acid sequence of the 10th domain of the 15 repeating units of human fibronectin type III (FN3). Three loops at one end of the .beta.-sandwich can be engineered to enable an Adnectin to specifically recognize a therapeutic target of interest. For further details see Protein Eng. Des. Sel. 18, 435-444 (2005), US20080139791, WO2005056764 and U.S. Pat. No. 6,818,418B1.

Peptide aptamers are combinatorial recognition molecules that consist of a constant scaffold protein, typically thioredoxin (TrxA) which contains a constrained variable peptide loop inserted at the active site. For further details see Expert Opin. Biol. Ther. 5, 783-797 (2005).

Microbodies are derived from naturally occurring microproteins of 25-50 amino acids in length which contain 3-4 cysteine bridges—examples of microproteins include KalataBl and conotoxin and knottins. The microproteins have a loop which can beengineered to include upto 25 amino acids without affecting the overall fold of the microprotein. For further details of engineered knottin domains, see WO2008098796.

An “antigen binding molecule that binds to the same epitope” as a reference molecule refers to an antigen binding molecule that blocks binding of the reference molecule to its antigen in a competition assay by 50% or more, and conversely, the reference molecule blocks binding of the antigen binding molecule to its antigen in a competition assay by 50% or more.

The term “antigen binding domain” refers to the part of an antigen binding molecule that comprises the area which specifically binds to and is complementary to part or all of an antigen. Where an antigen is large, an antigen binding molecule may only bind to a particular part of the antigen, which part is termed an epitope. An antigen binding domain may be provided by, for example, one or more variable domains (also called variable regions). Preferably, an antigen binding domain comprises an antibody light chain variable region (VL) and an antibody heavy chain variable region (VH).

As used herein, the term “antigenic determinant” is synonymous with “antigen” and “epitope,” and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins useful as antigens herein can be any native form the proteins from any vertebrate source, including mammals such as primates (e.g. humans) and rodents (e.g. mice and rats), unless otherwise indicated. In a particular embodiment the antigen is a human protein. Where reference is made to a specific protein herein, the term encompasses the “full-length”, unprocessed protein as well as any form of the protein that results from processing in the cell. The term also encompasses naturally occurring variants of the protein, e.g. splice variants or allelic variants.

By “specific binding” is meant that the binding is selective for the antigen and can be discriminated from unwanted or non-specific interactions. The ability of an antigen binding molecule to bind to a specific antigen can be measured either through an enzyme-linked immunosorbent assay (ELISA) or other techniques familiar to one of skill in the art, e.g. Surface Plasmon Resonance (SPR) technique (analyzed on a BIAcore instrument) (Liljeblad et al., Glyco J 17, 323-329 (2000)), and traditional binding assays (Heeley, Endocr Res 28, 217-229 (2002)). In one embodiment, the extent of binding of an antigen binding molecule to an unrelated protein is less than about 10% of the binding of the antigen binding molecule to the antigen as measured, e.g. by SPR. In certain embodiments, an molecule that binds to the antigen has a dissociation constant (Kd) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g. from 10⁻⁹ M to 10⁻¹³ M).

“Affinity” or “binding affinity” refers to the strength of the sum total of non-covalent interactions between a single binding site of a molecule (e.g. an antibody) and its binding partner (e.g. an antigen). Unless indicated otherwise, as used herein, “binding affinity” refers to intrinsic binding affinity which reflects a 1:1 interaction between members of a binding pair (e.g. antibody and antigen). The affinity of a molecule X for its partner Y can generally be represented by the dissociation constant (Kd), which is the ratio of dissociation and association rate constants (koff and kon, respectively). Thus, equivalent affinities may comprise different rate constants, as long as the ratio of the rate constants remains the same. Affinity can be measured by common methods known in the art, including those described herein. A particular method for measuring affinity is Surface Plasmon Resonance (SPR).

A “target cell antigen” as used herein refers to an antigenic determinant presented on the surface of a target cell, for example a cell in a tumor such as a cancer cell or a cell of the tumor stroma. In certain embodiments, the target cell antigen is an antigen on the surface of a tumor cell. In one embodiment, target cell antigen is selected from the group consisting of Fibroblast Activation Protein (FAP), Carcinoembryonic Antigen (CEA), Melanoma-associated Chondroitin Sulfate Proteoglycan (MCSP), Epidermal Growth Factor Receptor (EGFR), CD19, CD20 and CD33. In particular, the target cell antigen is Fibroblast Activation Protein (FAP).

The term “Fibroblast activation protein (FAP)”, also known as Prolyl endopeptidase FAP or Seprase (EC 3.4.21), refers to any native FAP from any vertebrate source, including mammals such as primates (e.g. humans) non-human primates (e.g. cynomolgus monkeys) and rodents (e.g. mice and rats), unless otherwise indicated. The term encompasses “full-length,” unprocessed FAP as well as any form of FAP that results from processing in the cell. The term also encompasses naturally occurring variants of FAP, e.g., splice variants or allelic variants. In one embodiment, the antigen binding molecule of the invention is capable of specific binding to human, mouse and/or cynomolgus FAP. The amino acid sequence of human FAP is shown in UniProt (www.uniprot.org) accession no. Q12884 (version 149, SEQ ID NO:30), or NCBI (www.ncbi.nlm.nih.gov/) RefSeq NP_004451.2. The extracellular domain (ECD) of human FAP extends from amino acid position 26 to 760. The amino acid and nucleotide sequences of a His-tagged human FAP ECD is shown in SEQ ID NOs 31 and 32, respectively. The amino acid sequence of mouse FAP is shown in UniProt accession no. P97321 (version 126, SEQ ID NO:33), or NCBI RefSeq NP_032012.1. The extracellular domain (ECD) of mouse FAP extends from amino acid position 26 to 761. SEQ ID NOs 34 and 35 show the amino acid and nucleotide sequences, respectively, of a His-tagged mouse FAP ECD. SEQ ID NOs 36 and 37 show the amino acid and nucleotide sequences, respectively, of a His-tagged cynomolgus FAP ECD. Preferably, an anti-FAP binding molecule of the invention binds to the extracellular domain of FAP. Exemplary anti-FAP binding molecules are described in International Patent Application No. WO 2012/020006 A2.

The term “Carcinoembroynic antigen (CEA)”, also known as Carcinoembryonic antigen-related cell adhesion molecule 5 (CEACAM5), refers to any native CEA from any vertebrate source, including mammals such as primates (e.g. humans) non-human primates (e.g. cynomolgus monkeys) and rodents (e.g. mice and rats), unless otherwise indicated. The amino acid sequence of human CEA is shown in UniProt accession no. P06731 (version 151, SEQ ID NO:38). CEA has long been identified as a tumor-associated antigen (Gold and Freedman, J Exp Med., 121:439-462, 1965; Berinstein N. L., J Clin Oncol., 20:2197-2207, 2002). Originally classified as a protein expressed only in fetal tissue, CEA has now been identified in several normal adult tissues. These tissues are primarily epithelial in origin, including cells of the gastrointestinal, respiratory, and urogential tracts, and cells of colon, cervix, sweat glands, and prostate (Nap et al., Tumour Biol., 9(2-3):145-53, 1988; Nap et al., Cancer Res., 52(8):2329-23339, 1992). Tumors of epithelial origin, as well as their metastases, contain CEA as a tumor associated antigen. While the presence of CEA itself does not indicate transformation to a cancerous cell, the distribution of CEA is indicative. In normal tissue, CEA is generally expressed on the apical surface of the cell (Hammarstrom S., Semin Cancer Biol. 9(2):67-81 (1999)), making it inaccessible to antibody in the blood stream. In contrast to normal tissue, CEA tends to be expressed over the entire surface of cancerous cells (Hammarstrom S., Semin Cancer Biol. 9(2):67-81 (1999)). This change of expression pattern makes CEA accessible to antibody binding in cancerous cells. In addition, CEA expression increases in cancerous cells. Furthermore, increased CEA expression promotes increased intercellular adhesions, which may lead to metastasis (Marshall J., Semin Oncol., 30(a Suppl. 8):30-6, 2003). The prevalence of CEA expression in various tumor entities is generally very high. In concordance with published data, own analyses performed in tissue samples confirmed its high prevalence, with approximately 95% in colorectal carcinoma (CRC), 90% in pancreatic cancer, 80% in gastric cancer, 60% in non-small cell lung cancer (NSCLC, where it is co-expressed with HERS), and 40% in breast cancer; low expression was found in small cell lung cancer and glioblastoma.

CEA is readily cleaved from the cell surface and shed into the blood stream from tumors, either directly or via the lymphatics. Because of this property, the level of serum CEA has been used as a clinical marker for diagnosis of cancers and screening for recurrence of cancers, particularly colorectal cancer (Goldenberg D M., The International Journal of Biological Markers, 7:183-188, 1992; Chau I., et al., J Clin Oncol., 22:1420-1429, 2004; Flamini et al., Clin Cancer Res; 12(23):6985-6988, 2006).

The term “Melanoma-associated Chondroitin Sulfate Proteoglycan (MCSP)”, also known as Chondroitin Sulfate Proteoglycan 4 (CSPG4) refers to any native MCSP from any vertebrate source, including mammals such as primates (e.g. humans) non-human primates (e.g. cynomolgus monkeys) and rodents (e.g. mice and rats), unless otherwise indicated. The amino acid sequence of human MCSP is shown in UniProt accession no. Q6UVK1 (version 103, SEQ ID NO:39). The term “Epidermal Growth Factor Receptor (EGFR)”, also named Proto-oncogene c-ErbB-1 or Receptor tyrosine-protein kinase erbB-1, refers to any native EGFR from any vertebrate source, including mammals such as primates (e.g. humans) non-human primates (e.g. cynomolgus monkeys) and rodents (e.g. mice and rats), unless otherwise indicated. The amino acid sequence of human EGFR is shown in UniProt accession no. P00533 (version 211, SEQ ID NO:40).

The term “CD19” refers to B-lymphocyte antigen CD19, also known as B-lymphocyte surface antigen B4 or T-cell surface antigen Leu-12 and includes any native CD19 from any vertebrate source, including mammals such as primates (e.g. humans) non-human primates (e.g. cynomolgus monkeys) and rodents (e.g. mice and rats), unless otherwise indicated. The amino acid sequence of human CD19 is shown in Uniprot accession no. P15391 (version 160, SEQ ID NO:41). The term encompasses “full-length” unprocessed human CD19 as well as any form of human CD19 that results from processing in the cell as long as the antibody as reported herein binds thereto. CD19 is a structurally distinct cell surface receptor expressed on the surface of human B cells, including, but not limited to, pre-B cells, B cells in early development {i.e., immature B cells), mature B cells through terminal differentiation into plasma cells, and malignant B cells. CD19 is expressed by most pre-B acute lymphoblastic leukemias (ALL), non-Hodgkin's lymphomas, B cell chronic lymphocytic leukemias (CLL), pro-lymphocytic leukemias, hairy cell leukemias, common acute lymphocytic leukemias, and some Null-acute lymphoblastic leukemias. The expression of CD19 on plasma cells further suggests it may be expressed on differentiated B cell tumors such as multiple myeloma. Therefore, the CD19 antigen is a target for immunotherapy in the treatment of non-Hodgkin's lymphoma, chronic lymphocytic leukemia and/or acute lymphoblastic leukemia.

“CD20” refers to B-lymphocyte antigen CD20, also known as membrane-spanning 4-domains subfamily A member 1 (MS4A1), B-lymphocyte surface antigen B1 or Leukocyte surface antigen Leu-16, and includes any native CD20 from any vertebrate source, including mammals such as primates (e.g. humans) non-human primates (e.g. cynomolgus monkeys) and rodents (e.g. mice and rats), unless otherwise indicated. The amino acid sequence of human CD20 is shown in Uniprot accession no. P11836 (version 149, SEQ ID NO:42). “CD33” refers to Myeloid cell surface antigen CD33, also known as SIGLEC3 or gp67, and includes any native CD33 from any vertebrate source, including mammals such as primates (e.g. humans) non-human primates (e.g. cynomolgus monkeys) and rodents (e.g. mice and rats), unless otherwise indicated. The amino acid sequence of human CD33 is shown in Uniprot accession no. P20138 (version 157, SEQ ID NO:43).

The term “variable region” or “variable domain” refers to the domain of an antibody heavy or light chain that is involved in binding the antigen binding molecule to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6th ed., W.H. Freeman and Co., page 91 (2007). A single VH or VL domain may be sufficient to confer antigen-binding specificity.

The term “hypervariable region” or “HVR,” as used herein refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987).) Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, Md. (1991).) Hypervariable regions (HVRs) are also referred to as complementarity determining regions (CDRs), and these terms are used herein interchangeably in reference to portions of the variable region that form the antigen binding regions. This particular region has been described by Kabat et al., U.S. Dept. of Health and Human Services, “Sequences of Proteins of Immunological Interest” (1983) and by Chothia et al., J. Mol. Biol. 196:901-917 (1987), where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth below in Table A as a comparison. The exact residue numbers which encompass a particular CDR will vary depending on the sequence and size of the CDR. Those skilled in the art can routinely determine which residues comprise a particular CDR given the variable region amino acid sequence of the antibody

TABLE A
CDR Definitions¹

| CDR | Kabat | Chothia | AbM² |
|---|---|---|---|
| V_{H} CDR1 | 31-35 | 26-32 | 26-35 |
| V_{H} CDR2 | 50-65 | 52-58 | 50-58 |
| V_{H} CDR3 | 95-102 | 95-102 | 95-102 |
| V_{L} CDR1 | 24-34 | 26-32 | 24-34 |
| V_{L} CDR2 | 50-56 | 50-52 | 50-56 |
| V_{L} CDR3 | 89-97 | 91-96 | 89-97 |

¹Numbering of all CDR definitions in Table A is according to the numbering conventions set forth by Kabat et al. (see below).
²“AbM” with a lowercase “b” as used in Table A refers to the CDRs as defined by Oxford Molecular's “AbM” antibody modeling software.

Kabat et al. also defined a numbering system for variable region sequences that is applicable to any antibody. One of ordinary skill in the art can unambiguously assign this system of “Kabat numbering” to any variable region sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless otherwise specified, references to the numbering of specific amino acid residue positions in an antibody variable region are according to the Kabat numbering system.

With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See Almagro and Fransson, Front. Biosci. 13:1619-1633 (2008).) Unless otherwise indicated, HVR residues and other residues in the variable domain (e.g., FR residues) are numbered herein according to Kabat et al., supra.

As used herein, the term “affinity matured” in the context of antigen binding molecules (e.g., antibodies) refers to an antigen binding molecule that is derived from a reference antigen binding molecule, e.g., by mutation, binds to the same antigen, preferably binds to the same epitope, as the reference antibody; and has a higher affinity for the antigen than that of the reference antigen binding molecule. Affinity maturation generally involves modification of one or more amino acid residues in one or more CDRs of the antigen binding molecule. Typically, the affinity matured antigen binding molecule binds to the same epitope as the initial reference antigen binding molecule.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g. IgG₁, IgG₂, IgG₃, IgG₄, IgA₁, and IgA₂. The heavy chain constant domains that correspond to the different classes of immunoglobulins are called α, δ, ε, γ, and μ respectively.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. Other forms of “humanized antibodies” encompassed by the present invention are those in which the constant region has been additionally modified or changed from that of the original antibody to generate the properties according to the invention, especially in regard to C1q binding and/or Fc receptor (FcR) binding.

A “human” antibody is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “Fc domain” or “Fc region” herein is used to define a C-terminal region of an antibody heavy chain that contains at least a portion of the constant region. The term includes native sequence Fc regions and variant Fc regions. An IgG Fc region comprises an IgG CH2 and an IgG CH3 domain. The “CH2 domain” of a human IgG Fc region usually extends from an amino acid residue at about position 231 to an amino acid residue at about position 340. In one embodiment, a carbohydrate chain is attached to the CH2 domain. The CH2 domain herein may be a native sequence CH2 domain or variant CH2 domain. The “CH3 domain” comprises the stretch of residues C-terminal to a CH2 domain in an Fc region (i.e. from an amino acid residue at about position 341 to an amino acid residue at about position 447 of an IgG). The CH3 region herein may be a native sequence CH3 domain or a variant CH3 domain (e.g. a CH3 domain with an introduced “protuberance” (“knob”) in one chain thereof and a corresponding introduced “cavity” (“hole”) in the other chain thereof; see U.S. Pat. No. 5,821,333, expressly incorporated herein by reference). Such variant CH3 domains may be used to promote heterodimerization of two non-identical antibody heavy chains as herein described. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

The term “TNF ligand family member” or “TNF family ligand” refers to a proinflammatory cytokine. Cytokines in general, and in particular the members of the TNF ligand family, play a crucial role in the stimulation and coordination of the immune system. At present, nineteen cyctokines have been identified as members of the TNF (tumour necrosis factor) ligand superfamily on the basis of sequence, functional, and structural similarities. All these ligands are type II transmembrane proteins with a C-terminal extracellular domain (ectodomain), N-terminal intracellular domain and a single transmembrane domain. The C-terminal extracellular domain, known as TNF homology domain (THD), has 20-30% amino acid identity between the superfamily members and is responsible for binding to the receptor. The TNF ectodomain is also responsible for the TNF ligands to form trimeric complexes that are recognized by their specific receptors. Members of the TNF ligand family are selected from the group consisting of Lymphotoxin α (also known as LTA or TNFSF1), TNF (also known as TNFSF2), LTβ (also known as TNFSF3), OX40L (also known as TNFSF4), CD40L (also known as CD154 or TNFSF5), FasL (also known as CD95L, CD178 or TNFSF6), CD27L (also known as CD70 or TNFSF7), CD30L (also known as CD153 or TNFSF8), 4-1BBL (also known as TNFSF9), TRAIL (also known as APO2L, CD253 or TNFSF10), RANKL (also known as CD254 or TNFSF11), TWEAK (also known as TNFSF12), APRIL (also known as CD256 or TNFSF13), BAFF (also known as CD257 or TNFSF13B), LIGHT (also known as CD258 or TNFSF14), TL1A (also known as VEGI or TNFSF15), GITRL (also known as TNFSF18), EDA-A1 (also known as ectodysplasin A1) and EDA-A2 (also known as ectodysplasin A2). The term refers to any native TNF family ligand from any vertebrate source, including mammals such as primates (e.g. humans), non-human primates (e.g. cynomolgus monkeys) and rodents (e.g. mice and rats), unless otherwise indicated.

The term “costimulatory TNF ligand family member” or “costimulatory TNF family ligand” refers to a subgroup of TNF ligand family members, which are able to costimulate proliferation and cytokine production of T-cells. These TNF family ligands can costimulate TCR signals upon interaction with their corresponding TNF receptors and the interaction with their receptors leads to recruitment of TNFR-associated factors (TRAF), which initiate signalling cascades that result in T-cell activation. Costimulatory TNF family ligands are selected from the group consisting of 4-1BBL, OX40L, GITRL, CD70, CD30L and LIGHT, more particularly the costimulatory TNF ligand family member is selected from 4-1BBL, OX40L and GITRL, most particularly from 4-1BBL and OX40L.

Further information, in particular sequences, of the costimulatory TNF ligand family members may be obtained from publically accessible databases such as Uniprot (www.uniprot.org). For instance, the human TNF ligands have the following amino acid sequences: human OX40L (UniProt accession no. P23510, SEQ ID NO:44), human CD27L (CD70, UniProt accession no. P32970, SEQ ID NO:45), human CD30L (UniProt accession no. P32971, SEQ ID NO:46), 4-1BBL (UniProt accession no. P41273, SEQ ID NO:47), LIGHT (UniProt accession no. 043557, SEQ ID NO:48), and GITRL (UniProt accession no. Q9UNG2, SEQ ID NO:49).

An “ectodomain” is the domain of a membrane protein that extends into the extracellular space (i.e. the space outside the target cell). Ectodomains are usually the parts of proteins that initiate contact with surfaces, which leads to signal transduction. The ectodomain of TNF ligand family member as defined herein thus refers to the part of the TNF ligand protein that extends into the extracellular space (the extracellular domain), but also includes shorter parts or fragments thereof that are responsible for the trimerization and for the binding to the corresponding TNF receptor. The term “ectodomain of a TNF ligand family member or a fragment thereof” thus refers to the extracellular domain of the TNF ligand family member that forms the extracellular domain or to parts thereof that are still able to bind to the receptor (receptor binding domain).

As described herein before, 4-1BBL is a type II transmembrane protein and one member of the TNF ligand family. Complete or full length 4-1BBL having the amino acid sequence of SEQ ID NO:47 has been described to form trimers on the surface of cells. The formation of trimers is enabled by specific motives of the ectodomain of 4-1BBL. Said motives are designated herein as “trimerization region”. The amino acids 50-254 of the human 4-1BBL sequence (SEQ ID NO:50) form the extracellular domain of 4-1BBL, but even fragments thereof are able to form the trimers. In specific embodiments of the invention, the term “ectodomain of 4-1BBL or a fragment thereof” refers to a polypeptide having an amino acid sequence selected from SEQ ID NO:6 (amino acids 52-254 of human 4-1BBL), SEQ ID NO:3 (amino acids 71-254 of human 4-1BBL), SEQ ID NO:5 (amino acids 80-254 of human 4-1BBL) and SEQ ID NO:4 (amino acids 85-254 of human 4-1BBL) or a polypeptide having an amino acid sequence selected from SEQ ID NO:7 (amino acids 71-248 of human 4-1BBL), SEQ ID NO:10 (amino acids 52-248 of human 4-1BBL), SEQ ID NO:9 (amino acids 80-248 of human 4-1BBL) and SEQ ID NO:8 (amino acids 85-248 of human 4-1BBL), but also other fragments of the ectodomain capable of trimerization are included herein.

As described herein before, OX40L is another type II transmembrane protein and a further member of the TNF ligand family. Complete or full length human OX40L has the amino acid sequence of SEQ ID NO:44. The amino acids 51-183 of the human OX40L sequence (SEQ ID NO:11) form the extracellular domain of OX40L, but even fragments thereof that are able to form the trimers. In specific embodiments of the invention, the term “ectodomain of OX40L or a fragment thereof” refers to a polypeptide having an amino acid sequence selected from SEQ ID NO:11 (amino acids 51-183 of human OX40L) or SEQ ID NO:13 (amino acids 52-183 of human OX40L), but also other fragments of the ectodomain capable of trimerization are included herein.

The term “peptide linker” refers to a peptide comprising one or more amino acids, typically about 2 to 20 amino acids. Peptide linkers are known in the art or are described herein. Suitable, non-immunogenic linker peptides are, for example, (G₄S)n, (SG₄)ₙ or G₄(SG₄)ₙ peptide linkers, wherein “n” is generally a number between 1 and 10, typically between 1 and 4, in particular 2, i.e. the peptides selected from the group consisting of GGGGS (SEQ ID NO:51), GGGGSGGGGS (SEQ ID NO:52), SGGGGSGGGG (SEQ ID NO:53), (G₄S)₃ or GGGGSGGGGSGGGGS (SEQ ID NO:54), GGGGSGGGGSGGGG or G4(SG4)₂ (SEQ ID NO:55), and (G₄S)₄ or GGGGSGGGGSGGGGSGGGGS (SEQ ID NO:56), but also include the sequences GSPGSSSSGS (SEQ ID NO:57), GSGSGSGS (SEQ ID NO:58), GSGSGNGS (SEQ ID NO:59), GGSGSGSG (SEQ ID NO:60), GGSGSG (SEQ ID NO:61), GGSG (SEQ ID NO:62), GGSGNGSG (SEQ ID NO:63), GGNGSGSG (SEQ ID NO:64) and GGNGSG (SEQ ID NO:65). Peptide linkers of particular interest are (G4S)₁ or GGGGS (SEQ ID NO:51), (G₄S)₂ or GGGGSGGGGS (SEQ ID NO:52), (G₄S)₃ or GGGGSGGGGSGGGGS (SEQ ID NO:54) and (G₄S)₄ or GGGGSGGGGSGGGGSGGGGS (SEQ ID NO:56).

The term “amino acid” as used within this application denotes the group of naturally occurring carboxy α-amino acids comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

A “fusion polypeptide” as used herein refers to a single chain polypeptide composed of an ectodomain of a TNF ligand family member fused to a trimerization domain. In addition, the fusion polypeptide may further comprise a moiety capable of specific binding to a target cell antigen, in particular a Fab fragment, more particularly the CH1 and VH domain of a Fab fragment. The fusion may occur by directly linking the N or C-terminal amino acid of the antigen binding moiety via a peptide linker to the C- or N-terminal amino acid of the ectodomain of said TNF ligand family member.

By “fused” or “connected” is meant that the components (e.g. a polypeptide and an ectodomain of said TNF ligand family member) are linked by peptide bonds, either directly or via one or more peptide linkers.

The term “trimerization domain” refers to an amino acid sequence within a polypeptide that promotes self-assembly by associating with two other trimerization domains to form a trimer. The term is also use to refer to the polynucleotide encoding said amino acid sequence. Typically, the trimerization domain comprises an amino acid sequence able to form an alpha-helicial coiled-coil domain or an isoleucine zipper domain. Suitable trimerization domains include TRAF2 (UniProt accession no. Q12933, SEQ ID NO:66), in particular amino acids 299 to 348 or amino acids 310 to 349), Thrombospondin 1 (UniProt accession no. P07996, SEQ ID NO:67), in particular amino acids 291 to 314), Matrilin-4 (UniProt accession no. 095460, SEQ ID NO:68), in particular amino acids 594 to 618; CMP (matrilin-1) (Uniprot accession No. P21941, SEQ ID NO:1), in particular amino acids 454 to 496, and Cubilin (UniProt accession no. 060494, SEQ ID NO: 69), in particular amino acids 104 to 138. An exemplary isoleucine zipper domain is the engineered yeast GCN4 isoleucine variant described by Harbury et al. (1993) Science 262, 1401-1407 comprising the amino acid sequence of SEQ ID NO:70.

A particular trimerization domain is “huCMP” or “CMP” or “human cartilage matrix protein”, a protein that is also known as matrilin-1 or MATN1 or CRTM (UniProt accession no. P21941, SEQ ID NO:1). The term “huCMP trimerization domain” or “trimerization domain derived from human cartilage matrix protein (CMP)” refers to a polypeptide structure capable of associating with two similar or identical polypeptides to form a stable trimer. The trimerisation is mediated through ionic bonds and other non-covalent bonds formed between adjacent charged amino acids of the polypeptide chains. The huCMP trimerization domain has been been described e.g. in Beck et al (1996), J. Mol. Biol. 256, 909-923. A huCMP trimerization domain of particular interes comprises a sequence having at least 95% identity and most preferably at least 98% identity to SEQ ID NO 2. In one embodiment said trimerization domain comprises the sequence of SEQ ID NO. 2.

“Percent (%) amino acid sequence identity” with respect to a reference polypeptide (protein) sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN. SAWI or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In certain embodiments, amino acid sequence variants of the TNF ligand trimer-containing antigen binding molecules provided herein are contemplated. For example, it may be desirable to improve the binding affinity and/or other biological properties of the TNF ligand trimer-containing antigen binding molecules. Amino acid sequence variants of the TNF ligand trimer-containing antigen binding molecules may be prepared by introducing appropriate modifications into the nucleotide sequence encoding the molecules, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding. Sites of interest for substitutional mutagenesis include the HVRs and Framework (FRs). Conservative substitutions are provided in Table B under the heading “Preferred Substitutions” and further described below in reference to amino acid side chain classes (1) to (6). Amino acid substitutions may be introduced into the molecule of interest and the products screened for a desired activity, e.g., retained/improved antigen binding, decreased immunogenicity, or improved ADCC or CDC.

TABLE B

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

The term “amino acid sequence variants” includes substantial variants wherein there are amino acid substitutions in one or more hypervariable region residues of a parent antigen binding molecule (e.g. a humanized or human antibody). Generally, the resulting variant(s) selected for further study will have modifications (e.g., improvements) in certain biological properties (e.g., increased affinity, reduced immunogenicity) relative to the parent antigen binding molecule and/or will have substantially retained certain biological properties of the parent antigen binding molecule. An exemplary substitutional variant is an affinity matured antibody, which may be conveniently generated, e.g., using phage display-based affinity maturation techniques such as those described herein. Briefly, one or more HVR residues are mutated and the variant antigen binding molecules displayed on phage and screened for a particular biological activity (e.g. binding affinity). In certain embodiments, substitutions, insertions, or deletions may occur within one or more HVRs so long as such alterations do not substantially reduce the ability of the antigen binding molecule to bind antigen. For example, conservative alterations (e.g., conservative substitutions as provided herein) that do not substantially reduce binding affinity may be made in HVRs. A useful method for identification of residues or regions of an antibody that may be targeted for mutagenesis is called “alanine scanning mutagenesis” as described by Cunningham and Wells (1989) Science, 244:1081-1085.

In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antigen binding molecule complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include the fusion to the N- or C-terminus to a polypeptide which increases the serum half-life of the trimeric costimulatory TNF family ligand-containing antigen binding molecules.

In certain aspects, the trimeric costimulatory TNF family ligand-containing antigen binding molecules provided herein are altered to increase or decrease the extent to which the antibody is glycosylated. Glycosylation variants of the molecules may be conveniently obtained by altering the amino acid sequence such that one or more glycosylation sites is created or removed. In certain aspects, the trimeric costimulatory TNF family ligand-containing antigen binding molecules provided herein may be further modified to contain additional non-proteinaceous moieties that are known in the art and readily available. The moieties suitable for derivatization of the antigen binding molecule include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antigen binding molecule to be improved.

The term “polynucleotide” refers to an isolated nucleic acid molecule or construct, e.g. messenger RNA (mRNA), virally-derived RNA, or plasmid DNA (pDNA). A polynucleotide may comprise a conventional phosphodiester bond or a non-conventional bond (e.g. an amide bond, such as found in peptide nucleic acids (PNA). The term “nucleic acid molecule” refers to any one or more nucleic acid segments, e.g. DNA or RNA fragments, present in a polynucleotide.

By “isolated” nucleic acid molecule or polynucleotide is intended a nucleic acid molecule, DNA or RNA, which has been removed from its native environment. For example, a recombinant polynucleotide encoding a polypeptide contained in a vector is considered isolated for the purposes of the present invention. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extrachromosomally or at a chromosomal location that is different from its natural chromosomal location. Isolated RNA molecules include in vivo or in vitro RNA transcripts of the present invention, as well as positive and negative strand forms, and double-stranded forms. Isolated polynucleotides or nucleic acids according to the present invention further include such molecules produced synthetically. In addition, a polynucleotide or a nucleic acid may be or may include a regulatory element such as a promoter, ribosome binding site, or a transcription terminator.

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs, such as the ones discussed above for polypeptides (e.g. ALIGN-2).

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In certain embodiments, the expression cassette of the invention comprises polynucleotide sequences that encode bispecific antigen binding molecules of the invention or fragments thereof.

The term “vector” or “expression vector” is synonymous with “expression construct” and refers to a DNA molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery. In one embodiment, the expression vector of the invention comprises an expression cassette that comprises polynucleotide sequences that encode bispecific antigen binding molecules of the invention or fragments thereof.

The terms “host cell”, “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages.

Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the bispecific antigen binding molecules of the present invention. Host cells include cultured cells, e.g. mammalian cultured cells, such as CHO cells, BHK cells, NS0 cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

An “effective amount” of an agent refers to the amount that is necessary to result in a physiological change in the cell or tissue to which it is administered.

A “therapeutically effective amount” of an agent, e.g. a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

An “individual” or “subject” is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). Particularly, the individual or subject is a human.

The term “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable excipient” refers to an ingredient in a pharmaceutical composition, other than an active ingredient, which is nontoxic to a subject. A pharmaceutically acceptable excipient includes, but is not limited to, a buffer, a stabilizer, or a preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, the molecules of the invention are used to delay development of a disease or to slow the progression of a disease.

The term “cancer” as used herein refers to proliferative diseases, such as lymphomas, lymphocytic leukemias, lung cancer, non-small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma and Ewings sarcoma, including refractory versions of any of the above cancers, or a combination of one or more of the above cancers.

### Trimeric Costimulatory TNF Family Ligand-Containing Antigen Binding Molecules of the Invention

The invention provides novel trimeric costimulatory TNF family ligand-containing antigen binding molecules with particularly advantageous properties such as producibility, stability, binding affinity, biological activity, targeting efficiency and reduced toxicity.

The invention provides a trimeric costimulatory TNF family ligand-containing antigen binding molecule comprising three fusion polypeptides, each of the three fusion polypeptides comprising

(a) an ectodomain of a costimulatory TNF family ligand selected from the group consisting of 4-1BBL, OX40L and GITRL or fragments thereof,
(b) a trimerization domain derived from human cartilage matrix protein (huCMP) of amino acid sequence of SEQ ID NO:1, and
(c) a moiety capable of specific binding to a target cell antigen.

Thus, disclosed herein is a trimeric costimulatory TNF family ligand-containing antigen binding molecule comprising three fusion polypeptides, each of the three fusion polypeptides comprising

(a) an ectodomain of a co stimulatory TNF ligand family member or fragments thereof,
(b) a trimerization domain, and
(c) a moiety capable of specific binding to a target cell antigen.

A trimerization domain can be derived from a polypeptide derived from a protein selected from the group consisting of human TRAF2, human thrombospondin 1, human matrilin-4, human cartilage matrix protein (huCMP) and human cubilin. It can also be an isoleucine zipper domain, for example the peptide with the amino acid sequence of SEQ ID NO:70. A trimeric costimulatory TNF family ligand-containing antigen binding molecule of the invention comprises a trimerization domain derived from human cartilage matrix protein (huCMP) of amino acid sequence of SEQ ID NO:1.

The trimerization domain derived from human cartilage matrix protein (huCMP) comprises at least a part of SEQ ID NO.: 1. In one aspect, the trimerization domain comprises an amino acid sequence having at least 95% identity and most preferably at least 98% identity to SEQ ID NO:2. More particularly, the trimerization domain comprises the amino acid sequence of SEQ ID NO:2. The trimerization domain derived from human cartilage matrix protein (CMP) is herein further referred to as “huCMP trimerization domain”.

In a particular aspect, the trimeric costimulatory TNF family ligand-containing antigen binding molecule comprises three fusion polypeptides, each of the three fusion polypeptides comprising a trimerization domain comprising an amino acid sequence having at least 95% identity and in particular at least 98% identity to SEQ ID NO:2. More particularly, each of the three fusion polypeptides comprises one trimerization domain comprising the amino acid sequence of SEQ ID NO:2.

In particular, the costimulatory TNF family ligand member is selected from the group consisting of 4-1BBL, OX40L and GITRL. More particularly the costimulatory TNF family ligand member is selected from 4-1BBL and OX40L.

In one aspect, the costimulatory TNF ligand family member is 4-1BBL. In particular, the trimeric costimulatory TNF family ligand-containing antigen binding molecule comprises three fusion polypeptides, wherein in each of the three fusion polypeptides the ectodomain of a TNF family ligand comprises the amino acid sequence selected from the group consisting of SEQ ID NO:3, SEQ ID NO:4, SEQ ID NO:5, SEQ ID NO:6, SEQ ID NO:7, SEQ ID NO:8, SEQ ID NO:9 and SEQ ID NO:10, particularly the amino acid sequence of SEQ ID NO:3 or SEQ ID NO:7. In a further aspect, the trimeric costimulatory TNF family ligand-containing antigen binding molecule comprises three fusion polypeptides, each of the three fusion polypeptides comprising (a) an ectodomain of a TNF family ligand comprising the amino acid sequence selected from the group consisting of SEQ ID NO:3, SEQ ID NO:4, SEQ ID NO:5, SEQ ID NO:6, SEQ ID NO:7, SEQ ID NO:8, SEQ ID NO:9 and SEQ ID NO:10, (b) a trimerization domain comprising the amino acid sequence of SEQ ID NO:2 and (c) a moiety capable of specific binding to a target cell antigen.

In another aspect, the costimulatory TNF ligand family member is OX40L. In particular, the trimeric costimulatory TNF family ligand-containing antigen binding molecule comprises three fusion polypeptides, wherein in each of the three fusion polypeptides the ectodomain of a TNF family ligand comprises the amino acid sequence selected from the group consisting of SEQ ID NO:11, SEQ ID NO:12 and SEQ ID NO:13. In a further aspect, the trimeric costimulatory TNF family ligand-containing antigen binding molecule comprises three fusion polypeptides, wherein each of the three fusion polypeptides comprises (a) an ectodomain of a TNF family ligand comprising the amino acid sequence selected from the group consisting of SEQ ID NO:11, SEQ ID NO:12 and SEQ ID NO:13, (b) a trimerization domain comprising the amino acid sequence of SEQ ID NO:2 and (c) a moiety capable of specific binding to a target cell antigen.

In another aspect, provided is a trimeric costimulatory TNF family ligand-containing antigen binding molecule comprising three fusion polypeptides as described before, wherein the ectodomain of the TNF family ligand or a fragment thereof is fused at the N-terminal amino acid to the C-terminal amino acid of the trimerization domain, optionally through a peptide linker. The fusion can be a direct bond between the ectodomain of the TNF family ligand and the trimerization domain, or the ectodomain of the TNF family ligand and the trimerization domain are connected through a peptide linker. In a particular aspect, the ectodomain of the TNF family ligand and the trimerization domain are connected through a peptide linker.

Typically, the peptide linker is a peptide comprising 2 to 20 amino acids. In particular, the peptide linker is a peptide selected from the group consisting of GGGGS (SEQ ID NO:51), GGGGSGGGGS (SEQ ID NO:52), SGGGGSGGGG (SEQ ID NO:53), (G₄S)₃ or GGGGSGGGGSGGGGS (SEQ ID NO:54), GGGGSGGGGSGGGG or G4(SG4)2 (SEQ ID NO:55), and (G₄S)₄ or GGGGSGGGGSGGGGSGGGGS (SEQ ID NO:56), but also include the sequences GSPGSSSSGS (SEQ ID NO:57), GSGSGSGS (SEQ ID NO:58), GSGSGNGS (SEQ ID NO:59), GGSGSGSG (SEQ ID NO:60), GGSGSG (SEQ ID NO:61), GGSG (SEQ ID NO:62), GGSGNGSG (SEQ ID NO:63), GGNGSGSG (SEQ ID NO:64) and GGNGSG (SEQ ID NO:65). More particularly, the peptide linker is selected from (G4S)₁ or GGGGS (SEQ ID NO:51), (G₄S)₂ or GGGGSGGGGS (SEQ ID NO:52), (G₄S)₃ or GGGGSGGGGSGGGGS (SEQ ID NO:54) and (G₄S)₄ or GGGGSGGGGSGGGGSGGGGS (SEQ ID NO:56). Most particularly, the ectodomain of the costimulatory TNF family ligand or a fragment thereof and the huCMP trimerization domain are connected by a peptide linker of SEQ ID NO:56.

In a further aspect, provided is a trimeric costimulatory TNF family ligand-containing antigen binding molecule comprising three fusion polypeptides as described before, wherein the moiety capable of specific binding to a target cell antigen is fused at the C-terminal amino acid to the N-terminal amino acid of the trimerization domain, optionally through a peptide linker. In a particular aspect, the trimerization domain and the moiety capable of specific binding to a target cell antigen are connected through a peptide linker.

In particular, the peptide linker is a peptide selected from the group consisting of GGGGS (SEQ ID NO:51), GGGGSGGGGS (SEQ ID NO:52), SGGGGSGGGG (SEQ ID NO:53), (G₄S)₃ or GGGGSGGGGSGGGGS (SEQ ID NO:54), GGGGSGGGGSGGGG or G4(SG4)₂ (SEQ ID NO:55), and (G₄S)₄ or GGGGSGGGGSGGGGSGGGGS (SEQ ID NO:56), but also include the sequences GSPGSSSSGS (SEQ ID NO:57), GSGSGSGS (SEQ ID NO:58), GSGSGNGS (SEQ ID NO:59), GGSGSGSG (SEQ ID NO:60), GGSGSG (SEQ ID NO:61), GGSG (SEQ ID NO:62), GGSGNGSG (SEQ ID NO:63), GGNGSGSG (SEQ ID NO:64) and GGNGSG (SEQ ID NO:65). More particularly, the peptide linker is selected from (G₄S)₁ or GGGGS (SEQ ID NO:51), (G₄S)₂ or GGGGSGGGGS (SEQ ID NO:52), (G₄S)₃ or GGGGSGGGGSGGGGS (SEQ ID NO:54) and (G₄S)₄ or GGGGSGGGGSGGGGSGGGGS (SEQ ID NO:56). Most particularly, the huCMP trimerization domain and the moiety capable of specific binding to a target cell antigen are connected by a peptide linker of SEQ ID NO:52.

In another aspect, the invention provides a trimeric costimulatory TNF family ligand-containing antigen binding molecule comprising three fusion polypeptides as described before, wherein the moiety capable of specific binding to a target cell antigen is selected from the group consisting of is selected from the group consisting of an antibody, an antibody fragment and a scaffold antigen binding protein. In one aspect, the moiety capable of specific binding to a target cell antigen is selected from the group consisting of an antibody fragment, a Fab molecule, a crossover Fab molecule, a single chain Fab molecule, a Fv molecule, a scFv molecule, a single domain antibody, an aVH and a scaffold antigen binding protein.

In one aspect, the invention provides a trimeric costimulatory TNF family ligand-containing antigen binding molecule as defined herein before, wherein the moiety capable of specific binding to a target cell antigen is an antibody fragment. In particular, the antibody fragment is selected from the group consisting of a Fab molecule, a crossover Fab molecule, a single chain Fab molecule, a Fv molecule, a scFv molecule, a single domain antibody, and aVH. In one aspect, the moiety capable of specific binding to a target cell antigen is a single chain Fab molecule. In one aspect, the moiety capable of specific binding to a target cell antigen is a single domain antibody or an aVH.

In a further aspect, the invention provides a trimeric costimulatory TNF family ligand-containing antigen binding molecule as defined herein before, wherein the moiety capable of specific binding to a target cell antigen is a scaffold antigen binding protein. In particular, the moiety capable of specific binding to a target cell antigen can be a specifically designed ankyrin repeat protein.

In a particular aspect, the invention is concerned with a trimeric costimulatory TNF family ligand-containing antigen binding molecule as defined herein before, wherein the moiety capable of specific binding to a target cell antigen is a Fab molecule capable of specific binding to a target cell antigen. Thus, the invention provides a TNF family ligand trimer-containing antigen binding molecule comprising (a) an ectodomain of a costimulatory TNF family ligand or a fragment thereof, (b) a trimerization domain derived from human cartilage matrix protein (huCMP) of amino acid sequence of SEQ ID NO:1, and (c) a Fab molecule capable of specific binding to a target cell antigen.

In a further aspect, said Fab molecule is fused at the C-terminal amino acid of the CH1 domain to the N-terminal amino acid of the trimerization domain, optionally through a peptide linker as defined above.

In a further aspect, provided is a trimeric costimulatory TNF family ligand-containing antigen binding molecule comprising three fusion polypeptides as described before, wherein the target cell antigen is selected from the group consisting of Fibroblast Activation Protein (FAP), Melanoma-associated Chondroitin Sulfate Proteoglycan (MCSP), Epidermal Growth Factor Receptor (EGFR), Carcinoembryonic Antigen (CEA), CD19, CD20 and CD33. In a particular aspect, the target cell antigen is Fibroblast Activation Protein (FAP).

In a further aspect, provided is a trimeric costimulatory TNF family ligand-containing antigen binding molecule comprising three fusion polypeptides as described before, wherein the moiety capable of specific binding to FAP comprises

(a) a VH domain comprising (i) CDR-H1 comprising the amino acid sequence of SEQ ID NO:14, (ii) CDR-H2 comprising the amino acid sequence of SEQ ID NO:15 and (iii) CDR-H3 comprising the amino acid sequence of SEQ ID NO:16, and a VL domain comprising (iv) CDR-L1 comprising the amino acid sequence of SEQ ID NO:17, (v) CDR-L2 comprising the amino acid sequence of SEQ ID NO:18 and (vi) CDR-L3 comprising the amino acid sequence of SEQ ID NO:19 or
(b) a VH domain comprising (i) CDR-H1 comprising the amino acid sequence of SEQ ID NO:20, (ii) CDR-H2 comprising the amino acid sequence of SEQ ID NO:21 and (iii) CDR-H3 comprising the amino acid sequence of SEQ ID NO:22, and a VL domain comprising (iv) CDR-L1 comprising the amino acid sequence of SEQ ID NO:23, (v) CDR-L2 comprising the amino acid sequence of SEQ ID NO:24 and (vi) CDR-L3 comprising the amino acid sequence of SEQ ID NO:25.

In a further aspect, the moiety capable of specific binding to FAP comprises a heavy chain variable region comprising an amino acid sequence that is at least about 95%, 96%, 97%, 98%, 99% or 100% identical to the amino acid sequence of SEQ ID NO:26 and a light chain variable region comprising an amino acid sequence that is at least about 95%, 96%, 97%, 98%, 99% or 100% identical to the amino acid sequence of SEQ ID NO:27.

In another aspect, the moiety capable of specific binding to FAP comprises a heavy chain variable region comprising an amino acid sequence that is at least about 95%, 96%, 97%, 98%, 99% or 100% identical to the amino acid sequence of SEQ ID NO:28 and a light chain variable region comprising an amino acid sequence that is at least about 95%, 96%, 97%, 98%, 99% or 100% identical to the amino acid sequence of SEQ ID NO:29.

In a further aspect, the moiety capable of specific binding to FAP comprises

(a) a VH domain comprising the amino acid sequence of SEQ ID NO:26 and a VL domain comprising the amino acid sequence of SEQ ID NO:27, or (b) a VH domain comprising the amino acid sequence of SEQ ID NO:28 and a VL domain comprising the amino acid sequence of SEQ ID NO:29.

In a further aspect, provided is a trimeric costimulatory TNF family ligand-containing antigen binding molecule comprising three fusion polypeptides as described before, wherein said three fusion polypeptides are identical.

In another aspect, the invention provides a fusion polypeptide comprising (a) an ectodomain of a costimulatory TNF family ligand or a fragment thereof and (b) a trimerization domain derived from human cartilage matrix protein (huCMP) of the amino acid sequence of SEQ ID NO:2, wherein said trimerization domain is capable of mediating stable association of said fusion polypeptide with two further such fusion polypeptides and (c) a moiety capable of specific binding to a target cell antigen.

In yet a further aspect, provided is a fusion polypeptide, wherein the fusion polypeptide comprises

(a) an ectodomain of a TNF family ligand comprising the amino acid sequence selected from the group consisting of SEQ ID NO:3, SEQ ID NO:4, SEQ ID NO:5, SEQ ID NO:6, SEQ ID NO:7, SEQ ID NO:8, SEQ ID NO:9 and SEQ ID NO:10,
(b) a trimerization domain comprising the amino acid sequence of SEQ ID NO:2 and
(c) a VH domain comprising (i) CDR-H1 comprising the amino acid sequence of SEQ ID NO:14, (ii) CDR-H2 comprising the amino acid sequence of SEQ ID NO:15 and (iii) CDR-H3 comprising the amino acid sequence of SEQ ID NO:16 or a VH domain comprising (i) CDR-H1 comprising the amino acid sequence of SEQ ID NO:20, (ii) CDR-H2 comprising the amino acid sequence of SEQ ID NO:21 and (iii) CDR-H3 comprising the amino acid sequence of SEQ ID NO:22.

In a particular aspect, provided is a fusion polypeptide comprising the amino acid sequence of SEQ ID NO:71.

In another aspect, provided is a fusion polypeptide, wherein the fusion polypeptide comprises

(a) an ectodomain of a TNF family ligand comprising the amino acid sequence selected from the group consisting of SEQ ID NO:11, SEQ ID NO:12, and SEQ ID NO:13,
(b) a trimerization domain comprising the amino acid sequence of SEQ ID NO:2 and
(c) a VH domain comprising (i) CDR-H1 comprising the amino acid sequence of SEQ ID NO:14, (ii) CDR-H2 comprising the amino acid sequence of SEQ ID NO:15 and (iii) CDR-H3 comprising the amino acid sequence of SEQ ID NO:16 or a VH domain comprising (i) CDR-H1 comprising the amino acid sequence of SEQ ID NO:20, (ii) CDR-H2 comprising the amino acid sequence of SEQ ID NO:21 and (iii) CDR-H3 comprising the amino acid sequence of SEQ ID NO:22.

In a particular aspect, provided is a fusion polypeptide comprising the amino acid sequence of SEQ ID NO:72.

### Polynucleotides

The invention further provides isolated polynucleotides encoding a trimeric costimulatory TNF family ligand-containing antigen binding molecule as described herein or a fragment thereof.

The isolated polynucleotides encoding trimeric costimulatory TNF family ligand-containing antigen binding molecules of the invention may be expressed as a single polynucleotide that encodes the entire antigen binding molecule or as multiple (e.g., two or more) polynucleotides that are co-expressed. Polypeptides encoded by polynucleotides that are co-expressed may associate through, e.g., disulfide bonds or other means to form a functional antigen binding molecule. For example, the light chain portion of a moiety capable of specific binding to a target cell antigen may be encoded by a separate polynucleotide from the heavy chain portion of the capable of specific binding to a target cell antigen. When co-expressed, the heavy chain polypeptides will associate with the light chain polypeptides to form the moiety capable of specific binding to a target cell antigen.

In a particular aspect, the invention relates to an isolated polynucleotide encoding a fusion polypeptide as described herein before. The invention thus relates to a polynucleotide encoding a fusion polypeptide comprising (a) an ectodomain of a TNF family ligand comprising the amino acid sequence selected from the group consisting of SEQ ID NO:3, SEQ ID NO:4, SEQ ID NO:5, SEQ ID NO:6, SEQ ID NO:7, SEQ ID NO:8, SEQ ID NO:9 and SEQ ID NO:10, (b) a trimerization domain comprising the amino acid sequence of SEQ ID NO:2 and (c) a VH domain comprising (i) CDR-H1 comprising the amino acid sequence of SEQ ID NO:14, (ii) CDR-H2 comprising the amino acid sequence of SEQ ID NO:15 and (iii) CDR-H3 comprising the amino acid sequence of SEQ ID NO:16 or a VH domain comprising (i) CDR-H1 comprising the amino acid sequence of SEQ ID NO:20, (ii) CDR-H2 comprising the amino acid sequence of SEQ ID NO:21 and (iii) CDR-H3 comprising the amino acid sequence of SEQ ID NO:22.

In a particular aspect, provided is a polynucleotide encoding a fusion polypeptide comprising the amino acid sequence of SEQ ID NO:71. More particularly, provided is a polynucleotide comprising the sequence of SEQ ID NO:73.

In another aspect, provided is a polynucleotide encoding a fusion polypeptide comprising (a) an ectodomain of a TNF family ligand comprising the amino acid sequence selected from the group consisting of SEQ ID NO:11, SEQ ID NO:12, and SEQ ID NO:13, (b) a trimerization domain comprising the amino acid sequence of SEQ ID NO:2 and (c) a VH domain comprising (i) CDR-H1 comprising the amino acid sequence of SEQ ID NO:14, (ii) CDR-H2 comprising the amino acid sequence of SEQ ID NO:15 and (iii) CDR-H3 comprising the amino acid sequence of SEQ ID NO:16 or a VH domain comprising (i) CDR-H1 comprising the amino acid sequence of SEQ ID NO:20, (ii) CDR-H2 comprising the amino acid sequence of SEQ ID NO:21 and (iii) CDR-H3 comprising the amino acid sequence of SEQ ID NO:22.

In a particular aspect, provided is a polynucleotide encoding a fusion polypeptide comprising the amino acid sequence of SEQ ID NO:72. More particularly, provided is a polynucleotide comprising the sequence of SEQ ID NO:74.

In certain embodiments the polynucleotide or nucleic acid is DNA. In other embodiments, a polynucleotide of the present invention is RNA, for example, in the form of messenger RNA (mRNA). RNA of the present invention may be single stranded or double stranded.

According to another aspect of the invention, there is provided an isolated polynucleotide encoding a trimeric costimulatory TNF family ligand-containing antigen binding molecule as defined herein before or a fusion polypeptide as described herein before. The invention further provides a vector, particularly an expression vector, comprising the isolated polynucleotide of the invention and a host cell comprising the isolated polynucleotide or the vector of the invention. In some embodiments the host cell is a eukaryotic cell, particularly a mammalian cell.

In another aspect, provided is a method for producing the trimeric costimulatory TNF family ligand-containing antigen binding molecule of the invention, comprising the steps of (i) culturing the host cell of the invention under conditions suitable for expression of said antigen binding molecule, and (ii) isolating said trimeric antigen binding molecule. The invention also encompasses a trimeric costimulatory TNF family ligand-containing antigen binding molecule produced by the method of the invention.

### Recombinant Methods

Trimeric costimulatory TNF family ligand-containing antigen binding molecules of the invention may be obtained, for example, by solid-state peptide synthesis (e.g. Merrifield solid phase synthesis) or recombinant production. For recombinant production one or more polynucleotide encoding the TNF family ligand trimer-containing antigen binding molecule or polypeptide fragments thereof, e.g., as described above, is isolated and inserted into one or more vectors for further cloning and/or expression in a host cell. Such polynucleotide may be readily isolated and sequenced using conventional procedures. In one aspect of the invention, a vector, preferably an expression vector, comprising one or more of the polynucleotides of the invention is provided. Methods which are well known to those skilled in the art can be used to construct expression vectors containing the coding sequence of the trimeric costimulatory TNF family ligand-containing antigen binding molecule (fragment) along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y. (1989). The expression vector can be part of a plasmid, virus, or may be a nucleic acid fragment. The expression vector includes an expression cassette into which the polynucleotide encoding the trimeric costimulatory TNF family ligand-containing antigen binding molecule or polypeptide fragments thereof (i.e. the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g. on a single vector, or in separate polynucleotide constructs, e.g. on separate (different) vectors. Furthermore, any vector may contain a single coding region, or may comprise two or more coding regions, e.g. a vector of the present invention may encode one or more polypeptides, which are post- or co-translationally separated into the final proteins via proteolytic cleavage. In addition, a vector, polynucleotide, or nucleic acid of the invention may encode heterologous coding regions, either fused or unfused to a polynucleotide encoding the trimeric costimulatory TNF family ligand-containing antigen binding molecule of the invention or polypeptide fragments thereof, or variants or derivatives thereof. Heterologous coding regions include without limitation specialized elements or motifs, such as a secretory signal peptide or a heterologous functional domain. An operable association is when a coding region for a gene product, e.g. a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription.

Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g. the immediate early promoter, in conjunction with intron-A), simian virus 40 (e.g. the early promoter), and retroviruses (such as, e.g. Rous sarcoma virus). Other transcription control regions include those derived from vertebrate genes such as actin, heat shock protein, bovine growth hormone and rabbit â-globin, as well as other sequences capable of controlling gene expression in eukaryotic cells. Additional suitable transcription control regions include tissue-specific promoters and enhancers as well as inducible promoters (e.g. promoters inducible tetracyclins). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

Polynucleotide and nucleic acid coding regions of the present invention may be associated with additional coding regions which encode secretory or signal peptides, which direct the secretion of a polypeptide encoded by a polynucleotide of the present invention. For example, if secretion of the trimeric costimulatory TNF family ligand-containing antigen binding molecule or polypeptide fragments thereof is desired, DNA encoding a signal sequence may be placed upstream of the nucleic acid encoding a trimeric costimulatory TNF family ligand-containing antigen binding molecule of the invention or polypeptide fragments thereof. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the translated polypeptide to produce a secreted or “mature” form of the polypeptide. In certain embodiments, the native signal peptide, e.g. an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

DNA encoding a short protein sequence that could be used to facilitate later purification (e.g. a histidine tag) or assist in labeling the fusion protein may be included within or at the ends of the polynucleotide encoding a trimeric costimulatory TNF family ligand-containing antigen binding molecule of the invention or polypeptide fragments thereof.

In a further aspect of the invention, a host cell comprising one or more polynucleotides of the invention is provided. In certain embodiments a host cell comprising one or more vectors of the invention is provided. The polynucleotides and vectors may incorporate any of the features, singly or in combination, described herein in relation to polynucleotides and vectors, respectively. In one aspect, a host cell comprises (e.g. has been transformed or transfected with) a vector comprising a polynucleotide that encodes (part of) a trimeric costimulatory TNF family ligand-containing antigen binding molecule of the invention of the invention. As used herein, the term “host cell” refers to any kind of cellular system which can be engineered to generate the fusion proteins of the invention or fragments thereof. Host cells suitable for replicating and for supporting expression of antigen binding molecules are well known in the art. Such cells may be transfected or transduced as appropriate with the particular expression vector and large quantities of vector containing cells can be grown for seeding large scale fermenters to obtain sufficient quantities of the antigen binding molecule for clinical applications. Suitable host cells include prokaryotic microorganisms, such as E. coli, or various eukaryotic cells, such as Chinese hamster ovary cells (CHO), insect cells, or the like. For example, polypeptides may be produced in bacteria in particular when glycosylation is not needed. After expression, the polypeptide may be isolated from the bacterial cell paste in a soluble fraction and can be further purified. In addition to prokaryotes, eukaryotic microbes such as filamentous fungi or yeast are suitable cloning or expression hosts for polypeptide-encoding vectors, including fungi and yeast strains whose glycosylation pathways have been “humanized”, resulting in the production of a polypeptide with a partially or fully human glycosylation pattern. See Gerngross, Nat Biotech 22, 1409-1414 (2004), and Li et al., Nat Biotech 24, 210-215 (2006).

Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See e.g. U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham et al., J Gen Virol 36, 59 (1977)), baby hamster kidney cells (BHK), mouse sertoli cells (TM4 cells as described, e.g., in Mather, Biol Reprod 23, 243-251 (1980)), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells (as described, e.g., in Mather et al., Annals N.Y. Acad Sci 383, 44-68 (1982)), MRC 5 cells, and FS4 cells. Other useful mammalian host cell lines include Chinese hamster ovary (CHO) cells, including dhfr− CHO cells (Urlaub et al., Proc Natl Acad Sci USA 77, 4216 (1980)); and myeloma cell lines such as YO, NS0, P3X63 and Sp2/0. For a review of certain mammalian host cell lines suitable for protein production, see, e.g., Yazaki and Wu, Methods in Molecular Biology, Vol. 248 (B. K. C. Lo, ed., Humana Press, Totowa, N.J.), pp. 255-268 (2003). Host cells include cultured cells, e.g., mammalian cultured cells, yeast cells, insect cells, bacterial cells and plant cells, to name only a few, but also cells comprised within a transgenic animal, transgenic plant or cultured plant or animal tissue. In one embodiment, the host cell is a eukaryotic cell, preferably a mammalian cell, such as a Chinese Hamster Ovary (CHO) cell, a human embryonic kidney (HEK) cell or a lymphoid cell (e.g., Y0, NS0, Sp20 cell). Standard technologies are known in the art to express foreign genes in these systems. Cells expressing a polypeptide comprising either the heavy or the light chain of an antigen binding domain, may be engineered so as to also express the other of the immunoglobulin chains such that the expressed product is an antigen binding domain that has both a heavy and a light chain.

In another aspect, provided is a method for producing the trimeric costimulatory TNF family ligand-containing antigen binding molecule of the invention, comprising the steps of (i) culturing the host cell of the invention under conditions suitable for expression of said antigen binding molecule, and (ii) isolating said trimeric antigen binding molecule form the host cell or host cell culture medium.

The components of the trimeric antigen binding molecule are genetically fused to each other. Trimeric antigen binding molecules can be designed such that its components are fused directly to each other or indirectly through a linker sequence. The composition and length of the linker may be determined in accordance with methods well known in the art and may be tested for efficacy. Examples of linker sequences between different components of trimeric antigen binding molecules are found in the sequences provided herein. Additional sequences may also be included to incorporate a cleavage site to separate the individual components of the fusion if desired, for example an endopeptidase recognition sequence.

In certain embodiments the moieties capable of specific binding to a target cell antigen (e.g. Fab fragments) forming part of the antigen binding molecule comprise at least an immunoglobulin variable region capable of binding to an antigen. Variable regions can form part of and be derived from naturally or non-naturally occurring antibodies and fragments thereof. Methods to produce polyclonal antibodies and monoclonal antibodies are well known in the art (see e.g. Harlow and Lane, “Antibodies, a laboratory manual”, Cold Spring Harbor Laboratory, 1988). Non-naturally occurring antibodies can be constructed using solid phase-peptide synthesis, can be produced recombinantly (e.g. as described in U.S. Pat. No. 4,186,567) or can be obtained, for example, by screening combinatorial libraries comprising variable heavy chains and variable light chains (see e.g. U.S. Pat. No. 5,969,108 to McCafferty).

Any animal species of immunoglobulin can be used in the invention. Non-limiting immunoglobulins useful in the present invention can be of murine, primate, or human origin. If the fusion protein is intended for human use, a chimeric form of immunoglobulin may be used wherein the constant regions of the immunoglobulin are from a human. A humanized or fully human form of the immunoglobulin can also be prepared in accordance with methods well known in the art (see e. g. U.S. Pat. No. 5,565,332 to Winter). Humanization may be achieved by various methods including, but not limited to (a) grafting the non-human (e.g., donor antibody) CDRs onto human (e.g. recipient antibody) framework and constant regions with or without retention of critical framework residues (e.g. those that are important for retaining good antigen binding affinity or antibody functions), (b) grafting only the non-human specificity-determining regions (SDRs or a-CDRs; the residues critical for the antibody-antigen interaction) onto human framework and constant regions, or (c) transplanting the entire non-human variable domains, but “cloaking” them with a human-like section by replacement of surface residues. Humanized antibodies and methods of making them are reviewed, e.g., in Almagro and Fransson, Front Biosci 13, 1619-1633 (2008), and are further described, e.g., in Riechmann et al., Nature 332, 323-329 (1988); Queen et al., Proc Natl Acad Sci USA 86, 10029-10033 (1989); U.S. Pat. Nos. 5,821,337, 7,527,791, 6,982,321, and 7,087,409; Jones et al., Nature 321, 522-525 (1986); Morrison et al., Proc Natl Acad Sci 81, 6851-6855 (1984); Morrison and Oi, Adv Immunol 44, 65-92 (1988); Verhoeyen et al., Science 239, 1534-1536 (1988); Padlan, Molec Immun 31(3), 169-217 (1994); Kashmiri et al., Methods 36, 25-34 (2005) (describing SDR (a-CDR) grafting); Padlan, Mol Immunol 28, 489-498 (1991) (describing “resurfacing”); Dall'Acqua et al., Methods 36, 43-60 (2005) (describing “FR shuffling”); and Osbourn et al., Methods 36, 61-68 (2005) and Klimka et al., Br J Cancer 83, 252-260 (2000) (describing the “guided selection” approach to FR shuffling). Particular immunoglobulins according to the invention are human immunoglobulins. Human antibodies and human variable regions can be produced using various techniques known in the art. Human antibodies are described generally in van Dijk and van de Winkel, Curr Opin Pharmacol 5, 368-74 (2001) and Lonberg, Curr Opin Immunol 20, 450-459 (2008). Human variable regions can form part of and be derived from human monoclonal antibodies made by the hybridoma method (see e.g. Monoclonal Antibody Production Techniques and Applications, pp. 51-63 (Marcel Dekker, Inc., New York, 1987)). Human antibodies and human variable regions may also be prepared by administering an immunogen to a transgenic animal that has been modified to produce intact human antibodies or intact antibodies with human variable regions in response to antigenic challenge (see e.g. Lonberg, Nat Biotech 23, 1117-1125 (2005). Human antibodies and human variable regions may also be generated by isolating Fv clone variable region sequences selected from human-derived phage display libraries (see e.g., Hoogenboom et al. in Methods in Molecular Biology 178, 1-37 (O'Brien et al., ed., Human Press, Totowa, N.J., 2001); and McCafferty et al., Nature 348, 552-554; Clackson et al., Nature 352, 624-628 (1991)). Phage typically display antibody fragments, either as single-chain Fv (scFv) fragments or as Fab fragments.

In certain aspects, the moieties capable of specific binding to a target cell antigen (e.g. Fab fragments) comprised in the antigen binding molecules of the present invention are engineered to have enhanced binding affinity according to, for example, the methods disclosed in PCT publication WO 2012/020006 (see Examples relating to affinity maturation) or U.S. Pat. Appl. Publ. No. 2004/0132066. The ability of the antigen binding molecules of the invention to bind to a specific antigenic determinant can be measured either through an enzyme-linked immunosorbent assay (ELISA) or other techniques familiar to one of skill in the art, e.g. surface plasmon resonance technique (Liljeblad, et al., Glyco J 17, 323-329 (2000)), and traditional binding assays (Heeley, Endocr Res 28, 217-229 (2002)). Competition assays may be used to identify an antigen binding molecule that competes with a reference antibody for binding to a particular antigen. In certain embodiments, such a competing antigen binding molecule binds to the same epitope (e.g. a linear or a conformational epitope) that is bound by the reference antigen binding molecule. Detailed exemplary methods for mapping an epitope to which an antigen binding molecule binds are provided in Morris (1996) “Epitope Mapping Protocols”, in Methods in Molecular Biology vol. 66 (Humana Press, Totowa, N.J.). In an exemplary competition assay, immobilized antigen is incubated in a solution comprising a first labeled antigen binding molecule that binds to the antigen and a second unlabeled antigen binding molecule that is being tested for its ability to compete with the first antigen binding molecule for binding to the antigen. The second antigen binding molecule may be present in a hybridoma supernatant. As a control, immobilized antigen is incubated in a solution comprising the first labeled antigen binding molecule but not the second unlabeled antigen binding molecule. After incubation under conditions permissive for binding of the first antibody to the antigen, excess unbound antibody is removed, and the amount of label associated with immobilized antigen is measured. If the amount of label associated with immobilized antigen is substantially reduced in the test sample relative to the control sample, then that indicates that the second antigen binding molecule is competing with the first antigen binding molecule for binding to the antigen. See Harlow and Lane (1988) Antibodies: A Laboratory Manual ch.14 (Cold Spring Harbor Laboratory, Cold Spring Harbor, N.Y.).

Trimeric costimulatory TNF family ligand-containing antigen binding molecules of the invention prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the trimeric costimulatory TNF family ligand-containing antigen binding molecule binds. For example, for affinity chromatography purification of fusion proteins of the invention, a matrix with protein A or protein G may be used. Sequential Protein A or G affinity chromatography and size exclusion chromatography can be used to isolate an antigen binding molecule essentially as described in the Examples. The purity of the trimeric costimulatory TNF family ligand-containing antigen binding molecule or fragments thereof can be determined by any of a variety of well-known analytical methods including gel electrophoresis, high pressure liquid chromatography, and the like. For example, the trimeric costimulatory TNF family ligand-containing antigen binding molecule expressed as described in the Examples were shown to be intact and properly assembled as demonstrated by reducing and non-reducing SDS-PAGE.

The invention also encompasses a trimeric costimulatory TNF family ligand-containing antigen binding molecule produced by the methods of the invention.

### Assays

The trimeric costimulatory TNF family ligand-containing antigen binding molecules provided herein may be identified, screened for, or characterized for their physical/chemical properties and/or biological activities by various assays known in the art.

1. Affinity Assays

The affinity of the trimeric costimulatory TNF family ligand-containing antigen binding molecule provided herein for the corresponding TNF receptor can be determined in accordance with the methods set forth in the Examples by surface plasmon resonance (SPR), using standard instrumentation such as a BIAcore instrument (GE Healthcare), and receptors or target proteins such as may be obtained by recombinant expression. The affinity of the trimeric costimulatory TNF family ligand-containing antigen binding molecule for the target cell antigen can also be determined by surface plasmon resonance (SPR), using standard instrumentation such as a BIAcore instrument (GE Healthcare), and receptors or target proteins such as may be obtained by recombinant expression. A specific illustrative and exemplary embodiment for measuring binding affinity is described in Example 3.2. According to one aspect, K_{D} is measured by surface plasmon resonance using a BIACORE® T100 machine (GE Healthcare) at 25° C.

2. Binding Assays and Other Assays

Binding of the trimeric costimulatory TNF family ligand-containing antigen binding molecule provided herein to the corresponding receptor expressing cells may be evaluated using cell lines expressing the particular receptor or target antigen, for example by flow cytometry (FACS). In one aspect, fresh peripheral blood mononuclear cells (PBMCs) expressing the TNF receptor are used in the binding assay. These cells are used directly after isolation (naïve PMBCs) or after stimulation (activated PMBCs). In another aspect, activated mouse splenocytes (expressing the TNF receptor molecule) were used to demonstrate the binding of trimeric costimulatory TNF family ligand-containing antigen binding molecule of the invention to the corresponding TNF receptor expressing cells.

In a further aspect, cancer cell lines expressing the target cell antigen, for example FAP, were used to demonstrate the binding of the antigen binding molecules to the target cell antigen.

In another aspect, competition assays may be used to identify an antigen binding molecule that competes with a specific antibody or antigen binding molecule for binding to the target or TNF receptor, respectively. In certain embodiments, such a competing antigen binding molecule binds to the same epitope (e.g., a linear or a conformational epitope) that is bound by a specific anti-target antibody or a specific anti-TNF receptor antibody. Detailed exemplary methods for mapping an epitope to which an antibody binds are provided in Morris (1996) “Epitope Mapping Protocols,” in Methods in Molecular Biology vol. 66 (Humana Press, Totowa, N.J.).

3. Activity Assays

In one aspect, assays are provided for identifying trimeric costimulatory TNF family ligand-containing antigen binding molecules that bind to a specific target cell antigen and to a specific TNF receptor having biological activity. Biological activity may include, e.g., agonistic signalling through the TNF receptor on cells expressing the target cell antigen. TNF family ligand trimer-containing antigen binding molecules identified by the assays as having such biological activity in vitro are also provided. In particular, a reporter cell assay detecting NF-κB activation in Hela cells expressing human 4-1BB or human OX40 and co-cultured with FAP-expressing tumor cells is provided (see e.g. Example 5.1.2.).

In certain aspects, a trimeric costimulatory TNF family ligand-containing antigen binding molecules of the invention is tested for such biological activity. Assays for detecting the biological activity of the molecules of the invention are those described in Example 5 or Example 6.3. Furthermore, assays for detecting cell lysis (e.g. by measurement of LDH release), induced apoptosis kinetics (e.g. by measurement of Caspase 3/7 activity) or apoptosis (e.g. using the TUNEL assay) are well known in the art. In addition the biological activity of such complexes can be assessed by evaluating their effects on survival, proliferation and lymphokine secretion of various lymphocyte subsets such as NK cells, NKT-cells or γδ T-cells or assessing their capacity to modulate phenotype and function of antigen presenting cells such as dendritic cells, monocytes/macrophages or B-cells.

### Pharmaceutical Compositions, Formulations and Routes of Administration

In a further aspect, the invention provides pharmaceutical compositions comprising any of the trimeric costimulatory TNF family ligand-containing antigen binding molecules provided herein, e.g., for use in any of the below therapeutic methods. In one embodiment, a pharmaceutical composition comprises a trimeric costimulatory TNF family ligand-containing antigen binding molecule and at least one pharmaceutically acceptable excipient. In another embodiment, a pharmaceutical composition comprises any of the TNF family ligand trimer-containing antigen binding molecules provided herein and at least one additional therapeutic agent, e.g., as described below.

Pharmaceutical compositions of the present invention comprise a therapeutically effective amount of one or more trimeric costimulatory TNF family ligand-containing antigen binding molecule dissolved or dispersed in a pharmaceutically acceptable excipient. The phrases “pharmaceutical or pharmacologically acceptable” refers to molecular entities and compositions that are generally non-toxic to recipients at the dosages and concentrations employed, i.e. do not produce an adverse, allergic or other untoward reaction when administered to an animal, such as, for example, a human, as appropriate. The preparation of a pharmaceutical composition that contains at least one trimeric costimulatory TNF family ligand-containing antigen binding molecules and optionally an additional active ingredient will be known to those of skill in the art in light of the present disclosure, as exemplified by Remington's Pharmaceutical Sciences, 18th Ed. Mack Printing Company, 1990, incorporated herein by reference. In particular, the compositions are lyophilized formulations or aqueous solutions. As used herein, “pharmaceutically acceptable excipient” includes any and all solvents, buffers, dispersion media, coatings, surfactants, antioxidants, preservatives (e.g. antibacterial agents, antifungal agents), isotonic agents, salts, stabilizers and combinations thereof, as would be known to one of ordinary skill in the art.

Parenteral compositions include those designed for administration by injection, e.g. subcutaneous, intradermal, intralesional, intravenous, intraarterial intramuscular, intrathecal or intraperitoneal injection. For injection, the trimeric costimulatory TNF family ligand-containing antigen binding molecules of the invention may be formulated in aqueous solutions, preferably in physiologically compatible buffers such as Hanks' solution, Ringer's solution, or physiological saline buffer. The solution may contain formulatory agents such as suspending, stabilizing and/or dispersing agents. Alternatively, the fusion proteins may be in powder form for constitution with a suitable vehicle, e.g., sterile pyrogen-free water, before use. Sterile injectable solutions are prepared by incorporating the fusion proteins of the invention in the required amount in the appropriate solvent with various of the other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein. Suitable pharmaceutically acceptable excipients include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (18th Ed. Mack Printing Company, 1990). Sustained-release preparations may be prepared. Suitable examples of sustained-release preparations include semipermeable matrices of solid hydrophobic polymers containing the polypeptide, which matrices are in the form of shaped articles, e.g. films, or microcapsules. In particular embodiments, prolonged absorption of an injectable composition can be brought about by the use in the compositions of agents delaying absorption, such as, for example, aluminum monostearate, gelatin or combinations thereof.

Exemplary pharmaceutically acceptable excipients herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

In addition to the compositions described previously, the trimeric costimulatory TNF family ligand-containing antigen binding molecules may also be formulated as a depot preparation. Such long acting formulations may be administered by implantation (for example subcutaneously or intramuscularly) or by intramuscular injection. Thus, for example, the trimeric costimulatory TNF family ligand-containing antigen binding molecules may be formulated with suitable polymeric or hydrophobic materials (for example as emulsion in a pharmaceutically acceptable oil) or ion exchange resins, or as sparingly soluble derivatives, for example, as a sparingly soluble salt.

Pharmaceutical compositions comprising the trimeric costimulatory TNF family ligand-containing antigen binding molecules of the invention may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

The trimeric costimulatory TNF family ligand-containing antigen binding molecules may be formulated into a composition in a free acid or base, neutral or salt form. Pharmaceutically acceptable salts are salts that substantially retain the biological activity of the free acid or base. These include the acid addition salts, e.g. those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

The pharmaceutical compositions may also contain more than one active ingredients as necessary for the particular indication being treated, preferably those with complementary activities that do not adversely affect each other. Such active ingredients are suitably present in combination in amounts that are effective for the purpose intended. In one aspect, the pharmaceutical composition comprises a trimeric costimulatory TNF family ligand-containing antigen binding molecule and another active anti-cancer agent.

The formulations to be used for in vivo administration are generally sterile. Sterility may be readily accomplished, e.g., by filtration through sterile filtration membranes.

Therapeutic Methods and Compositions

Any of the trimeric costimulatory TNF family ligand-containing antigen binding molecules provided herein may be used in therapeutic methods. For use in therapeutic methods, TNF family ligand trimer-containing antigen binding molecules of the invention can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

In one aspect, trimeric costimulatory TNF family ligand-containing antigen binding molecules of the invention for use as a medicament are provided. In further aspects, trimeric costimulatory TNF family ligand-containing antigen binding molecules of the invention for use in treating a disease, in particular for use in the treatment of cancer, are provided. In certain embodiments, trimeric costimulatory TNF family ligand-containing antigen binding molecules of the invention for use in a method of treatment are provided. In one embodiment, the invention provides a trimeric costimulatory TNF family ligand-containing antigen binding molecule as described herein for use in the treatment of a disease in an individual in need thereof. In certain embodiments, the invention provides a trimeric costimulatory TNF family ligand-containing antigen binding molecule for use in a method of treating an individual having a disease comprising administering to the individual a therapeutically effective amount of the trimeric costimulatory TNF family ligand-containing antigen binding molecule. In certain embodiments the disease to be treated is cancer. In certain embodiments the disease to be treated is a proliferative disorder, particularly cancer. Examples of cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer. Other cell proliferation disorders that can be treated using a trimeric costimulatory TNF family ligand-containing antigen binding molecule of the present invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases. In certain embodiments the cancer is chosen from the group consisting of renal cell cancer, skin cancer, lung cancer, colorectal cancer, breast cancer, brain cancer, head and neck cancer. The subject, patient, or “individual” in need of treatment is typically a mammal, more specifically a human.

Also encompassed by the invention is the trimeric costimulatory TNF family ligand-containing antigen binding molecule of the invention, or the pharmaceutical composition of the invention, for use in up-regulating or prolonging cytotoxic T cell activity.

In a further aspect, the invention provides for the use of a trimeric costimulatory TNF family ligand-containing antigen binding molecule of the invention in the manufacture or preparation of a medicament for the treatment of a disease in an individual in need thereof. In one aspect, the medicament is for use in a method of treating a disease comprising administering to an individual having the disease a therapeutically effective amount of the medicament. In certain embodiments the disease to be treated is a proliferative disorder, particularly cancer. Examples of cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer. Other cell proliferation disorders that can be treated using a trimeric costimulatory TNF family ligand-containing antigen binding molecule of the present invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases. In certain embodiments the cancer is chosen from the group consisting of renal cell cancer, skin cancer, lung cancer, colorectal cancer, breast cancer, brain cancer, head and neck cancer. A skilled artisan readily recognizes that in many cases the trimeric costimulatory TNF family ligand-containing antigen binding molecule may not provide a cure but may only provide partial benefit. In some embodiments, a physiological change having some benefit is also considered therapeutically beneficial. Thus, in some embodiments, an amount of trimeric costimulatory TNF family ligand-containing antigen binding molecule that provides a physiological change is considered an “effective amount” or a “therapeutically effective amount”. In any of the above embodiments the individual is preferably a mammal, particularly a human.

In a further aspect, the invention provides a method for treating a disease in an individual, comprising administering to said individual a therapeutically effective amount of a trimeric costimulatory TNF family ligand-containing antigen binding molecule of the invention. In one embodiment a composition is administered to said individual, comprising a fusion protein of the invention in a pharmaceutically acceptable form. In certain embodiments the disease to be treated is a proliferative disorder. In a particular embodiment the disease is cancer. In certain embodiments the method further comprises administering to the individual a therapeutically effective amount of at least one additional therapeutic agent, e.g. an anti-cancer agent if the disease to be treated is cancer. An “individual” according to any of the above embodiments may be a mammal, preferably a human.

For the prevention or treatment of disease, the appropriate dosage of a trimeric costimulatory TNF family ligand-containing antigen binding molecule of the invention (when used alone or in combination with one or more other additional therapeutic agents) will depend on the type of disease to be treated, the route of administration, the body weight of the patient, the type of fusion protein, the severity and course of the disease, whether the fusion protein is administered for preventive or therapeutic purposes, previous or concurrent therapeutic interventions, the patient's clinical history and response to the fusion protein, and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

The trimeric costimulatory TNF family ligand-containing antigen binding molecule is suitably administered to the patient at one time or over a series of treatments. Depending on the type and severity of the disease, about 1 μg/kg to 15 mg/kg (e.g. 0.1 mg/kg-10 mg/kg) of TNF family ligand trimer-containing antigen binding molecule can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the fusion protein would be in the range from about 0.005 mg/kg to about 10 mg/kg. In other examples, a dose may also comprise from about 1 μg/kg body weight, about 5 μg/kg body weight, about 10 μg/kg body weight, about 50 μg/kg body weight, about 100 μg/kg body weight, about 200 μg/kg body weight, about 350 μg/kg body weight, about 500 μg/kg body weight, about 1 mg/kg body weight, about 5 mg/kg body weight, about 10 mg/kg body weight, about 50 mg/kg body weight, about 100 mg/kg body weight, about 200 mg/kg body weight, about 350 mg/kg body weight, about 500 mg/kg body weight, to about 1000 mg/kg body weight or more per administration, and any range derivable therein. In examples of a derivable range from the numbers listed herein, a range of about 5 mg/kg body weight to about 100 mg/kg body weight, about 5 μg/kg body weight to about 500 mg/kg body weight etc., can be administered, based on the numbers described above. Thus, one or more doses of about 0.5 mg/kg, 2.0 mg/kg, 5.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the trimeric costimulatory TNF family ligand-containing antigen binding molecule). An initial higher loading dose, followed by one or more lower doses may be administered. However, other dosage regimens may be useful. The progress of this therapy is easily monitored by conventional techniques and assays.

The trimeric costimulatory TNF family ligand-containing antigen binding molecule of the invention will generally be used in an amount effective to achieve the intended purpose. For use to treat or prevent a disease condition, the trimeric costimulatory TNF family ligand-containing antigen binding molecules of the invention, or pharmaceutical compositions thereof, are administered or applied in a therapeutically effective amount. Determination of a therapeutically effective amount is well within the capabilities of those skilled in the art, especially in light of the detailed disclosure provided herein.

For systemic administration, a therapeutically effective dose can be estimated initially from in vitro assays, such as cell culture assays. A dose can then be formulated in animal models to achieve a circulating concentration range that includes the IC50 as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the trimeric costimulatory TNF family ligand-containing antigen binding molecules which are sufficient to maintain therapeutic effect. Usual patient dosages for administration by injection range from about 0.1 to 50 mg/kg/day, typically from about 0.5 to 1 mg/kg/day. Therapeutically effective plasma levels may be achieved by administering multiple doses each day. Levels in plasma may be measured, for example, by HPLC.

In cases of local administration or selective uptake, the effective local concentration of the trimeric costimulatory TNF family ligand-containing antigen binding molecules may not be related to plasma concentration. One skilled in the art will be able to optimize therapeutically effective local dosages without undue experimentation.

A therapeutically effective dose of the trimeric costimulatory TNF family ligand-containing antigen binding molecules described herein will generally provide therapeutic benefit without causing substantial toxicity. Toxicity and therapeutic efficacy of a fusion protein can be determined by standard pharmaceutical procedures in cell culture or experimental animals. Cell culture assays and animal studies can be used to determine the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LD₅₀/ED₅₀. Trimeric costimulatory TNF family ligand-containing antigen binding molecules that exhibit large therapeutic indices are preferred. In one embodiment, the TNF family ligand trimer-containing antigen binding molecule according to the present invention exhibits a high therapeutic index. The data obtained from cell culture assays and animal studies can be used in formulating a range of dosages suitable for use in humans. The dosage lies preferably within a range of circulating concentrations that include the ED50 with little or no toxicity. The dosage may vary within this range depending upon a variety of factors, e.g., the dosage form employed, the route of administration utilized, the condition of the subject, and the like. The exact formulation, route of administration and dosage can be chosen by the individual physician in view of the patient's condition (see, e.g., Fingl et al., 1975, in: The Pharmacological Basis of Therapeutics, Ch. 1, p. 1, incorporated herein by reference in its entirety).

The attending physician for patients treated with the trimeric costimulatory TNF family ligand-containing antigen binding molecules of the invention will know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

Other Agents and Treatments

The trimeric costimulatory TNF family ligand-containing antigen binding molecules of the invention may be administered in combination with one or more other agents in therapy. For instance, a fusion protein of the invention may be co-administered with at least one additional therapeutic agent. The term “therapeutic agent” encompasses any agent that can be administered for treating a symptom or disease in an individual in need of such treatment. Such additional therapeutic agent may comprise any active ingredients suitable for the particular indication being treated, preferably those with complementary activities that do not adversely affect each other. In certain embodiments, an additional therapeutic agent is another anti-cancer agent.

Such other agents are suitably present in combination in amounts that are effective for the purpose intended. The effective amount of such other agents depends on the amount of fusion protein used, the type of disorder or treatment, and other factors discussed above. The trimeric costimulatory TNF family ligand-containing antigen binding molecules are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the trimeric costimulatory TNF family ligand-containing antigen binding molecule of the invention can occur prior to, simultaneously, and/or following, administration of the additional therapeutic agent and/or adjuvant.

Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper that is pierceable by a hypodermic injection needle). At least one active agent in the composition is a trimeric costimulatory TNF family ligand-containing antigen binding molecule of the invention.

The label or package insert indicates that the composition is used for treating the condition of choice. Moreover, the article of manufacture may comprise (a) a first container with a composition contained therein, wherein the composition comprises a trimeric costimulatory TNF family ligand-containing antigen binding molecule of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition.

Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

TABLE C
(Sequences):

| SEQ | | |
|---|---|---|
| ID NO: | Name | Sequence |
| 1 | Full length human CMP | MRVLSGTSLMLCSLLLLLQALCSPGLAPQSRGHLCR |
| | | TRPTDLVFVVDSSRSVRPVEFEKVKVFLSQVIESLD |
| | | VGPNATRVGMVNYASTVKQEFSLRAHVSKAALLQ |
| | | AVRRIQPLSTGTMTGLAIQFAITKAFGDAEGGRSRS |
| | | PDISKVVIVVTDGRPQDSVQDVSARARASGVELFAI |
| | | GVGSVDKATLRQIASEPQDEHVDYVESYSVIEKLSR |
| | | KFQEAFCVVSDLCATGDHDCEQVCISSPGSYTCAC |
| | | HEGFTLNSDGKTCNVCSGGGGSSATDLVFLIDGSKS |
| | | VRPENFELVKKFISQIVDTLDVSDKLAQVGLVQYSS |
| | | SVRQEFPLGRFHTKKDIKAAVRNMSYMEKGTMTG |
| | | AALKYLIDNSFTVSSGARPGAQKVGIVFTDGRSQDY |
| | | INDAAKKAKDLGFKMFAVGVGNAVEDELREIASEP |
| | | VAEHYFYTADFKTINQIGKKLQKKICVEEDPCACES |
| | | LVKFQAKVEGLLQALTRKLEAVSKRLAILENTVV |
| 2 | CMP trimerization domain | CACESLVKFQAKVEGLLQALTRKLEAVSKRLAILE |
| | | NTVV |
| 3 | Human (hu) 4-1BBL (71-254) | REGPELSPDDPAGLLDLRQGMFAQLVAQNVLLIDG |
| | | PLSWYSDPGLAGVSLTGGLSYKEDTKELVVAKAGV |
| | | YYVFFQLELRRVVAGEGSGSVSLALHLQPLRSAAG |
| | | AAALALTVDLPPASSEARNSAFGFQGRLLHLSAGQ |
| | | RLGVHLHTEARARHAWQLTQGATVLGLFRVTPEIP |
| | | AGLPSPRSE |
| 4 | hu 4-1BBL (85-254) | LDLRQGMFAQLVAQNVLLIDGPLSWYSDPGLAGVS |
| | | LTGGLSYKEDTKELVVAKAGVYYVFFQLELRRVV |
| | | AGEGSGSVSLALHLQPLRSAAGAAALALTVDLPPA |
| | | SSEARNSAFGFQGRLLHLSAGQRLGVHLHTEARAR |
| | | HAWQLTQGATVLGLFRVTPEIPAGLPSPRSE |
| 5 | hu 4-1BBL (80-254) | DPAGLLDLRQGMFAQLVAQNVLLIDGPLSWYSDPG |
| | | LAGVSLTGGLSYKEDTKELVVAKAGVYYVFFQLEL |
| | | RRVVAGEGSGSVSLALHLQPLRSAAGAAALALTVD |
| | | LPPASSEARNSAFGFQGRLLHLSAGQRLGVHLHTEA |
| | | RARHAWQLTQGATVLGLFRVTPEIPAGLPSPRSE |
| 6 | hu 4-1BBL (52-254) | PWAVSGARASPGSAASPRLREGPELSPDDPAGLLDL |
| | | RQGMFAQLVAQNVLLIDGPLSWYSDPGLAGVSLTG |
| | | GLSYKEDTKELVVAKAGVYYVFFQLELRRVVAGE |
| | | GSGSVSLALHLQPLRSAAGAAALALTVDLPPASSEA |
| | | RNSAFGFQGRLLHLSAGQRLGVHLHTEARARHAW |
| | | QLTQGATVLGLFRVTPEIPAGLPSPRSE |
| 7 | Human (hu) 4-1BBL (71-248) | REGPELSPDDPAGLLDLRQGMFAQLVAQNVLLIDG |
| | | PLSWYSDPGLAGVSLTGGLSYKEDTKELVVAKAGV |
| | | YYVFFQLELRRVVAGEGSGSVSLALHLQPLRSAAG |
| | | AAALALTVDLPPASSEARNSAFGFQGRLLHLSAGQ |
| | | RLGVHLHTEARARHAWQLTQGATVLGLFRVTPEIP |
| | | AGL |
| 8 | hu 4-1BBL (85-248) | LDLRQGMFAQLVAQNVLLIDGPLSWYSDPGLAGVS |
| | | LTGGLSYKEDTKELVVAKAGVYYVFFQLELRRVV |
| | | AGEGSGSVSLALHLQPLRSAAGAAALALTVDLPPA |
| | | SSEARNSAFGFQGRLLHLSAGQRLGVHLHTEARAR |
| | | HAWQLTQGATVLGLFRVTPEIPAGL |
| 9 | hu 4-1BBL (80-248) | DPAGLLDLRQGMFAQLVAQNVLLIDGPLSWYSDPG |
| | | LAGVSLTGGLSYKEDTKELVVAKAGVYYVFFQLEL |
| | | RRVVAGEGSGSVSLALHLQPLRSAAGAAALALTVD |
| | | LPPASSEARNSAFGFQGRLLHLSAGQRLGVHLHTEA |
| | | RARHAWQLTQGATVLGLFRVTPEIPAGL |
| 10 | hu 4-1BBL (52-248) | PWAVSGARASPGSAASPRLREGPELSPDDPAGLLDL |
| | | RQGMFAQLVAQNVLLIDGPLSWYSDPGLAGVSLTG |
| | | GLSYKEDTKELVVAKAGVYYVFFQLELRRVVAGE |
| | | GSGSVSLALHLQPLRSAAGAAALALTVDLPPASSEA |
| | | RNSAFGFQGRLLHLSAGQRLGVHLHTEARARHAW |
| | | QLTQGATVLGLFRVTPEIPAGL |
| 11 | hu OX40L (51-183) | QVSHRYPRIQSIKVQFTEYKKEKGFILTSQKEDEIMK |
| | | VQNNSVIINCDGFYLISLKGYFSQEVNISLHYQKDEE |
| | | PLFQLKKVRSVNSLMVASLTYKDKVYLNVTTDNTS |
| | | LDDFHVNGGELILIHQNPGEFCVL |
| 12 | hu OX40L (51-183) N90D, | QVSHRYPRIQSIKVQFTEYKKEKGFILTSQKEDEIMK |
| | N114D | VQDNSVIINCDGFYLISLKGYFSQEVDISLHYQKDEE |
| | | PLFQLKKVRSVNSLMVASLTYKDKVYLNVTTDNTS |
| | | LDDFHVNGGELILIHQNPGEFCVL |
| 13 | hu OX40L (52-183) | VSHRYPRIQSIKVQFTEYKKEKGFILTSQKEDEIMKV |
| | | QNNSVIINCDGFYLISLKGYFSQEVNISLHYQKDEEP |
| | | LFQLKKVRSVNSLMVASLTYKDKVYLNVTTDNTSL |
| | | DDFHVNGGELILIHQNPGEFCVL |
| 14 | FAP(28H1) CDR-H1 | SHAMS |
| 15 | FAP(28H1) CDR-H2 | AIWASGEQYYADSVKG |
| 16 | FAP(28H1) CDR-H3 | GWLGNFDY |
| 17 | FAP(28H1) CDR-L1 | RASQSVSRSYLA |
| 18 | FAP(28H1) CDR-L2 | GASTRAT |
| 19 | FAP(28H1) CDR-L3 | QQGQVIPPT |
| 20 | FAP(4B9) CDR-H1 | SYAMS |
| 21 | FAP(4B9) CDR-H2 | AIIGSGASTYYADSVKG |
| 22 | FAP(4B9) CDR-H3 | GWFGGFNY |
| 23 | FAP(4B9) CDR-L1 | RASQSVTSSYLA |
| 24 | FAP(4B9) CDR-L2 | VGSRRAT |
| 25 | FAP(4B9) CDR-L3 | QQGIMLPPT |
| 26 | FAP(28H1) VH | EVQLLESGGGLVQPGGSLRLSCAASGFTFSSHA |
| | | MSWVRQAPGKGLEWVSAIWASGEQYYADSV |
| | | KGRFTISRDNSKNTLYLQMNSLRAEDTAVYYC |
| | | AKGWLGNFDYWGQGTLVTVSS |
| 27 | FAP(28H1) VL | EIVLTQSPGTLSLSPGERATLSCRASQSVSRSYL |
| | | AWYQQKPGQAPRLLIIGASTRATGIPDRFSGSG |
| | | SGTDFTLTISRLEPEDFAVYYCQQGQVIPPTFGQ |
| | | GTKVEIK |
| 28 | FAP(4B9) VH | EVQLLESGGGLVQPGGSLRLSCAASGFTFSSYAMS |
| | | WVRQAPGKGLEWVSAIIGSGASTYYADSVKGRFTI |
| | | SRDNSKNTLYLQMNSLRAEDTAVYYCAKGWFGGF |
| | | NYWGQGTLVTVSS |
| 29 | FAP(4B9) VL | EIVLTQSPGTLSLSPGERATLSCRASQSVTSSYLAWY |
| | | QQKPGQAPRLLINVGSRRATGIPDRFSGSGSGTDFT |
| | | LTISRLEPEDFAVYYCQQGIMLPPTFGQGTKVEIK |
| 30 | Human (hu) FAP | UniProt no. Q12884 |
| 31 | hu FAP ectodomain + poly-lys- | RPSRVHNSEENTMRALTLKDILNGTFSYKTFFPNWI |
| | tag + his₆-tag | SGQEYLHQSADNNIVLYNIETGQSYTILSNRTMKSV |
| | | NASNYGLSPDRQFVYLESDYSKLWRYSYTATYYIY |
| | | DLSNGEFVRGNELPRPIQYLCWSPVGSKLAYVYQN |
| | | NIYLKQRPGDPPFQITFNGRENKIFNGIPDWVYEEE |
| | | MLATKYALWWSPNGKFLAYAEFNDTDIPVIAYSYY |
| | | GDEQYPRTINIPYPKAGAKNPVVRIFIIDTTYPAYVG |
| | | PQEVPVPAMIASSDYYFSWLTWVTDERVCLQWLK |
| | | RVQNVSVLSICDFREDWQTWDCPKTQEHIEESRTG |
| | | WAGGFFVSTPVFSYDAISYYKIFSDKDGYKHIHYIK |
| | | DTVENAIQITSGKWEAINIFRVTQDSLFYSSNEFEEY |
| | | PGRRNIYRISIGSYPPSKKCVTCHLRKERCQYYTASF |
| | | SDYAKYYALVCYGPGIPISTLHDGRTDQEIKILEENK |
| | | ELENALKNIQLPKEEIKKLEVDEITLWYKMILPPQFD |
| | | RSKKYPLLIQVYGGPCSQSVRSVFAVNWISYLASKE |
| | | GMVIALVDGRGTAFQGDKLLYAVYRKLGVYEVED |
| | | QITAVRKFIEMGFIDEKRIAIWGWSYGGYVSSLALA |
| | | SGTGLFKCGIAVAPVSSWEYYASVYTERFMGLPTK |
| | | DDNLEHYKNSTVMARAEYFRNVDYLLIHGTADDN |
| | | VHFQNSAQIAKALVNAQVDFQAMWYSDQNHGLSG |
| | | LSTNHLYTHMTHFLKQCFSLSDGKKKKKKGHHHH |
| | | HH |
| 32 | nucleotide sequence hu FAP | CGCCCTTCAAGAGTTCATAACTCTGAAGAAAATA |
| | ectodomain + poly-lys- | CAATGAGAGCACTCACACTGAAGGATATTTTAAA |
| | tag + his₆-tag | TGGAACATTTTCTTATAAAACATTTTTTCCAAACT |
| | | GGATTTCAGGACAAGAATATCTTCATCAATCTGC |
| | | AGATAACAATATAGTACTTTATAATATTGAAACA |
| | | GGACAATCATATACCATTTTGAGTAATAGAACCA |
| | | TGAAAAGTGTGAATGCTTCAAATTACGGCTTATC |
| | | ACCTGATCGGCAATTTGTATATCTAGAAAGTGAT |
| | | TATTCAAAGCTTTGGAGATACTCTTACACAGCAA |
| | | CATATTACATCTATGACCTTAGCAATGGAGAATT |
| | | TGTAAGAGGAAATGAGCTTCCTCGTCCAATTCAG |
| | | TATTTATGCTGGTCGCCTGTTGGGAGTAAATTAG |
| | | CATATGTCTATCAAAACAATATCTATTTGAAACA |
| | | AAGACCAGGAGATCCACCTTTTCAAATAACATTT |
| | | AATGGAAGAGAAAATAAAATATTTAATGGAATC |
| | | CCAGACTGGGTTTATGAAGAGGAAATGCTTGCTA |
| | | CAAAATATGCTCTCTGGTGGTCTCCTAATGGAAA |
| | | ATTTTTGGCATATGCGGAATTTAATGATACGGAT |
| | | ATACCAGTTATTGCCTATTCCTATTATGGCGATGA |
| | | ACAATATCCTAGAACAATAAATATTCCATACCCA |
| | | AAGGCTGGAGCTAAGAATCCCGTTGTTCGGATAT |
| | | TTATTATCGATACCACTTACCCTGCGTATGTAGGT |
| | | CCCCAGGAAGTGCCTGTTCCAGCAATGATAGCCT |
| | | CAAGTGATTATTATTTCAGTTGGCTCACGTGGGTT |
| | | ACTGATGAACGAGTATGTTTGCAGTGGCTAAAAA |
| | | GAGTCCAGAATGTTTCGGTCCTGTCTATATGTGA |
| | | CTTCAGGGAAGACTGGCAGACATGGGATTGTCCA |
| | | AAGACCCAGGAGCATATAGAAGAAAGCAGAACT |
| | | GGATGGGCTGGTGGATTCTTTGTTTCAACACCAG |
| | | TTTTCAGCTATGATGCCATTTCGTACTACAAAATA |
| | | TTTAGTGACAAGGATGGCTACAAACATATTCACT |
| | | ATATCAAAGACACTGTGGAAAATGCTATTCAAAT |
| | | TACAAGTGGCAAGTGGGAGGCCATAAATATATTC |
| | | AGAGTAACACAGGATTCACTGTTTTATTCTAGCA |
| | | ATGAATTTGAAGAATACCCTGGAAGAAGAAACA |
| | | TCTACAGAATTAGCATTGGAAGCTATCCTCCAAG |
| | | CAAGAAGTGTGTTACTTGCCATCTAAGGAAAGAA |
| | | AGGTGCCAATATTACACAGCAAGTTTCAGCGACT |
| | | ACGCCAAGTACTATGCACTTGTCTGCTACGGCCC |
| | | AGGCATCCCCATTTCCACCCTTCATGATGGACGC |
| | | ACTGATCAAGAAATTAAAATCCTGGAAGAAAAC |
| | | AAGGAATTGGAAAATGCTTTGAAAAATATCCAGC |
| | | TGCCTAAAGAGGAAATTAAGAAACTTGAAGTAG |
| | | ATGAAATTACTTTATGGTACAAGATGATTCTTCCT |
| | | CCTCAATTTGACAGATCAAAGAAGTATCCCTTGC |
| | | TAATTCAAGTGTATGGTGGTCCCTGCAGTCAGAG |
| | | TGTAAGGTCTGTATTTGCTGTTAATTGGATATCTT |
| | | ATCTTGCAAGTAAGGAAGGGATGGTCATTGCCTT |
| | | GGTGGATGGTCGAGGAACAGCTTTCCAAGGTGAC |
| | | AAACTCCTCTATGCAGTGTATCGAAAGCTGGGTG |
| | | TTTATGAAGTTGAAGACCAGATTACAGCTGTCAG |
| | | AAAATTCATAGAAATGGGTTTCATTGATGAAAAA |
| | | AGAATAGCCATATGGGGCTGGTCCTATGGAGGAT |
| | | ACGTTTCATCACTGGCCCTTGCATCTGGAACTGGT |
| | | CTTTTCAAATGTGGTATAGCAGTGGCTCCAGTCTC |
| | | CAGCTGGGAATATTACGCGTCTGTCTACACAGAG |
| | | AGATTCATGGGTCTCCCAACAAAGGATGATAATC |
| | | TTGAGCACTATAAGAATTCAACTGTGATGGCAAG |
| | | AGCAGAATATTTCAGAAATGTAGACTATCTTCTC |
| | | ATCCACGGAACAGCAGATGATAATGTGCACTTTC |
| | | AAAACTCAGCACAGATTGCTAAAGCTCTGGTTAA |
| | | TGCACAAGTGGATTTCCAGGCAATGTGGTACTCT |
| | | GACCAGAACCACGGCTTATCCGGCCTGTCCACGA |
| | | ACCACTTATACACCCACATGACCCACTTCCTAAA |
| | | GCAGTGTTTCTCTTTGTCAGACGGCAAAAAGAAA |
| | | AAGAAAAAGGGCCACCACCATCACCATCAC |
| 33 | mouse FAP | UniProt no. P97321 |
| 34 | Murine FAP | RPSRVYKPEGNTKRALTLKDILNGTFSYKTYFPNWI |
| | ectodomain + poly-lys- | SEQEYLHQSEDDNIVFYNIETRESYIILSNSTMKSVN |
| | tag + his₆-tag | ATDYGLSPDRQFVYLESDYSKLWRYSYTATYYIYD |
| | | LQNGEFVRGYELPRPIQYLCWSPVGSKLAYVYQNN |
| | | IYLKQRPGDPPFQITYTGRENRIFNGIPDWVYEEEML |
| | | ATKYALWWSPDGKFLAYVEFNDSDIPIIAYSYYGD |
| | | GQYPRTINIPYPKAGAKNPVVRVFIVDTTYPHHVGP |
| | | MEVPVPEMIASSDYYFSWLTWVSSERVCLQWLKR |
| | | VQNVSVLSICDFREDWHAWECPKNQEHVEESRTG |
| | | WAGGFFVSTPAFSQDATSYYKIFSDKDGYKHIHYIK |
| | | DTVENAIQITSGKWEAIYIFRVTQDSLFYSSNEFEGY |
| | | PGRRNIYRISIGNSPPSKKCVTCHLRKERCQYYTASF |
| | | SYKAKYYALVCYGPGLPISTLHDGRTDQEIQVLEEN |
| | | KELENSLRNIQLPKVEIKKLKDGGLTFWYKMILPPQ |
| | | FDRSKKYPLLIQVYGGPCSQSVKSVFAVNWITYLAS |
| | | KEGIVIALVDGRGTAFQGDKFLHAVYRKLGVYEVE |
| | | DQLTAVRKFIEMGFIDEERIAIWGWSYGGYVSSLAL |
| | | ASGTGLFKCGIAVAPVSSWEYYASIYSERFMGLPTK |
| | | DDNLEHYKNSTVMARAEYFRNVDYLLIHGTADDN |
| | | VHFQNSAQIAKALVNAQVDFQAMWYSDQNHGILS |
| | | GRSQNHLYTHMTHFLKQCFSLSDGKKKKKKGHHH |
| | | HHH |
| 35 | nucleotide sequence of | CGTCCCTCAAGAGTTTACAAACCTGAAGGAAACA |
| | murine FAP | CAAAGAGAGCTCTTACCTTGAAGGATATTTTAAA |
| | ectodomain + poly-lys- | TGGAACATTCTCATATAAAACATATTTTCCCAACT |
| | tag + his₆-tag | GGATTTCAGAACAAGAATATCTTCATCAATCTGA |
| | | GGATGATAACATAGTATTTTATAATATTGAAACA |
| | | AGAGAATCATATATCATTTTGAGTAATAGCACCA |
| | | TGAAAAGTGTGAATGCTACAGATTATGGTTTGTC |
| | | ACCTGATCGGCAATTTGTGTATCTAGAAAGTGAT |
| | | TATTCAAAGCTCTGGCGATATTCATACACAGCGA |
| | | CATACTACATCTACGACCTTCAGAATGGGGAATT |
| | | TGTAAGAGGATACGAGCTCCCTCGTCCAATTCAG |
| | | TATCTATGCTGGTCGCCTGTTGGGAGTAAATTAG |
| | | CATATGTATATCAAAACAATATTTATTTGAAACA |
| | | AAGACCAGGAGATCCACCTTTTCAAATAACTTAT |
| | | ACTGGAAGAGAAAATAGAATATTTAATGGAATA |
| | | CCAGACTGGGTTTATGAAGAGGAAATGCTTGCCA |
| | | CAAAATATGCTCTTTGGTGGTCTCCAGATGGAAA |
| | | ATTTTTGGCATATGTAGAATTTAATGATTCAGATA |
| | | TACCAATTATTGCCTATTCTTATTATGGTGATGGA |
| | | CAGTATCCTAGAACTATAAATATTCCATATCCAA |
| | | AGGCTGGGGCTAAGAATCCGGTTGTTCGTGTTTT |
| | | TATTGTTGACACCACCTACCCTCACCACGTGGGC |
| | | CCAATGGAAGTGCCAGTTCCAGAAATGATAGCCT |
| | | CAAGTGACTATTATTTCAGCTGGCTCACATGGGT |
| | | GTCCAGTGAACGAGTATGCTTGCAGTGGCTAAAA |
| | | AGAGTGCAGAATGTCTCAGTCCTGTCTATATGTG |
| | | ATTTCAGGGAAGACTGGCATGCATGGGAATGTCC |
| | | AAAGAACCAGGAGCATGTAGAAGAAAGCAGAAC |
| | | AGGATGGGCTGGTGGATTCTTTGTTTCGACACCA |
| | | GCTTTTAGCCAGGATGCCACTTCTTACTACAAAA |
| | | TATTTAGCGACAAGGATGGTTACAAACATATTCA |
| | | CTACATCAAAGACACTGTGGAAAATGCTATTCAA |
| | | ATTACAAGTGGCAAGTGGGAGGCCATATATATAT |
| | | TCCGCGTAACACAGGATTCACTGTTTTATTCTAGC |
| | | AATGAATTTGAAGGTTACCCTGGAAGAAGAAAC |
| | | ATCTACAGAATTAGCATTGGAAACTCTCCTCCGA |
| | | GCAAGAAGTGTGTTACTTGCCATCTAAGGAAAGA |
| | | AAGGTGCCAATATTACACAGCAAGTTTCAGCTAC |
| | | AAAGCCAAGTACTATGCACTCGTCTGCTATGGCC |
| | | CTGGCCTCCCCATTTCCACCCTCCATGATGGCCGC |
| | | ACAGACCAAGAAATACAAGTATTAGAAGAAAAC |
| | | AAAGAACTGGAAAATTCTCTGAGAAATATCCAGC |
| | | TGCCTAAAGTGGAGATTAAGAAGCTCAAAGACG |
| | | GGGGACTGACTTTCTGGTACAAGATGATTCTGCC |
| | | TCCTCAGTTTGACAGATCAAAGAAGTACCCTTTG |
| | | CTAATTCAAGTGTATGGTGGTCCTTGTAGCCAGA |
| | | GTGTTAAGTCTGTGTTTGCTGTTAATTGGATAACT |
| | | TATCTCGCAAGTAAGGAGGGGATAGTCATTGCCC |
| | | TGGTAGATGGTCGGGGCACTGCTTTCCAAGGTGA |
| | | CAAATTCCTGCATGCCGTGTATCGAAAACTGGGT |
| | | GTATATGAAGTTGAGGACCAGCTCACAGCTGTCA |
| | | GAAAATTCATAGAAATGGGTTTCATTGATGAAGA |
| | | AAGAATAGCCATATGGGGCTGGTCCTACGGAGGT |
| | | TATGTTTCATCCCTGGCCCTTGCATCTGGAACTGG |
| | | TCTTTTCAAATGTGGCATAGCAGTGGCTCCAGTCT |
| | | CCAGCTGGGAATATTACGCATCTATCTACTCAGA |
| | | GAGATTCATGGGCCTCCCAACAAAGGACGACAAT |
| | | CTCGAACACTATAAAAATTCAACTGTGATGGCAA |
| | | GAGCAGAATATTTCAGAAATGTAGACTATCTTCT |
| | | CATCCACGGAACAGCAGATGATAATGTGCACTTT |
| | | CAGAACTCAGCACAGATTGCTAAAGCTTTGGTTA |
| | | ATGCACAAGTGGATTTCCAGGCGATGTGGTACTC |
| | | TGACCAGAACCATGGTATATTATCTGGGCGCTCC |
| | | CAGAATCATTTATATACCCACATGACGCACTTCC |
| | | TCAAGCAATGCTTTTCTTTATCAGACGGCAAAAA |
| | | GAAAAAGAAAAAGGGCCACCACCATCACCATCAC |
| 36 | Cynomolgus FAP | RPPRVHNSEENTMRALTLKDILNGTFSYKTFFPNWI |
| | ectodomain + poly-lys- | SGQEYLHQSADNNIVLYNIETGQSYTILSNRTMKSV |
| | tag + his₆-tag | NASNYGLSPDRQFVYLESDYSKLWRYSYTATYYIY |
| | | DLSNGEFVRGNELPRPIQYLCWSPVGSKLAYVYQN |
| | | NIYLKQRPGDPPFQITFNGRENKIFNGIPDWVYEEE |
| | | MLATKYALWWSPNGKFLAYAEFNDTDIPVIAYSYY |
| | | GDEQYPRTINIPYPKAGAKNPFVRIFIIDTTYPAYVG |
| | | PQEVPVPAMIASSDYYFSWLTWVTDERVCLQWLK |
| | | RVQNVSVLSICDFREDWQTWDCPKTQEHIEESRTG |
| | | WAGGFFVSTPVFSYDAISYYKIFSDKDGYKHIHYIK |
| | | DTVENAIQITSGKWEAINIFRVTQDSLFYSSNEFEDY |
| | | PGRRNIYRISIGSYPPSKKCVTCHLRKERCQYYTASF |
| | | SDYAKYYALVCYGPGIPISTLHDGRTDQEIKILEENK |
| | | ELENALKNIQLPKEEIKKLEVDEITLWYKMILPPQFD |
| | | RSKKYPLLIQVYGGPCSQSVRSVFAVNWISYLASKE |
| | | GMVIALVDGRGTAFQGDKLLYAVYRKLGVYEVED |
| | | QITAVRKFIEMGFIDEKRIAIWGWSYGGYVSSLALA |
| | | SGTGLFKCGIAVAPVSSWEYYASVYTERFMGLPTK |
| | | DDNLEHYKNSTVMARAEYFRNVDYLLIHGTADDN |
| | | VHFQNSAQIAKALVNAQVDFQAMWYSDQNHGLSG |
| | | LSTNHLYTHMTHFLKQCFSLSDGKKKKKKGHHHH |
| | | HH |
| 37 | nucleotide sequence of | CGCCCTCCAAGAGTTCATAACTCTGAAGAAAATA |
| | cynomolgus FAP | CAATGAGAGCACTCACACTGAAGGATATTTTAAA |
| | ectodomain + poly-lys- | TGGGACATTTTCTTATAAAACATTTTTTCCAAACT |
| | tag + his₆-tag | GGATTTCAGGACAAGAATATCTTCATCAATCTGC |
| | | AGATAACAATATAGTACTTTATAATATTGAAACA |
| | | GGACAATCATATACCATTTTGAGTAACAGAACCA |
| | | TGAAAAGTGTGAATGCTTCAAATTATGGCTTATC |
| | | ACCTGATCGGCAATTTGTATATCTAGAAAGTGAT |
| | | TATTCAAAGCTTTGGAGATACTCTTACACAGCAA |
| | | CATATTACATCTATGACCTTAGCAATGGAGAATT |
| | | TGTAAGAGGAAATGAGCTTCCTCGTCCAATTCAG |
| | | TATTTATGCTGGTCGCCTGTTGGGAGTAAATTAG |
| | | CATATGTCTATCAAAACAATATCTATTTGAAACA |
| | | AAGACCAGGAGATCCACCTTTTCAAATAACATTT |
| | | AATGGAAGAGAAAATAAAATATTTAATGGAATC |
| | | CCAGACTGGGTTTATGAAGAGGAAATGCTTGCTA |
| | | CAAAATATGCTCTCTGGTGGTCTCCTAATGGAAA |
| | | ATTTTTGGCATATGCGGAATTTAATGATACAGAT |
| | | ATACCAGTTATTGCCTATTCCTATTATGGCGATGA |
| | | ACAATATCCCAGAACAATAAATATTCCATACCCA |
| | | AAGGCCGGAGCTAAGAATCCTTTTGTTCGGATAT |
| | | TTATTATCGATACCACTTACCCTGCGTATGTAGGT |
| | | CCCCAGGAAGTGCCTGTTCCAGCAATGATAGCCT |
| | | CAAGTGATTATTATTTCAGTTGGCTCACGTGGGTT |
| | | ACTGATGAACGAGTATGTTTGCAGTGGCTAAAAA |
| | | GAGTCCAGAATGTTTCGGTCTTGTCTATATGTGAT |
| | | TTCAGGGAAGACTGGCAGACATGGGATTGTCCAA |
| | | AGACCCAGGAGCATATAGAAGAAAGCAGAACTG |
| | | GATGGGCTGGTGGATTCTTTGTTTCAACACCAGTT |
| | | TTCAGCTATGATGCCATTTCATACTACAAAATATT |
| | | TAGTGACAAGGATGGCTACAAACATATTCACTAT |
| | | ATCAAAGACACTGTGGAAAATGCTATTCAAATTA |
| | | CAAGTGGCAAGTGGGAGGCCATAAATATATTCAG |
| | | AGTAACACAGGATTCACTGTTTTATTCTAGCAAT |
| | | GAATTTGAAGATTACCCTGGAAGAAGAAACATCT |
| | | ACAGAATTAGCATTGGAAGCTATCCTCCAAGCAA |
| | | GAAGTGTGTTACTTGCCATCTAAGGAAAGAAAGG |
| | | TGCCAATATTACACAGCAAGTTTCAGCGACTACG |
| | | CCAAGTACTATGCACTTGTCTGCTATGGCCCAGG |
| | | CATCCCCATTTCCACCCTTCATGACGGACGCACT |
| | | GATCAAGAAATTAAAATCCTGGAAGAAAACAAG |
| | | GAATTGGAAAATGCTTTGAAAAATATCCAGCTGC |
| | | CTAAAGAGGAAATTAAGAAACTTGAAGTAGATG |
| | | AAATTACTTTATGGTACAAGATGATTCTTCCTCCT |
| | | CAATTTGACAGATCAAAGAAGTATCCCTTGCTAA |
| | | TTCAAGTGTATGGTGGTCCCTGCAGTCAGAGTGT |
| | | AAGGTCTGTATTTGCTGTTAATTGGATATCTTATC |
| | | TTGCAAGTAAGGAAGGGATGGTCATTGCCTTGGT |
| | | GGATGGTCGGGGAACAGCTTTCCAAGGTGACAA |
| | | ACTCCTGTATGCAGTGTATCGAAAGCTGGGTGTT |
| | | TATGAAGTTGAAGACCAGATTACAGCTGTCAGAA |
| | | AATTCATAGAAATGGGTTTCATTGATGAAAAAAG |
| | | AATAGCCATATGGGGCTGGTCCTATGGAGGATAT |
| | | GTTTCATCACTGGCCCTTGCATCTGGAACTGGTCT |
| | | TTTCAAATGTGGGATAGCAGTGGCTCCAGTCTCC |
| | | AGCTGGGAATATTACGCGTCTGTCTACACAGAGA |
| | | GATTCATGGGTCTCCCAACAAAGGATGATAATCT |
| | | TGAGCACTATAAGAATTCAACTGTGATGGCAAGA |
| | | GCAGAATATTTCAGAAATGTAGACTATCTTCTCA |
| | | TCCACGGAACAGCAGATGATAATGTGCACTTTCA |
| | | AAACTCAGCACAGATTGCTAAAGCTCTGGTTAAT |
| | | GCACAAGTGGATTTCCAGGCAATGTGGTACTCTG |
| | | ACCAGAACCACGGCTTATCCGGCCTGTCCACGAA |
| | | CCACTTATACACCCACATGACCCACTTCCTAAAG |
| | | CAGTGTTTCTCTTTGTCAGACGGCAAAAAGAAAA |
| | | AGAAAAAGGGCCACCACCATCACCATCAC |
| 38 | human CEA | UniProt no. P06731 |
| 39 | human MCSP | UniProt no. Q6UVK1 |
| 40 | human EGFR | UniProt no. P00533 |
| 41 | human CD19 | UniProt no. P15391 |
| 42 | human CD20 | Uniprot no. P11836 |
| 43 | human CD33 | UniProt no. P20138 |
| 44 | human OX40L | UniProt no. P23510 |
| 45 | human CD27L | UniProt no. P32970 |
| 46 | human CD30L | UniProt no. P32971 |
| 47 | human 4-1BBL | UniProt no. P41273 |
| 48 | human LIGHT | UniProt no. O43557 |
| 49 | human GITRL | UniProt no. Q9UNG2 |
| 50 | hu 4-1BBL (50-254) | ACPWAVSGARASPGSAASPRLREGPELSPDDPAGL |
| | | LDLRQGMFAQLVAQNVLLIDGPLSWYSDPGLAGVS |
| | | LTGGLSYKEDTKELVVAKAGVYYVFFQLELRRVV |
| | | AGEGSGSVSLALHLQPLRSAAGAAALALTVDLPPA |
| | | SSEARNSAFGFQGRLLHLSAGQRLGVHLHTEARAR |
| | | HAWQLTQGATVLGLFRVTPEIPAGLPSPRSE |
| 51 | Petpide linker G4S | GGGGS |
| 52 | Peptide linker (G4S)₂ | GGGGSGGGGS |
| 53 | Peptide linker (SG4)₂ | SGGGGSGGGG |
| 54 | Peptide linker (G₄S)₃ | GGGGSGGGGSGGGGS |
| 55 | Peptide linker G4(SG4)₂ | GGGGSGGGGSGGGG |
| 56 | Peptide linker (G₄S)₄ | GGGGSGGGGSGGGGSGGGGS |
| 57 | Peptide linker | GSPGSSSSGS |
| 58 | Peptide linker | GSGSGSGS |
| 59 | Peptide linker | GSGSGNGS |
| 60 | Peptide linker | GGSGSGSG |
| 61 | Peptide linker | GGSGSG |
| 62 | Peptide linker | GGSG |
| 63 | Peptide linker | GGSGNGSG |
| 64 | Peptide linker | GGNGSGSG |
| 65 | Peptide linker | GGNGSG |
| 66 | human TRAF2 | UniProt no. Q12933 |
| 67 | human Thrombospondin 1 | UniProt no. P07996 |
| 68 | human Matrilin-4 | UniProt no. O95460 |
| 69 | human Cubilin | UniProt no. O60494 |
| 70 | isoleucine zipper domain | IKQIEDKIEE ILSKIYHIEN EIARIKKLIG ER |
| 71 | FAP(28H1)(VHCH1)-CMPtd-4- | see Table 1 |
| | 1BBL | |
| 72 | FAP(28H1)(VHCH1)- | see Table 8 |
| | CMPtd-Ox40L(52-183) | |
| 73 | Nucleotide sequence of | see Table 1 |
| | FAP(28H1)(VHCH1)-CMPtd-4- | |
| | 1BBL | |
| 74 | Nucleotide sequence of | see Table 8 |
| | FAP(28H1)(VHCH1)- | |
| | CMPtd-Ox40L(52-183) | |
| 75 | Nucleotide sequence of anti- | see Table 1 |
| | FAP(28H1) light chain | |
| 76 | anti-FAP(28H1) light chain | see Table 1 |
| 77 | Nucleotide sequence of DP47 | see Table 4 |
| | (VHCH1)-CMPtd-4- | |
| | 1BBL(71-254) | |
| 78 | Nucleotide sequence of | see Table 4 |
| | Germline control (DP47) light | |
| | chain | |
| 79 | DP47(VHCH1)-CMPtd-4- | see Table 4 |
| | 1BBL(71-254) | |
| 80 | Germline control (DP47) light | see Table 4 |
| | chain | |
| 81 | Nucleotide sequence of DP47 | see Table 6 |
| | heavy chain (hu IgG1 PG | |
| | LALA) | |
| 82 | DP47 heavy chain (hu IgG1 | see Table 6 |
| | PG LALA) | |
| 83 | Human 4-1BB ECD | Uniprot No. Q07011, aa 24-186 |
| 84 | Cynomolgus 4-1BB ECD | aa 24-186 |
| 85 | Murine 4-1BB ECD | Uniprot No. P20334, aa 24-187 |
| 86 | Nucleotide sequence of Fc | see Table 11 |
| | hole chain | |
| 87 | Nucleotide sequence of | see Table 11 |
| | human 4-1BB antigen Fc | |
| | knob chain | |
| 88 | Nucleotide sequence of | see Table 11 |
| | cynomolgus 4-1BB antigen | |
| | Fc knob chain | |
| 89 | Nucleotide sequence of | see Table 11 |
| | murine 4-1BB antigen Fc | |
| | knob chain | |
| 90 | Fc hole chain | see Table 11 |
| 91 | human 4-1BB antigen Fc | see Table 11 |
| | knob chain | |
| 92 | cynomolgus 4-1BB antigen | see Table 11 |
| | Fc knob chain | |
| 93 | murine 4-1BB antigen Fc | see Table 11 |
| | knob chain | |

Aspects of the invention are the following:
A trimeric costimulatory TNF family ligand-containing antigen binding molecule comprising three fusion polypeptides, each of the three fusion polypeptides comprising
   (a) an ectodomain of a co stimulatory TNF ligand family member or fragments thereof,
   (b) a trimerization domain, and
   (c) a moiety capable of specific binding to a target cell antigen
The trimeric costimulatory TNF family ligand-containing antigen binding molecule as defined in the first point above comprising three fusion polypeptides, each of the three fusion polypeptides comprising
   (a) an ectodomain of a co stimulatory TNF ligand family member or fragments thereof,
   (b) a trimerization domain derived from human cartilage matrix protein (huCMP) of amino acid sequence of SEQ ID NO:1, and
   (c) a moiety capable of specific binding to a target cell antigen.
The trimeric costimulatory TNF family ligand-containing antigen binding molecule as defined in the first and second points above, wherein the trimerization domain comprises an amino acid sequence having at least 95% identity to SEQ ID NO:2.
The trimeric costimulatory TNF family ligand-containing antigen binding molecule as defined in the first and second points above, wherein the trimerization domain comprises the amino acid sequence of SEQ ID NO:2.
The trimeric costimulatory TNF family ligand-containing antigen binding molecule as defined herein before, wherein the costimulatory TNF ligand family member is selected from 4-1BBL and OX40L.
The trimeric costimulatory TNF family ligand-containing antigen binding molecule as defined herein before, wherein the costimulatory TNF ligand family member is 4-1BBL.
The trimeric costimulatory TNF family ligand-containing antigen binding molecule as defined herein before, wherein the ectodomain of a TNF ligand family member comprises the amino acid sequence selected from the group consisting of SEQ ID NO:3, SEQ ID NO:4, SEQ ID NO:5, SEQ ID NO:6, SEQ ID NO:7, SEQ ID NO:8, SEQ ID NO:9 and SEQ ID NO:10, particularly the amino acid sequence of SEQ ID NO:3 or SEQ ID NO:7.
The trimeric costimulatory TNF family ligand-containing antigen binding molecule as defined herein before, comprising three fusion polypeptides, each of the three fusion polypeptides comprising
   (a) an ectodomain of a TNF ligand family comprising the amino acid sequence selected from the group consisting of SEQ ID NO:3, SEQ ID NO:4, SEQ ID NO:5, SEQ ID NO:6, SEQ ID NO:7, SEQ ID NO:8, SEQ ID NO:9 and SEQ ID NO:10,
   (b) a trimerization domain comprising the amino acid sequence of SEQ ID NO:2 and
   (c) a moiety capable of specific binding to a target cell antigen.
The trimeric costimulatory TNF family ligand-containing antigen binding molecule as defined herein before, wherein the costimulatory TNF ligand family member is OX40L.
The trimeric costimulatory TNF family ligand-containing antigen binding molecule as defined herein before, wherein the ectodomain of a TNF ligand family member comprises the amino acid sequence of SEQ ID NO:11 or SEQ ID NO:12.
The trimeric costimulatory TNF family ligand-containing antigen binding molecule as defined herein before, comprising three fusion polypeptides, wherein each of the three fusion polypeptides comprises
   (a) an ectodomain of a TNF ligand family comprising the amino acid sequence of SEQ ID NO:11 or SEQ ID NO:12,
   (b) a trimerization domain comprising the amino acid sequence of SEQ ID NO:2 and
   (c) a moiety capable of specific binding to a target cell antigen.
The trimeric costimulatory TNF family ligand-containing antigen binding molecule as defined herein before, wherein the ectodomain of a TNF ligand family member or a fragment thereof is fused at the N-terminal amino acid to the C-terminal amino acid of the trimerization domain, optionally through a peptide linker.
The trimeric costimulatory TNF family ligand-containing antigen binding molecule as defined herein before, wherein the moiety capable of specific binding to a target cell antigen is fused at the C-terminal amino acid to the N-terminal amino acid of the trimerization domain, optionally through a peptide linker.
The trimeric costimulatory TNF family ligand-containing antigen binding molecule as defined herein before, wherein the moiety capable of specific binding to a target cell antigen is selected from the group consisting of an antibody fragment, a Fab molecule, a crossover Fab molecule, a single chain Fab molecule, a Fv molecule, a scFv molecule, a single domain antibody, an aVH and a scaffold antigen binding protein.
The trimeric costimulatory TNF family ligand-containing antigen binding molecule as defined herein before, wherein the moiety capable of specific binding to a target cell antigen is a Fab molecule capable of specific binding to a target cell antigen.
The trimeric costimulatory TNF family ligand-containing antigen binding molecule as defined herein before, wherein said Fab molecule is fused at the C-terminal amino acid of the CH1 domain to the N-terminal amino acid of the trimerization domain, optionally through a peptide linker.
The trimeric costimulatory TNF family ligand-containing antigen binding molecule as defined herein before, wherein the target cell antigen is selected from the group consisting of Fibroblast Activation Protein (FAP), Melanoma-associated Chondroitin Sulfate Proteoglycan (MCSP), Epidermal Growth Factor Receptor (EGFR), Carcinoembryonic Antigen (CEA), CD19, CD20 and CD33.
The trimeric costimulatory TNF family ligand-containing antigen binding molecule as defined herein before, wherein the target cell antigen is Fibroblast Activation Protein (FAP).
The trimeric costimulatory TNF family ligand-containing antigen binding molecule as defined herein before, wherein the moiety capable of specific binding to FAP comprises
   (a) a VH domain comprising (i) CDR-H1 comprising the amino acid sequence of SEQ ID NO:13, (ii) CDR-H2 comprising the amino acid sequence of SEQ ID NO:14 and (iii) CDR-H3 comprising the amino acid sequence of SEQ ID NO:15, and a VL domain comprising (iv) CDR-L1 comprising the amino acid sequence of SEQ ID NO:16, (v) CDR-L2 comprising the amino acid sequence of SEQ ID NO:17 and (vi) CDR-L3 comprising the amino acid sequence of SEQ ID NO:18 or
   (b) a VH domain comprising (i) CDR-H1 comprising the amino acid sequence of SEQ ID NO:19, (ii) CDR-H2 comprising the amino acid sequence of SEQ ID NO:20 and (iii) CDR-H3 comprising the amino acid sequence of SEQ ID NO:21, and a VL domain comprising (iv) CDR-L1 comprising the amino acid sequence of SEQ ID NO:22, (v) CDR-L2 comprising the amino acid sequence of SEQ ID NO:23 and (vi) CDR-L3 comprising the amino acid sequence of SEQ ID NO:24.
The trimeric costimulatory TNF family ligand-containing antigen binding molecule as defined herein before, wherein said three fusion polypeptides are identical.
A fusion polypeptide comprising (a) an ectodomain of a costimulatory TNF ligand family member or a fragment thereof and (b) a trimerization domain derived from human cartilage matrix protein (huCMP) comprising the amino acid sequence of SEQ ID NO:2, wherein said trimerization domain is capable of mediating stable association of said fusion polypeptide with two further such fusion polypeptides.
The fusion polypeptide as defined herein before, wherein the fusion polypeptide further comprises (c) a moiety capable of specific binding to a target cell antigen.
The fusion polypeptide as defined herein before, wherein the fusion polypeptide comprises
   (a) an ectodomain of a TNF ligand family comprising the amino acid sequence selected from the group consisting of SEQ ID NO:3, SEQ ID NO:4, SEQ ID NO:5, SEQ ID NO:6, SEQ ID NO:7, SEQ ID NO:8, SEQ ID NO:9 and SEQ ID NO:10,
   (b) a trimerization domain comprising the amino acid sequence of SEQ ID NO:2 and
   (c) a VH domain comprising (i) CDR-H1 comprising the amino acid sequence of SEQ ID NO:13, (ii) CDR-H2 comprising the amino acid sequence of SEQ ID NO:14 and (iii) CDR-H3 comprising the amino acid sequence of SEQ ID NO:15 or a VH domain comprising (i) CDR-H1 comprising the amino acid sequence of SEQ ID NO:19, (ii) CDR-H2 comprising the amino acid sequence of SEQ ID NO:20 and (iii) CDR-H3 comprising the amino acid sequence of SEQ ID NO:21.
A polynucleotide encoding the trimeric costimulatory TNF family ligand-containing antigen binding molecule as defined herein before or the fusion polypeptide as defined herein before.
An expression vector comprising the polynucleotide as defined herein before.
A host cell comprising the polynucleotide as defined herein before or the expression vector as defined herein before.
A method of producing a trimeric costimulatory TNF family ligand-containing antigen binding molecule, comprising culturing the host cell as defined herein before under conditions suitable for the expression of said trimeric antigen binding molecule and isolating said trimeric antigen binding molecule.
A trimeric costimulatory TNF family ligand-containing antigen binding molecule produced by the method as defined herein before.
A pharmaceutical composition comprising the trimeric costimulatory TNF family ligand-containing antigen binding molecule as defined herein before and at least one pharmaceutically acceptable excipient.
The trimeric costimulatory TNF family ligand-containing antigen binding molecule as defined herein before, or the pharmaceutical composition of claim 29, for use as a medicament.
The trimeric costimulatory TNF family ligand-containing antigen binding molecule as defined herein before, or the pharmaceutical composition as defined herein before, for use in the treatment of cancer.
The trimeric costimulatory TNF family ligand-containing antigen binding molecule as defined herein before, or the pharmaceutical composition as defined herein before, for use in up-regulating or prolonging cytotoxic T cell activity.
Use of the trimeric costimulatory TNF family ligand-containing antigen binding molecule as defined herein before, or the pharmaceutical composition as defined herein before, in the manufacture of a medicament for the treatment of cancer.
Use of the trimeric costimulatory TNF family ligand-containing antigen binding molecule as defined herein before, or the pharmaceutical composition as defined herein before, in the manufacture of a medicament for up-regulating or prolonging cytotoxic T cell activity.
A method of treating an individual having cancer comprising administering to the individual an effective amount of the trimeric costimulatory TNF family ligand-containing antigen binding molecule as defined herein before, or the pharmaceutical composition as defined herein before.
A method of up-regulating or prolonging cytotoxic T cell activity in an individual having cancer comprising administering to the individual an effective amount of the trimeric costimulatory TNF family ligand-containing antigen binding molecule as defined herein before, or the pharmaceutical composition as defined herein before.

### EXAMPLES

The following are examples of methods and compositions of the invention. It is understood that various other embodiments may be practiced, given the general description provided above.

Recombinant DNA techniques

Standard methods were used to manipulate DNA as described in Sambrook et al., Molecular cloning: A laboratory manual; Cold Spring Harbor Laboratory Press, Cold Spring Harbor, N.Y., 1989. The molecular biological reagents were used according to the manufacturer's instructions. General information regarding the nucleotide sequences of human immunoglobulin light and heavy chains is given in: Kabat, E. A. et al., (1991) Sequences of Proteins of Immunological Interest, Fifth Ed., NIH Publication No 91-3242.

### DNA Sequencing

DNA sequences were determined by double strand sequencing.

### Gene Synthesis

Desired gene segments were either generated by PCR using appropriate templates or were synthesized by Geneart AG (Regensburg, Germany) from synthetic oligonucleotides and PCR products by automated gene synthesis. In cases where no exact gene sequence was available, oligonucleotide primers were designed based on sequences from closest homologues and the genes were isolated by RT-PCR from RNA originating from the appropriate tissue. The gene segments flanked by singular restriction endonuclease cleavage sites were cloned into standard cloning/sequencing vectors. The plasmid DNA was purified from transformed bacteria and concentration determined by UV spectroscopy. The DNA sequence of the subcloned gene fragments was confirmed by DNA sequencing. Gene segments were designed with suitable restriction sites to allow sub-cloning into the respective expression vectors. All constructs were designed with a 5′-end DNA sequence coding for a leader peptide which targets proteins for secretion in eukaryotic cells.

### Example 1

### 1.1. Preparation and Purification of Targeted huCMP Trimeric 4-1BB Ligand-Containing Antigen Binding Molecules

To enhance binding of Fab molecules to specific members of the TNF receptor superfamily and thereby enhanced cross-linking of these receptors, trimerized Fab molecules targeting 4-1BB were generated.

The Fab genes (VH-CH1) against fibroblast activation protein (FAP clone 28H1) were fused via a (G4S)₂ linker to a short trimerization domain derived from human cartilage matrix protein (huCMP) (Uniprot Accession: P21941, SEQ ID NO:1; Residues 454 to 496, SEQ ID NO.:2) by standard recombinant DNA technologies. The cysteine residues forming interchain disulfide bridges at positions 458 and 460 were used together with the coiled coil domain comprising residues 467 to 495.

The DNA sequence encoding part of the ectodomain (amino acids 71-254 or 71-248) of human 4-1BB ligand was synthetized according to P41273 (Uniprot Accession No:) and fused via a (G4S)₄ linker downstream of the trimerization domain.

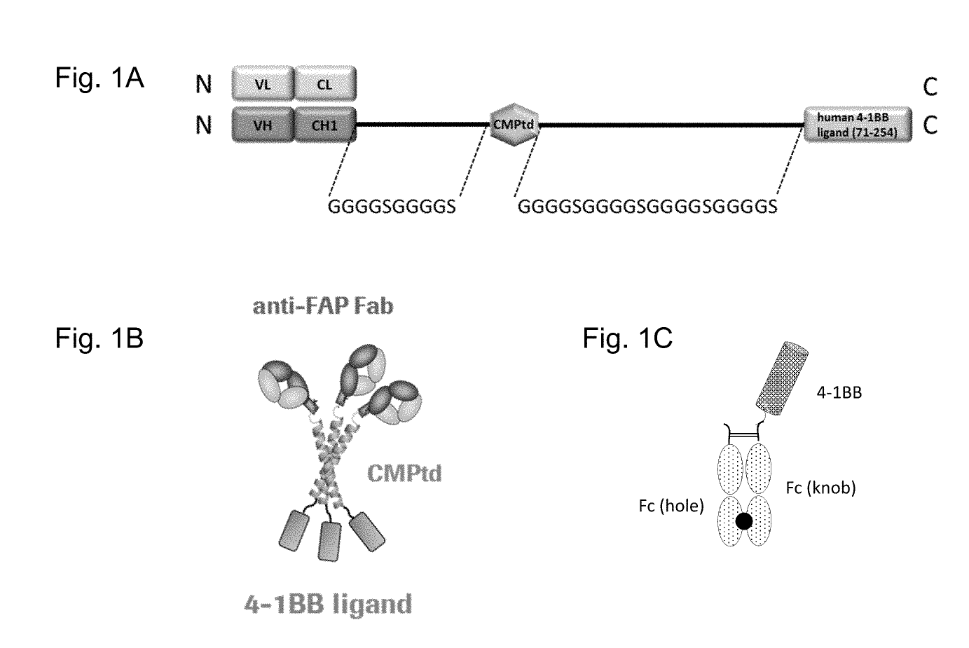

The chain encoding FAP-targeted 4-1BB ligand and the light chain with FAP specificity are shown in FIG. 1a. Disulphide bonds are formed between three FAP-targeted 4-1BB ligand chains leading to the formation of covalently linked CMP-trimeric 4-1BB ligand antigen binding molecules, targeted to FAP. FIG. 1b shows the trimeric form.

Table 1 shows, respectively, the cDNA and amino acid sequences of the trimeric FAP-targeted 4-1BBL-containing fusion polypeptide, which is trimerizing via CMP trimerization domain (CMPtd) to form a trimeric FAP-targeted 4-1BBL-containing antigen binding molecule of the invention.

TABLE 1
cDNA and amino acid sequences of FAP(28H1)-targeted
hu CMP (CMPtd) trimeric human 4-1BBL(71-254)-containing
antigen binding molecule

| SEQ ID | | |
|---|---|---|
| NO: | Description | Sequence |
| 73 | nucleotide sequence of | GAAGTGCAGCTGCTGGAATCCGGCGGAGGCCTG |
| | FAP(28H1)(VHCH1)- | GTGCAGCCTGGCGGATCTCTGAGACTGTCCTGC |
| | CMPtd-4-1BBL (71-254) | GCCGCCTCCGGCTTCACCTTCTCCTCCCACGCCA |
| | | TGTCCTGGGTCCGACAGGCTCCTGGCAAAGGCC |
| | | TGGAATGGGTGTCCGCCATCTGGGCCTCCGGCG |
| | | AGCAGTACTACGCCGACTCTGTGAAGGGCCGGT |
| | | TCACCATCTCCCGGGACAACTCCAAGAACACCC |
| | | TGTACCTGCAGATGAACTCCCTGCGGGCCGAGG |
| | | ACACCGCCGTGTACTACTGTGCCAAGGGCTGGC |
| | | TGGGCAACTTCGACTACTGGGGACAGGGCACCC |
| | | TGGTCACCGTGTCCAGCGCTAGCACAAAGGGAC |
| | | CTAGCGTGTTCCCCCTGGCCCCCAGCAGCAAGT |
| | | CTACATCTGGCGGAACAGCCGCCCTGGGCTGCC |
| | | TCGTGAAGGACTACTTTCCCGAGCCCGTGACCG |
| | | TGTCCTGGAACTCTGGCGCTCTGACAAGCGGCG |
| | | TGCACACCTTTCCAGCCGTGCTGCAGAGCAGCG |
| | | GCCTGTACTCTCTGAGCAGCGTCGTGACAGTGC |
| | | CCAGCAGCTCTCTGGGCACCCAGACCTACATCT |
| | | GCAACGTGAACCACAAGCCCAGCAACACCAAG |
| | | GTGGACAAGAAGGTGGAACCCAAGAGCTGCGA |
| | | CGGCGGAGGGGGATCTGGCGGCGGAGGATCCG |
| | | AGGAAGATCCTTGCGCCTGCGAGAGCCTCGTGA |
| | | AGTTCCAGGCCAAGGTGGAAGGACTGCTGCAG |
| | | GCCCTGACCCGGAAACTGGAAGCCGTGTCCAAG |
| | | CGGCTGGCCATCCTGGAAAACACCGTGGTGTCC |
| | | GGAGGCGGAGGATCTGGCGGCGGAGGAAGTGG |
| | | CGGAGGCGGATCTGGCGGCGGAGGATCTAGAG |
| | | AGGGACCCGAACTGTCCCCTGACGATCCAGCCG |
| | | GGCTGCTGGATCTGAGACAGGGAATGTTCGCCC |
| | | AGCTGGTGGCTCAGAATGTGCTGCTGATTGACG |
| | | GACCTCTGAGCTGGTACTCCGACCCAGGGCTGG |
| | | CAGGGGTGTCCCTGACTGGGGGACTGTCCTACA |
| | | AAGAAGATACAAAAGAACTGGTGGTGGCTAAA |
| | | GCTGGGGTGTACTATGTGTTTTTTCAGCTGGAAC |
| | | TGAGGCGGGTGGTGGCTGGGGAGGGCTCAGGA |
| | | TCTGTGTCCCTGGCTCTGCATCTGCAGCCACTGC |
| | | GCTCTGCTGCTGGCGCAGCTGCACTGGCTCTGA |
| | | CTGTGGACCTGCCACCAGCCTCTAGCGAGGCCA |
| | | GAAACAGCGCCTTCGGGTTCCAAGGACGCCTGC |
| | | TGCATCTGAGCGCCGGACAGCGCCTGGGAGTGC |
| | | ATCTGCATACTGAAGCCAGAGCCCGGCATGCTT |
| | | GGCAGCTGACTCAGGGGGCAACTGTGCTGGGAC |
| | | TGTTTCGCGTGACACCTGAGATCCCTGCCGGAC |
| | | TGCCAAGCCCTAGATCAGAA |
| 75 | nucleotide sequence of | GAGATCGTGCTGACCCAGTCCCCCGGCACCCTG |
| | anti-FAP(28H1) light | TCTCTGAGCCCTGGCGAGAGAGCCACCCTGTCC |
| | chain | TGCAGAGCCTCCCAGTCCGTGTCCCGGTCCTAC |
| | | CTCGCCTGGTATCAGCAGAAGCCCGGCCAGGCC |
| | | CCTCGGCTGCTGATCATCGGCGCCTCTACCAGA |
| | | GCCACCGGCATCCCTGACCGGTTCTCCGGCTCT |
| | | GGCTCCGGCACCGACTTCACCCTGACCATCTCC |
| | | CGGCTGGAACCCGAGGACTTCGCCGTGTACTAC |
| | | TGCCAGCAGGGCCAGGTCATCCCTCCCACCTTT |
| | | GGCCAGGGCACCAAGGTGGAAATCAAGCGTAC |
| | | GGTGGCTGCACCATCTGTCTTCATCTTCCCGCCA |
| | | TCTGATGAGCAGTTGAAATCTGGAACTGCCTCT |
| | | GTTGTGTGCCTGCTGAATAACTTCTATCCCAGA |
| | | GAGGCCAAAGTACAGTGGAAGGTGGATAACGC |
| | | CCTCCAATCGGGTAACTCCCAGGAGAGTGTCAC |
| | | AGAGCAGGACAGCAAGGACAGCACCTACAGCC |
| | | TCAGCAGCACCCTGACGCTGAGCAAAGCAGACT |
| | | ACGAGAAACACAAAGTCTACGCCTGCGAAGTC |
| | | ACCCATCAGGGCCTGAGCTCGCCCGTCACAAAG |
| | | AGCTTCAACAGGGGAGAGTGT |
| 71 | FAP(28H1)(VHCH1)- | EVQLLESGGGLVQPGGSLRLSCAASGFTFSSHAMS |
| | CMPtd-4-1BBL(71-254) | WVRQAPGKGLEWVSAIWASGEQYYADSVKGRFT |
| | | ISRDNSKNTLYLQMNSLRAEDTAVYYCAKGWLG |
| | | NFDYWGQGTLVTVSSASTKGPSVFPLAPSSKSTSG |
| | | GTAALGCLVKDYFPEPVTVSWNSGALTSGVHTFP |
| | | AVLQSSGLYSLSSVVTVPSSSLGTQTYICNVNHKP |
| | | SNTKVDKKVEPKSCDGGGGSGGGGSEEDPCACES |
| | | LVKFQAKVEGLLQALTRKLEAVSKRLAILENTVV |
| | | SGGGGSGGGGSGGGGSGGGGSREGPELSPDDPAG |
| | | LLDLRQGMFAQLVAQNVLLIDGPLSWYSDPGLAG |
| | | VSLTGGLSYKEDTKELVVAKAGVYYVFFQLELRR |
| | | VVAGEGSGSVSLALHLQPLRSAAGAAALALTVDL |
| | | PPASSEARNSAFGFQGRLLHLSAGQRLGVHLHTE |
| | | ARARHAWQLTQGATVLGLFRVTPEIPAGLPSPRSE |
| 76 | Anti-FAP(28H1) light | EIVLTQSPGTLSLSPGERATLSCRASQSVSRSYLAW |
| | chain | YQQKPGQAPRLLIIGASTRATGIPDRFSGSGSGTDF |
| | | TLTISRLEPEDFAVYYCQQGQVIPPTFGQGTKVEIK |
| | | RTVAAPSVFIFPPSDEQLKSGTASVVCLLNNFYPRE |
| | | AKVQWKVDNALQSGNSQESVTEQDSKDSTYSLSS |
| | | TLTLSKADYEKHKVYACEVTHQGLSSPVTKSFNR |
| | | GEC |

The sequences encoding the trimeric FAP-targeted 4-1BBL-containing antigen binding molecule were cloned into a plasmid vector, which drives expression of the insert from an MPSV promoter and contains a synthetic polyA signal sequence located at the 3′ end of the CDS. In addition, the vector contains an EBV OriP sequence for episomal maintenance of the plasmid.

The trimeric FAP-targeted 4-1BBL-containing antigen binding molecule was produced by co-transfecting HEK293-EBNA cells with the mammalian expression vectors using polyethylenimine. The cells were transfected with the corresponding expression vectors in a 1:1 ratio (“vector FAP(VHCH1)-CMPtd-4-1BBL”: “vector light chain”).

For production in 500 mL shake flasks, 400 million HEK293 EBNA cells were seeded 24 hours before transfection. For transfection cells were centrifuged for 5 minutes by 210×g, and supernatant was replaced by pre-warmed CD CHO medium. Expression vectors were mixed in 20 mL CD CHO medium to a final amount of 200 μg DNA. After addition of 540 μL PEI, the solution was vortexed for 15 seconds and incubated for 10 minutes at room temperature. Afterwards, cells were mixed with the DNA/PEI solution, transferred to a 500 mL shake flask and incubated for 3 hours at 37° C. in an incubator with a 5% CO₂ atmosphere. After the incubation, 160 mL F17 medium was added and cells were cultured for 24 hours. One day after transfection 1 mM valproic acid and 7% Feed with supplements were added. After culturing for 7 days, the cell supernatant was collected by centrifugation for 15 minutes at 210×g. The solution was sterile filtered (0.22 μm filter), supplemented with sodium azide to a final concentration of 0.01% (w/v), and kept at 4° C.

The trimeric FAP-targeted huCMP trimeric 4-1BBL-containing antigen binding molecule was purified from cell culture supernatants by affinity chromatography via CH1 domain of human IgG antibodies, followed by size exclusion chromatography. For affinity chromatography, the supernatant was loaded on a column packed with CaptureSelect IgG-CH1 matrix (Column Volume=1 ml; BAC, The Netherlands) and equilibrated with 5 ml 50 mM Tris(hydroxymethyl)-aminomethan (TRIS), 100 mM Glycine, 150 mM sodium chloride, pH 8.0. Unbound protein was removed by washing with at least 10 column volumes of the same buffer. The bound protein was eluted by applying a linear pH-gradient over 20 column volumes from 50 mM TRIS, 100 mM Glycine, 150 mM sodium chloride pH 8.0 to pH 2.0. The column was then washed with 10 column volumes of 50 mM TRIS, 100 mM Glycine, 150 mM sodium chloride, pH 2.0.

The pH of collected fractions was neutralized by adding 1/40 (v/v) of 2M Tris, pH8.0. The protein was concentrated prior to loading on a HiLoad Superdex 200 column (GE Healthcare) equilibrated with 20 mM Histidine, 140 mM sodium chloride, 0.01% (v/v) Tween/20 solution of pH 6.0.

The protein concentration was determined by measuring the optical density (OD) at 280 nm, using the molar extinction coefficient calculated on the basis of the amino acid sequence. Purity and molecular weight of the trimeric FAP-targeted 4-1BBL-containing antigen binding molecule was analyzed by SDS-PAGE in the presence and absence of a reducing agent (5 mM 1,4-dithiotreitol) and staining with Coomassie SimpleBlue™ SafeStain (Invitrogen USA). The aggregate content of samples was analyzed using a TSKgel G3000 SW XL analytical size-exclusion column (Tosoh) equilibrated in 25 mM K₂HPO₄, 125 mM NaCl, 200 mM L-Arginine Monohydrocloride, 0.02% (w/v) NaN3, pH 6.7 running buffer at 25° C.

The molecular weight of the huCMP containing construct was determined under non-reducing and reducing conditions by LC-MS using a Agilent HPLC 1200 coupled to a TOF 6441 mass spectrometer (Agilent). For analyses under reduced conditions sample was incubated for 30 minutes at 37° C. in 10 μl 8 M Guanidine-HCl and 10 μl 0.5 mM TCEP diluted in 4 M Guanidine-HCl. Non-reduced samples were directly used for LC-MS analyses. The chromatographic separation was performed on a Macherey & Nagel Polysterene column; RP1000-8 (8 μm particle size, 4.6×250 mm; cat. No. 719510) using the program shown in Table 2. Eluent A was 5% acetonitrile and 0.05% (v/v) formic acid in water, eluent B was 95% acetonitrile, 5% water and 0.05% formic acid. The separation was performed at 40° C. with a flow of 1 ml/min and 7 μg (15 μl) of the sample was injected.

During the first 4 minutes the eluate is directed into the waste to prevent the mass spectrometer from salt contamination. The ESI-source was running with a drying gas flow of 12 l/min, a temperature of 350° C. and a nebulizer pressure of 60 psi. The MS spectra are acquired using a fragmentor voltage of 350 V and a mass range 700 to 3200 m/z in positive ion mode. MS data are acquired by the instrument software from 4 to 17 minutes.

TABLE 2
Mixture of solvents in HPLC chromatography for separation
of individual compounds for Mass Spectrometry analysis.

| Time (min.) | % solvent A | % solvent B |
|---|---|---|
| 0.5 | 85 | 15 |
| 10 | 40 | 60 |
| 12.5 | 0 | 100 |
| 14.5 | 0 | 100 |
| 14.6 | 85 | 15 |
| 16 | 85 | 15 |
| 16.1 | 0 | 100 |

Solvent A is: 5% acetonitrile and 0.05% (v/v) formic acid in water, and solvent B is: 95% acetonitrile, 5% water and 0.05% formic acid

All used analytical methods confirmed homogeneous preparation of the trimerized molecules. Table 3 summarizes yield, final monomer content and mass of the FAP-targeted trimeric 4-1BBL-containing antigen binding molecule.

TABLE 3
Biochemical analysis of targeted CMP-trimeric
4-1BBL-containing antigen binding molecule

| | Trimer | CE-SDS | CE-SDS | | | Thermal |
|---|---|---|---|---|---|---|
| | [%] | non red | red | LC/MS | LC/MS | stability |
| Construct | (SEC) | [%] | [%] | (non red) | (red) | [° C.] |
| trimeric | 100% | 92.2% | 31.1% | Theoretical: | (VL-CL) | 51° C. |
| FAP(28H1)- | (439.2 kDa) | (218 kDa) | (27.4 kDa) | 218375.2 Da | Theoretical: | |
| targeted 4- | | | 67.1% | Experimental: | 23367.13 Da | |
| 1BBL(71-254)- | | | (55.7 kDa) | 218404.2 Da | Experimental: | |
| containing | | | | (98.1%) | 23371.8 Da | |
| antigen binding | | | | | (VH-CH1- | |
| molecule | | | | | huCMP-flag) | |
| | | | | | Theoretical: | |
| | | | | | 49424.61 Da | |
| | | | | | Experimental: | |
| | | | | | 49431.7 Da | |

Trimer content of the purified molecules was determined by size exclusion chromatography (SEC) and liquid chromatography mass spectrometry (LC/MS). Thermal stability of the molecules was analyzed by Dynamic Light Scattering (DLS) experiment. In brief, 30 μg of filtered protein sample with a protein concentration of 1 mg/ml is applied in duplicate to a Dynapro plate reader (Wyatt Technology Corporation; USA). The temperature is ramped from 25 to75° C. at 0.05° C./min, with the radius and total scattering intensity being collected.

### 1.2. Preparation and Purification of Untargeted huCMP Trimeric 4-1BB Ligand-Containing Antigen Binding Molecules

The Fab genes (VH-CH1) from a germline control (DP47) were fused via a (G4S)₂ linker to a short trimerization domain derived from human CMP (Uniprot Accession: P21941; Residues 454 to 496, SEQ ID NO:2) by standard recombinant DNA technologies, as described for the trimeric FAP-targeted 4-1BB ligand-containing antigen binding molecules.

Table 4 shows, respectively, the cDNA and amino acid sequences of the trimeric untargeted 4-1BBL(71-254)-containing antigen binding molecule, which is trimerizing via CMP trimerization domain.

TABLE 4
cDNA and amino acid sequences of DP47 untargeted
human CMP (CMPtd) trimeric 4-1BBL(71-254)-containing
antigen binding molecule

| SEQ ID | | |
|---|---|---|
| NO: | Description | Sequence |
| 77 | nucleotide sequence of | GAGGTGCAATTGTTGGAGTCTGGGGGAGGCTTG |
| | DP47 (VHCH1)-CMPtd- | GTACAGCCTGGGGGGTCCCTGAGACTCTCCTGT |
| | 4-1BBL(71-254) | GCAGCCTCCGGATTCACCTTTAGCAGTTATGCC |
| | | ATGAGCTGGGTCCGCCAGGCTCCAGGGAAGGG |
| | | GCTGGAGTGGGTCTCAGCTATTAGTGGTAGTGG |
| | | TGGTAGCACATACTACGCAGACTCCGTGAAGGG |
| | | CCGGTTCACCATCTCCAGAGACAATTCCAAGAA |
| | | CACGCTGTATCTGCAGATGAACAGCCTGAGAGC |
| | | CGAGGACACGGCCGTATATTACTGTGCGAAAGG |
| | | CAGCGGATTTGACTACTGGGGCCAAGGAACCCT |
| | | GGTCACCGTCTCGAGTGCTAGCACAAAGGGACC |
| | | TAGCGTGTTCCCCCTGGCCCCCAGCAGCAAGTC |
| | | TACATCTGGCGGAACAGCCGCCCTGGGCTGCCT |
| | | CGTGAAGGACTACTTTCCCGAGCCCGTGACCGT |
| | | GTCCTGGAACTCTGGCGCTCTGACAAGCGGCGT |
| | | GCACACCTTTCCAGCCGTGCTGCAGAGCAGCGG |
| | | CCTGTACTCTCTGAGCAGCGTCGTGACAGTGCC |
| | | CAGCAGCTCTCTGGGCACCCAGACCTACATCTG |
| | | CAACGTGAACCACAAGCCCAGCAACACCAAGG |
| | | TGGACAAGAAGGTGGAACCCAAGAGCTGCGAC |
| | | GGCGGAGGGGGATCTGGCGGCGGAGGATCCGA |
| | | GGAAGATCCTTGCGCCTGCGAGAGCCTCGTGAA |
| | | GTTCCAGGCCAAGGTGGAAGGACTGCTGCAGGC |
| | | CCTGACCCGGAAACTGGAAGCCGTGTCCAAGCG |
| | | GCTGGCCATCCTGGAAAACACCGTGGTGTCCGG |
| | | AGGCGGAGGATCTGGCGGCGGAGGAAGTGGCG |
| | | GAGGCGGATCTGGCGGCGGAGGATCTAGAGAG |
| | | GGACCCGAACTGTCCCCTGACGATCCAGCCGGG |
| | | CTGCTGGATCTGAGACAGGGAATGTTCGCCCAG |
| | | CTGGTGGCTCAGAATGTGCTGCTGATTGACGGA |
| | | CCTCTGAGCTGGTACTCCGACCCAGGGCTGGCA |
| | | GGGGTGTCCCTGACTGGGGGACTGTCCTACAAA |
| | | GAAGATACAAAAGAACTGGTGGTGGCTAAAGC |
| | | TGGGGTGTACTATGTGTTTTTTCAGCTGGAACTG |
| | | AGGCGGGTGGTGGCTGGGGAGGGCTCAGGATC |
| | | TGTGTCCCTGGCTCTGCATCTGCAGCCACTGCGC |
| | | TCTGCTGCTGGCGCAGCTGCACTGGCTCTGACT |
| | | GTGGACCTGCCACCAGCCTCTAGCGAGGCCAGA |
| | | AACAGCGCCTTCGGGTTCCAAGGACGCCTGCTG |
| | | CATCTGAGCGCCGGACAGCGCCTGGGAGTGCAT |
| | | CTGCATACTGAAGCCAGAGCCCGGCATGCTTGG |
| | | CAGCTGACTCAGGGGGCAACTGTGCTGGGACTG |
| | | TTTCGCGTGACACCTGAGATCCCTGCCGGACTG |
| | | CCAAGCCCTAGATCAGAA |
| 78 | nucleotide sequence of | GAAATCGTGTTAACGCAGTCTCCAGGCACCCTG |
| | Germline control (DP47) | TCTTTGTCTCCAGGGGAAAGAGCCACCCTCTCTT |
| | light chain | GCAGGGCCAGTCAGAGTGTTAGCAGCAGCTACT |
| | | TAGCCTGGTACCAGCAGAAACCTGGCCAGGCTC |
| | | CCAGGCTCCTCATCTATGGAGCATCCAGCAGGG |
| | | CCACTGGCATCCCAGACAGGTTCAGTGGCAGTG |
| | | GATCCGGGACAGACTTCACTCTCACCATCAGCA |
| | | GACTGGAGCCTGAAGATTTTGCAGTGTATTACT |
| | | GTCAGCAGTATGGTAGCTCACCGCTGACGTTCG |
| | | GCCAGGGGACCAAAGTGGAAATCAAACGTACG |
| | | GTGGCTGCACCATCTGTCTTCATCTTCCCGCCAT |
| | | CTGATGAGCAGTTGAAATCTGGAACTGCCTCTG |
| | | TTGTGTGCCTGCTGAATAACTTCTATCCCAGAG |
| | | AGGCCAAAGTACAGTGGAAGGTGGATAACGCC |
| | | CTCCAATCGGGTAACTCCCAGGAGAGTGTCACA |
| | | GAGCAGGACAGCAAGGACAGCACCTACAGCCT |
| | | CAGCAGCACCCTGACGCTGAGCAAAGCAGACT |
| | | ACGAGAAACACAAAGTCTACGCCTGCGAAGTC |
| | | ACCCATCAGGGCCTGAGCTCGCCCGTCACAAAG |
| | | AGCTTCAACAGGGGAGAGTGT |
| 79 | DP47(VHCH1)-CMPtd- | EVQLLESGGGLVQPGGSLRLSCAASGFTFSSYAMS |
| | 4-1BBL(71-254) | WVRQAPGKGLEWVSAISGSGGSTYYADSVKGRF |
| | | TISRDNSKNTLYLQMNSLRAEDTAVYYCAKGSGF |
| | | DYWGQGTLVTVSSASTKGPSVFPLAPSSKSTSGGT |
| | | AALGCLVKDYFPEPVTVSWNSGALTSGVHTFPAV |
| | | LQSSGLYSLSSVVTVPSSSLGTQTYICNVNHKPSNT |
| | | KVDKKVEPKSCDGGGGSGGGGSEEDPCACESLVK |
| | | FQAKVEGLLQALTRKLEAVSKRLAILENTVVSGG |
| | | GGSGGGGSGGGGSGGGGSREGPELSPDDPAGLLD |
| | | LRQGMFAQLVAQNVLLIDGPLSWYSDPGLAGVSL |
| | | TGGLSYKEDTKELVVAKAGVYYVFFQLELRRVV |
| | | AGEGSGSVSLALHLQPLRSAAGAAALALTVDLPP |
| | | ASSEARNSAFGFQGRLLHLSAGQRLGVHLHTEAR |
| | | ARHAWQLTQGATVLGLFRVTPEIPAGLPSPRSE |
| 80 | Germline control (DP47) | EIVLTQSPGTLSLSPGERATLSCRASQSVSSSYLAW |
| | light chain | YQQKPGQAPRLLIYGASSRATGIPDRFSGSGSGTD |
| | | FTLTISRLEPEDFAVYYCQQYGSSPLTFGQGTKVEI |
| | | KRTVAAPSVFIFPPSDEQLKSGTASVVCLLNNFYP |
| | | REAKVQWKVDNALQSGNSQESVTEQDSKDSTYS |
| | | LSSTLTLSKADYEKHKVYACEVTHQGLSSPVTKSF |
| | | NRGEC |

The sequences encoding the untargeted huCMP trimeric 4-1BB ligand-containing antigen binding molecule were cloned into a plasmid vector, which drives expression of the insert from an MPSV promoter and contains a synthetic polyA signal sequence located at the 3′ end of the CDS. In addition, the vector contains an EBV OriP sequence for episomal maintenance of the plasmid.

The untargeted huCMP trimeric 4-1BB ligand-containing antigen binding molecule was produced by co-transfecting HEK293-EBNA cells with the mammalian expression vectors using polyethylenimine. The cells were transfected with the corresponding expression vectors in a 1:1 ratio (“vector DP47(VHCH1)-CMPtd-4-1BBL”: “vector light chain”).

Production, purification and characterization of the untargeted CMP trimeric 4-1BB ligand-containing antigen binding molecule was performed as described above for the FAP-targeted CMP trimeric 4-1BB ligand-containing antigen binding molecule.

All used analytical methods confirmed homogeneous preparation of trimerized molecules. Table 5 summarizes the final monomer content of the untargeted CMP-trimeric 4-1BB ligand.

TABLE 5
Biochemical analysis of untargeted CMP-trimeric
4-1BBL-containing antigen binding molecule

| | Trimer [%] | CE-SDS non red | CE-SDS red |
|---|---|---|---|
| Construct | (SEC) | [%] | [%] |
| untargeted CMP- | 99.1% | 91.3% | 33.9% |
| trimeric 4-1BB | (468 kDa) | (217 kDa) | (27.5 kDa) |
| ligand-containing | | | 63.4% |
| antigen binding | | | (55.7 kDa) |
| molecule | | | |

### 1.3 Preparation of Untargeted Human IgG1 as Control F

An additional control molecule used in the assays was an untargeted DP47, germline control, human IgG1, containing the Pro329Gly, Leu234Ala and Leu235Ala mutations, to abrogate binding to Fc gamma receptors according to the method described in International Patent Appl. Publ. No. WO 2012/130831).

Table 6 shows the cDNA and amino acid sequences of the cDNA and amino acid sequences of the untargeted DP47 huIgG1 PGLALA (Control F).

TABLE 6
Sequences of untargeted DP47 huIgG1 (Control F)

| SEQ ID | | |
|---|---|---|
| NO: | Description | Sequence |
| 81 | nucleotide | GAGGTGCAATTGTTGGAGTCTGGGGGAGGCTTGGTACA |
| | sequence of DP47 | GCCTGGGGGGTCCCTGAGACTCTCCTGTGCAGCCTCCGG |
| | heavy chain (hu | ATTCACCTTTAGCAGTTATGCCATGAGCTGGGTCCGCCA |
| | IgG1 PGLALA) | GGCTCCAGGGAAGGGGCTGGAGTGGGTCTCAGCTATTA |
| | | GTGGTAGTGGTGGTAGCACATACTACGCAGACTCCGTGA |
| | | AGGGCCGGTTCACCATCTCCAGAGACAATTCCAAGAAC |
| | | ACGCTGTATCTGCAGATGAACAGCCTGAGAGCCGAGGA |
| | | CACGGCCGTATATTACTGTGCGAAAGGCAGCGGATTTGA |
| | | CTACTGGGGCCAAGGAACCCTGGTCACCGTCTCGAGTGC |
| | | TAGCACCAAGGGCCCATCGGTCTTCCCCCTGGCACCCTC |
| | | CTCCAAGAGCACCTCTGGGGGCACAGCGGCCCTGGGCT |
| | | GCCTGGTCAAGGACTACTTCCCCGAACCGGTGACGGTGT |
| | | CGTGGAACTCAGGCGCCCTGACCAGCGGCGTGCACACC |
| | | TTCCCGGCTGTCCTACAGTCCTCAGGACTCTACTCCCTC |
| | | AGCAGCGTGGTGACCGTGCCCTCCAGCAGCTTGGGCAC |
| | | CCAGACCTACATCTGCAACGTGAATCACAAGCCCAGCA |
| | | ACACCAAGGTGGACAAGAAAGTTGAGCCCAAATCTTGT |
| | | GACAAAACTCACACATGCCCACCGTGCCCAGCACCTGA |
| | | AGCTGCAGGGGGACCGTCAGTCTTCCTCTTCCCCCCAAA |
| | | ACCCAAGGACACCCTCATGATCTCCCGGACCCCTGAGGT |
| | | CACATGCGTGGTGGTGGACGTGAGCCACGAAGACCCTG |
| | | AGGTCAAGTTCAACTGGTACGTGGACGGCGTGGAGGTG |
| | | CATAATGCCAAGACAAAGCCGCGGGAGGAGCAGTACAA |
| | | CAGCACGTACCGTGTGGTCAGCGTCCTCACCGTCCTGCA |
| | | CCAGGACTGGCTGAATGGCAAGGAGTACAAGTGCAAGG |
| | | TCTCCAACAAAGCCCTCGGCGCCCCCATCGAGAAAACC |
| | | ATCTCCAAAGCCAAAGGGCAGCCCCGAGAACCACAGGT |
| | | GTACACCCTGCCCCCATCCCGGGATGAGCTGACCAAGA |
| | | ACCAGGTCAGCCTGACCTGCCTGGTCAAAGGCTTCTATC |
| | | CCAGCGACATCGCCGTGGAGTGGGAGAGCAATGGGCAG |
| | | CCGGAGAACAACTACAAGACCACGCCTCCCGTGCTGGA |
| | | CTCCGACGGCTCCTTCTTCCTCTACAGCAAGCTCACCGT |
| | | GGACAAGAGCAGGTGGCAGCAGGGGAACGTCTTCTCAT |
| | | GCTCCGTGATGCATGAGGCTCTGCACAACCACTACACGC |
| | | AGAAGAGCCTCTCCCTGTCTCCGGGTAAA |
| 78 | nucleotide | see Table 4 |
| | sequence of | |
| | Germline control | |
| | (DP47) light | |
| | chain | |
| 82 | DP47 heavy chain | EVQLLESGGGLVQPGGSLRLSCAASGFTFSSYAMSWVRQA |
| | (hu IgG1 | PGKGLEWVSAISGSGGSTYYADSVKGRFTISRDNSKNTLYL |
| | PGLALA) | QMNSLRAEDTAVYYCAKGSGFDYWGQGTLVTVSSASTKG |
| | | PSVFPLAPSSKSTSGGTAALGCLVKDYFPEPVTVSWNSGAL |
| | | TSGVHTFPAVLQSSGLYSLSSVVTVPSSSLGTQTYICNVNH |
| | | KPSNTKVDKKVEPKSCDKTHTCPPCPAPEAAGGPSVFLFPP |
| | | KPKDTLMISRTPEVTCVVVDVSHEDPEVKFNWYVDGVEV |
| | | HNAKTKPREEQYNSTYRVVSVLTVLHQDWLNGKEYKCKV |
| | | SNKALGAPIEKTISKAKGQPREPQVYTLPPSRDELTKNQVS |
| | | LTCLVKGFYPSDIAVEWESNGQPENNYKTTPPVLDSDGSFF |
| | | LYSKLTVDKSRWQQGNVFSCSVMHEALHNHYTQKSLSLS |
| | | PGK |
| 80 | Germline control | see Table 4 |
| | (DP47) light | |
| | chain | |

Table 7 summarizes the yield and final monomer content of untargeted DP47 huIgG1 (Control F).

TABLE 7
Biochemical analysis of Control F

| | Monomer [%] | Yield |
|---|---|---|
| Construct | (SEC) | [mg/l] |
| Control F (germline DP47 human IgG1 | 100 | 50 |
| PGLALA | | |

### Example 2

### Preparation and Purification of Targeted huCMP Trimeric OX40 Ligand-Containing Antigen Binding Molecules

A targeted huCMP-trimeric OX40 ligand-containing antigen binding molecule was prepared similarly to the 4-1BBL-containing antigen binding molecule. For human OX40 ligand, the DNA sequence encoding part of the ectodomain (amino acids 51-183, SEQ ID NO:11) was synthetized according to P23510 (Uniprot Accession) and fused via a (G4S)₄ linker downstream of the trimerization domain. Two Asn-linked glycosylation sites (N90 and N114) were replaced by aspartic acid (Asp) by mutagenesis.

The chain encoding FAP-targeted OX40 ligand and the light chain with FAP specificity are similar to those shown for 4-1BBL in FIG. 1A. Disulfide bonds are formed between three FAP-targeted OX40 ligand chains leading to the formation of covalently linked CMP-trimeric OX40 ligand-containing antigen binding molecule, targeted to FAP.

Table 8 shows, respectively, the cDNA and amino acid sequences of the FAP-targeted human OX40 ligand-containing antigen binding molecule, which is trimerizing via huCMP trimerization domain.

TABLE 8
cDNA and amino acid sequences of FAP(28H1)-targeted
human CMP trimeric OX40L-containing antigen
binding molecule

| SEQ ID | | |
|---|---|---|
| NO: | Description | Sequence |
| 74 | nucleotide sequence of | GAAGTGCAGCTGCTGGAATCCGGCGGAGGCCTG |
| | FAP(28H1)(VHCH1)- | GTGCAGCCTGGCGGATCTCTGAGACTGTCCTGC |
| | CMPtd-Ox40L(51-183) | GCCGCCTCCGGCTTCACCTTCTCCTCCCACGCCA |
| | | TGTCCTGGGTCCGACAGGCTCCTGGCAAAGGCC |
| | | TGGAATGGGTGTCCGCCATCTGGGCCTCCGGCG |
| | | AGCAGTACTACGCCGACTCTGTGAAGGGCCGGT |
| | | TCACCATCTCCCGGGACAACTCCAAGAACACCC |
| | | TGTACCTGCAGATGAACTCCCTGCGGGCCGAGG |
| | | ACACCGCCGTGTACTACTGTGCCAAGGGCTGGC |
| | | TGGGCAACTTCGACTACTGGGGACAGGGCACCC |
| | | TGGTCACCGTGTCCAGCGCTAGCACAAAGGGAC |
| | | CTAGCGTGTTCCCCCTGGCCCCCAGCAGCAAGT |
| | | CTACATCTGGCGGAACAGCCGCCCTGGGCTGCC |
| | | TCGTGAAGGACTACTTTCCCGAGCCCGTGACCG |
| | | TGTCCTGGAACTCTGGCGCTCTGACAAGCGGCG |
| | | TGCACACCTTTCCAGCCGTGCTGCAGAGCAGCG |
| | | GCCTGTACTCTCTGAGCAGCGTCGTGACAGTGC |
| | | CCAGCAGCTCTCTGGGCACCCAGACCTACATCT |
| | | GCAACGTGAACCACAAGCCCAGCAACACCAAG |
| | | GTGGACAAGAAGGTGGAACCCAAGAGCTGCGA |
| | | CGGCGGAGGGGGATCTGGCGGCGGAGGATCCG |
| | | AGGAAGATCCTTGCGCCTGCGAGAGCCTCGTGA |
| | | AGTTCCAGGCCAAGGTGGAAGGACTGCTGCAG |
| | | GCCCTGACCCGGAAACTGGAAGCCGTGTCCAAG |
| | | CGGCTGGCCATCCTGGAAAACACCGTGGTGTCC |
| | | GGAGGCGGAGGATCTGGCGGCGGAGGAAGTGG |
| | | CGGAGGGGGATCTGGGGGAGGCGGATCTCAGG |
| | | TGTCCCACAGATACCCCCGGATCCAGAGCATCA |
| | | AGGTGCAGTTCACCGAGTACAAGAAAGAGAAG |
| | | GGCTTCATCCTGACCAGCCAGAAAGAGGACGA |
| | | GATCATGAAGGTGCAGGACAACAGCGTGATCAT |
| | | CAACTGCGACGGCTTCTACCTGATCAGCCTGAA |
| | | GGGCTACTTCAGCCAGGAAGTGGACATCAGCCT |
| | | GCACTACCAGAAGGACGAGGAACCCCTGTTCCA |
| | | GCTGAAGAAAGTGCGGAGCGTGAACAGCCTGA |
| | | TGGTGGCCAGCCTGACCTACAAGGACAAGGTGT |
| | | ACCTGAACGTGACCACCGACAACACCAGCCTGG |
| | | ACGACTTCCACGTGAACGGCGGCGAGCTGATCC |
| | | TGATTCACCAGAACCCCGGCGAGTTCTGCGTGC |
| | | TC |
| 75 | nucleotide sequence of | See Table 1 |
| | anti-FAP(28H1) light | |
| | chain | |
| 72 | FAP(VHCH1)-CMPtd- | EVQLLESGGGLVQPGGSLRLSCAASGFTFSSHAMS |
| | Ox40L(51-183) | WVRQAPGKGLEWVSAIWASGEQYYADSVKGRFT |
| | | ISRDNSKNTLYLQMNSLRAEDTAVYYCAKGWLG |
| | | NFDYWGQGTLVTVSSASTKGPSVFPLAPSSKSTSG |
| | | GTAALGCLVKDYFPEPVTVSWNSGALTSGVHTFP |
| | | AVLQSSGLYSLSSVVTVPSSSLGTQTYICNVNHKP |
| | | SNTKVDKKVEPKSCDGGGGSGGGGSEEDPCACES |
| | | LVKFQAKVEGLLQALTRKLEAVSKRLAILENTVV |
| | | SGGGGSGGGGSGGGGSGGGGSQVSHRYPRIQSIK |
| | | VQFTEYKKEKGFILTSQKEDEIMKVQDNSVIINCD |
| | | GFYLISLKGYFSQEVDISLHYQKDEEPLFQLKKVR |
| | | SVNSLMVASLTYKDKVYLNVTTDNTSLDDFHVN |
| | | GGELILIHQNPGEFCVL |
| 76 | Anti-FAP (28H1) light | See Table 1 |
| | chain | |

Production, purification and characterization of the FAP-targeted huCMP-trimeric Ox40 ligand-containing antigen binding molecule was performed as described above for the FAP-targeted human 4-1BB ligand-containing antigen binding molecule.

All used analytical methods confirmed homogeneous preparation of trimerized molecules. Table 9 summarizes yield and final monomer content of the FAP-targeted CMP-trimeric Ox40 ligand-containing antigen binding molecule.

TABLE 9
Biochemical analysis of FAP-targeted CMP-trimeric
OX40L-containing antigen binding molecule

| | Trimer [%] | CE-SDS non red |
|---|---|---|
| Construct | (SEC) | [%] |
| FAP-targeted CMP- | 85% | 89.4% |
| trimeric Ox40 ligand- | | (226 kDa) |
| containing antigen | | |
| binding molecule | | |

### Example 3

### Biochemical Characterization of FAP-Targeted huCMP Trimeric 4-1BB Ligand-Containing Antigen Binding Molecule by Surface Plasmon Resonance

### 3.1. Preparation, Purification and Characterization of 4-1BB

DNA sequences encoding the ectodomains of human, mouse or cynomolgus 4-1BB (Table 10) were subcloned in frame with the human IgG1 heavy chain CH2 and CH3 domains on the knob (Merchant et al., 1998). An AcTEV protease cleavage site was introduced between an antigen ectodomain and the Fc of human IgG1. An Avi tag for directed biotinylation was introduced at the C-terminus of the antigen-Fc knob. Combination of the antigen-Fc knob chain containing the S354C/T366W mutations, with a Fc hole chain containing the Y349C/T366S/L368A/Y407V mutations allows generation of a heterodimer which includes a single copy of the 4-1BB ectodomain containing chain, thus creating a monomeric form of Fc-linked antigen (FIG. 1C). Table 11 lists the cDNA and amino acid sequences of monomeric antigen Fc(kih) fusion molecules as depicted in FIG. 1C.

TABLE 10
Amino acid numbering of antigen ectodomains
(ECD) and their origin

| SEQ | | | |
|---|---|---|---|
| ID NO: | Construct | Origin | ECD |
| 83 | human 4-1BB | Synthetized according to | aa 24-186 |
| | ECD | Q07011 | |
| 84 | cynomolgus 4-1BB | isolated from cynomolgus | aa 24-186 |
| | ECD | blood | |
| 85 | murine 4-1BB | Synthetized according to | aa 24-187 |
| | ECD | P20334 | |

TABLE 11
cDNA and Amino acid sequences of monomeric antigen
Fc(kih) fusion molecules (produced by combination of
one Fc hole chain with one antigen Fc knob chain)

| SEQ ID | | |
|---|---|---|
| NO: | Antigen | Sequence |
| 86 | Nucleotide | GACAAAACTCACACATGCCCACCGTGCCCAGCACCTGA |
| | sequence of Fc | ACTCCTGGGGGGACCGTCAGTCTTCCTCTTCCCCCCAAA |
| | hole chain | ACCCAAGGACACCCTCATGATCTCCCGGACCCCTGAGGT |
| | | CACATGCGTGGTGGTGGACGTGAGCCACGAAGACCCTG |
| | | AGGTCAAGTTCAACTGGTACGTGGACGGCGTGGAGGTG |
| | | CATAATGCCAAGACAAAGCCGCGGGAGGAGCAGTACAA |
| | | CAGCACGTACCGTGTGGTCAGCGTCCTCACCGTCCTGCA |
| | | CCAGGACTGGCTGAATGGCAAGGAGTACAAGTGCAAGG |
| | | TCTCCAACAAAGCCCTCCCAGCCCCCATCGAGAAAACC |
| | | ATCTCCAAAGCCAAAGGGCAGCCCCGAGAACCACAGGT |
| | | GTGCACCCTGCCCCCATCCCGGGATGAGCTGACCAAGA |
| | | ACCAGGTCAGCCTCTCGTGCGCAGTCAAAGGCTTCTATC |
| | | CCAGCGACATCGCCGTGGAGTGGGAGAGCAATGGGCAG |
| | | CCGGAGAACAACTACAAGACCACGCCTCCCGTGCTGGA |
| | | CTCCGACGGCTCCTTCTTCCTCGTGAGCAAGCTCACCGT |
| | | GGACAAGAGCAGGTGGCAGCAGGGGAACGTCTTCTCAT |
| | | GCTCCGTGATGCATGAGGCTCTGCACAACCACTACACGC |
| | | AGAAGAGCCTCTCCCTGTCTCCGGGTAAA |
| 87 | Nucleotide | CTGCAGGACCCCTGCAGCAACTGCCCTGCCGGCACCTTC |
| | sequence of | TGCGACAACAACCGGAACCAGATCTGCAGCCCCTGCCC |
| | human 4-1BB | CCCCAACAGCTTCAGCTCTGCCGGCGGACAGCGGACCT |
| | antigen Fc knob | GCGACATCTGCAGACAGTGCAAGGGCGTGTTCAGAACC |
| | chain | CGGAAAGAGTGCAGCAGCACCAGCAACGCCGAGTGCGA |
| | | CTGCACCCCCGGCTTCCATTGTCTGGGAGCCGGCTGCAG |
| | | CATGTGCGAGCAGGACTGCAAGCAGGGCCAGGAACTGA |
| | | CCAAGAAGGGCTGCAAGGACTGCTGCTTCGGCACCTTC |
| | | AACGACCAGAAGCGGGGCATCTGCCGGCCCTGGACCAA |
| | | CTGTAGCCTGGACGGCAAGAGCGTGCTGGTCAACGGCA |
| | | CCAAAGAACGGGACGTCGTGTGCGGCCCCAGCCCTGCT |
| | | GATCTGTCTCCTGGGGCCAGCAGCGTGACCCCTCCTGCC |
| | | CCTGCCAGAGAGCCTGGCCACTCTCCTCAGGTCGACGAA |
| | | CAGTTATATTTTCAGGGCGGCTCACCCAAATCTGCAGAC |
| | | AAAACTCACACATGCCCACCGTGCCCAGCACCTGAACTC |
| | | CTGGGGGGACCGTCAGTCTTCCTCTTCCCCCCAAAACCC |
| | | AAGGACACCCTCATGATCTCCCGGACCCCTGAGGTCACA |
| | | TGCGTGGTGGTGGACGTGAGCCACGAAGACCCTGAGGT |
| | | CAAGTTCAACTGGTACGTGGACGGCGTGGAGGTGCATA |
| | | ATGCCAAGACAAAGCCGCGGGAGGAGCAGTACAACAGC |
| | | ACGTACCGTGTGGTCAGCGTCCTCACCGTCCTGCACCAG |
| | | GACTGGCTGAATGGCAAGGAGTACAAGTGCAAGGTCTC |
| | | CAACAAAGCCCTCCCAGCCCCCATCGAGAAAACCATCT |
| | | CCAAAGCCAAAGGGCAGCCCCGAGAACCACAGGTGTAC |
| | | ACCCTGCCCCCATGCCGGGATGAGCTGACCAAGAACCA |
| | | GGTCAGCCTGTGGTGCCTGGTCAAAGGCTTCTATCCCAG |
| | | CGACATCGCCGTGGAGTGGGAGAGCAATGGGCAGCCGG |
| | | AGAACAACTACAAGACCACGCCTCCCGTGCTGGACTCC |
| | | GACGGCTCCTTCTTCCTCTACAGCAAGCTCACCGTGGAC |
| | | AAGAGCAGGTGGCAGCAGGGGAACGTCTTCTCATGCTC |
| | | CGTGATGCATGAGGCTCTGCACAACCACTACACGCAGA |
| | | AGAGCCTCTCCCTGTCTCCGGGTAAATCCGGAGGCCTGA |
| | | ACGACATCTTCGAGGCCCAGAAGATTGAATGGCACGAG |
| 88 | Nucleotide | TTGCAGGATCTGTGTAGTAACTGCCCAGCTGGTACATTC |
| | sequence of | TGTGATAATAACAGGAGTCAGATTTGCAGTCCCTGTCCT |
| | cynomolgus 4- | CCAAATAGTTTCTCCAGCGCAGGTGGACAAAGGACCTGT |
| | 1BB antigen | GACATATGCAGGCAGTGTAAAGGTGTTTTCAAGACCAG |
| | Fc knob chain | GAAGGAGTGTTCCTCCACCAGCAATGCAGAGTGTGACT |
| | | GCATTTCAGGGTATCACTGCCTGGGGGCAGAGTGCAGC |
| | | ATGTGTGAACAGGATTGTAAACAAGGTCAAGAATTGAC |
| | | AAAAAAAGGTTGTAAAGACTGTTGCTTTGGGACATTTAA |
| | | TGACCAGAAACGTGGCATCTGTCGCCCCTGGACAAACT |
| | | GTTCTTTGGATGGAAAGTCTGTGCTTGTGAATGGGACGA |
| | | AGGAGAGGGACGTGGTCTGCGGACCATCTCCAGCCGAC |
| | | CTCTCTCCAGGAGCATCCTCTGCGACCCCGCCTGCCCCT |
| | | GCGAGAGAGCCAGGACACTCTCCGCAGGTCGACGAACA |
| | | GTTATATTTTCAGGGCGGCTCACCCAAATCTGCAGACAA |
| | | AACTCACACATGCCCACCGTGCCCAGCACCTGAACTCCT |
| | | GGGGGGACCGTCAGTCTTCCTCTTCCCCCCAAAACCCAA |
| | | GGACACCCTCATGATCTCCCGGACCCCTGAGGTCACATG |
| | | CGTGGTGGTGGACGTGAGCCACGAAGACCCTGAGGTCA |
| | | AGTTCAACTGGTACGTGGACGGCGTGGAGGTGCATAAT |
| | | GCCAAGACAAAGCCGCGGGAGGAGCAGTACAACAGCA |
| | | CGTACCGTGTGGTCAGCGTCCTCACCGTCCTGCACCAGG |
| | | ACTGGCTGAATGGCAAGGAGTACAAGTGCAAGGTCTCC |
| | | AACAAAGCCCTCCCAGCCCCCATCGAGAAAACCATCTC |
| | | CAAAGCCAAAGGGCAGCCCCGAGAACCACAGGTGTACA |
| | | CCCTGCCCCCATGCCGGGATGAGCTGACCAAGAACCAG |
| | | GTCAGCCTGTGGTGCCTGGTCAAAGGCTTCTATCCCAGC |
| | | GACATCGCCGTGGAGTGGGAGAGCAATGGGCAGCCGGA |
| | | GAACAACTACAAGACCACGCCTCCCGTGCTGGACTCCG |
| | | ACGGCTCCTTCTTCCTCTACAGCAAGCTCACCGTGGACA |
| | | AGAGCAGGTGGCAGCAGGGGAACGTCTTCTCATGCTCC |
| | | GTGATGCATGAGGCTCTGCACAACCACTACACGCAGAA |
| | | GAGCCTCTCCCTGTCTCCGGGTAAATCCGGAGGCCTGAA |
| | | CGACATCTTCGAGGCCCAGAAGATTGAATGGCACGAG |
| 89 | Nucleotide | GTGCAGAACAGCTGCGACAACTGCCAGCCCGGCACCTT |
| | sequence of | CTGCCGGAAGTACAACCCCGTGTGCAAGAGCTGCCCCC |
| | murine 4-1BB | CCAGCACCTTCAGCAGCATCGGCGGCCAGCCCAACTGC |
| | antigen Fc knob | AACATCTGCAGAGTGTGCGCCGGCTACTTCCGGTTCAAG |
| | chain | AAGTTCTGCAGCAGCACCCACAACGCCGAGTGCGAGTG |
| | | CATCGAGGGCTTCCACTGCCTGGGCCCCCAGTGCACCAG |
| | | ATGCGAGAAGGACTGCAGACCCGGCCAGGAACTGACCA |
| | | AGCAGGGCTGTAAGACCTGCAGCCTGGGCACCTTCAAC |
| | | GACCAGAACGGGACCGGCGTGTGCCGGCCTTGGACCAA |
| | | TTGCAGCCTGGACGGGAGAAGCGTGCTGAAAACCGGCA |
| | | CCACCGAGAAGGACGTCGTGTGCGGCCCTCCCGTGGTGT |
| | | CCTTCAGCCCTAGCACCACCATCAGCGTGACCCCTGAAG |
| | | GCGGCCCTGGCGGACACTCTCTGCAGGTCCTGGTCGACG |
| | | AACAGTTATATTTTCAGGGCGGCTCACCCAAATCTGCAG |
| | | ACAAAACTCACACATGCCCACCGTGCCCAGCACCTGAA |
| | | CTCCTGGGGGGACCGTCAGTCTTCCTCTTCCCCCCAAAA |
| | | CCCAAGGACACCCTCATGATCTCCCGGACCCCTGAGGTC |
| | | ACATGCGTGGTGGTGGACGTGAGCCACGAAGACCCTGA |
| | | GGTCAAGTTCAACTGGTACGTGGACGGCGTGGAGGTGC |
| | | ATAATGCCAAGACAAAGCCGCGGGAGGAGCAGTACAAC |
| | | AGCACGTACCGTGTGGTCAGCGTCCTCACCGTCCTGCAC |
| | | CAGGACTGGCTGAATGGCAAGGAGTACAAGTGCAAGGT |
| | | CTCCAACAAAGCCCTCCCAGCCCCCATCGAGAAAACCA |
| | | TCTCCAAAGCCAAAGGGCAGCCCCGAGAACCACAGGTG |
| | | TACACCCTGCCCCCATGCCGGGATGAGCTGACCAAGAA |
| | | CCAGGTCAGCCTGTGGTGCCTGGTCAAAGGCTTCTATCC |
| | | CAGCGACATCGCCGTGGAGTGGGAGAGCAATGGGCAGC |
| | | CGGAGAACAACTACAAGACCACGCCTCCCGTGCTGGAC |
| | | TCCGACGGCTCCTTCTTCCTCTACAGCAAGCTCACCGTG |
| | | GACAAGAGCAGGTGGCAGCAGGGGAACGTCTTCTCATG |
| | | CTCCGTGATGCATGAGGCTCTGCACAACCACTACACGCA |
| | | GAAGAGCCTCTCCCTGTCTCCGGGTAAATCCGGAGGCCT |
| | | GAACGACATCTTCGAGGCCCAGAAGATTGAATGGCACG |
| | | AG |
| 90 | Fc hole chain | DKTHTCPPCPAPELLGGPSVFLFPPKPKDTLMISRTPEVTCV |
| | | VVDVSHEDPEVKFNWYVDGVEVHNAKTKPREEQYNSTYR |
| | | VVSVLTVLHQDWLNGKEYKCKVSNKALPAPIEKTISKAKG |
| | | QPREPQVCTLPPSRDELTKNQVSLSCAVKGFYPSDIAVEWE |
| | | SNGQPENNYKTTPPVLDSDGSFFLVSKLTVDKSRWQQGNV |
| | | FSCSVMHEALHNHYTQKSLSLSPGK |
| 91 | human 4-1BB | LQDPCSNCPAGTFCDNNRNQICSPCPPNSFSSAGGQRTCDI |
| | antigen Fc knob | CRQCKGVFRTRKECSSTSNAECDCTPGFHCLGAGCSMCEQ |
| | chain | DCKQGQELTKKGCKDCCFGTFNDQKRGICRPWTNCSLDG |
| | | KSVLVNGTKERDVVCGPSPADLSPGASSVTPPAPAREPGHS |
| | | PQVDEQLYFQGGSPKSADKTHTCPPCPAPELLGGPSVFLFP |
| | | PKPKDTLMISRTPEVTCVVVDVSHEDPEVKFNWYVDGVE |
| | | VHNAKTKPREEQYNSTYRVVSVLTVLHQDWLNGKEYKCK |
| | | VSNKALPAPIEKTISKAKGQPREPQVYTLPPCRDELTKNQV |
| | | SLWCLVKGFYPSDIAVEWESNGQPENNYKTTPPVLDSDGS |
| | | FFLYSKLTVDKSRWQQGNVFSCSVMHEALHNHYTQKSLS |
| | | LSPGKSGGLNDIFEAQKIEWHE |
| 92 | cynomolgus 4- | LQDLCSNCPAGTFCDNNRSQICSPCPPNSFSSAGGQRTCDIC |
| | 1BB antigen | RQCKGVFKTRKECSSTSNAECDCISGYHCLGAECSMCEQD |
| | Fc knob chain | CKQGQELTKKGCKDCCFGTFNDQKRGICRPWTNCSLDGK |
| | | SVLVNGTKERDVVCGPSPADLSPGASSATPPAPAREPGHSP |
| | | QVDEQLYFQGGSPKSADKTHTCPPCPAPELLGGPSVFLFPP |
| | | KPKDTLMISRTPEVTCVVVDVSHEDPEVKFNWYVDGVEV |
| | | HNAKTKPREEQYNSTYRVVSVLTVLHQDWLNGKEYKCKV |
| | | SNKALPAPIEKTISKAKGQPREPQVYTLPPCRDELTKNQVS |
| | | LWCLVKGFYPSDIAVEWESNGQPENNYKTTPPVLDSDGSF |
| | | FLYSKLTVDKSRWQQGNVFSCSVMHEALHNHYTQKSLSL |
| | | SPGKSGGLNDIFEAQKIEWHE |
| 93 | murine 4-1BB | VQNSCDNCQPGTFCRKYNPVCKSCPPSTFSSIGGQPNCNIC |
| | antigen Fc knob | RVCAGYFRFKKFCSSTHNAECECIEGFHCLGPQCTRCEKDC |
| | chain | RPGQELTKQGCKTCSLGTFNDQNGTGVCRPWTNCSLDGR |
| | | SVLKTGTTEKDVVCGPPVVSFSPSTTISVTPEGGPGGHSLQ |
| | | VLVDEQLYFQGGSPKSADKTHTCPPCPAPELLGGPSVFLFP |
| | | PKPKDTLMISRTPEVTCVVVDVSHEDPEVKFNWYVDGVE |
| | | VHNAKTKPREEQYNSTYRVVSVLTVLHQDWLNGKEYKCK |
| | | VSNKALPAPIEKTISKAKGQPREPQVYTLPPCRDELTKNQV |
| | | SLWCLVKGFYPSDIAVEWESNGQPENNYKTTPPVLDSDGS |
| | | FFLYSKLTVDKSRWQQGNVFSCSVMHEALHNHYTQKSLS |
| | | LSPGKSGGLNDIFEAQKIEWHE |

All 4-1BB-Fc-fusion encoding sequences were cloned into a plasmid vector, which drives expression of the insert from an MPSV promoter and contains a synthetic polyA signal sequence located at the 3′ end of the CDS. In addition, the vector contains an EBV OriP sequence for episomal maintenance of the plasmid.

For preparation of the biotinylated monomeric antigen/Fc fusion molecules, exponentially growing suspension HEK293 EBNA cells were co-transfected with three vectors encoding the two components of fusion protein (knob and hole chains) as well as BirA, an enzyme necessary for the biotinylation reaction. The corresponding vectors were used at a 1:1:0.05 ratio (“antigen ECD-AcTEV-Fc knob”:“Fc hole”:“BirA”).

For protein production in 500 ml shake flasks, 400 million HEK293 EBNA cells were seeded 24 hours before transfection. For transfection cells were centrifuged for 5 minutes at 210 g, and supernatant was replaced by pre-warmed CD CHO medium. Expression vectors were resuspended in 20 mL of CD CHO medium containing 200 μg of vector DNA. After addition of 540 μL of polyethylenimine (PEI), the solution was vortexed for 15 seconds and incubated for 10 minutes at room temperature. Afterwards, cells were mixed with the DNA/PEI solution, transferred to a 500 mL shake flask and incubated for 3 hours at 37° C. in an incubator with a 5% CO₂ atmosphere. After the incubation, 160 mL of F17 medium was added and cells were cultured for 24 hours. One day after transfection, 1 mM valproic acid and 7% Feed were added to the culture. After 7 days of culturing, the cell supernatant was collected by spinning down cells for 15 min at 210 g. The solution was sterile filtered (0.22 μm filter), supplemented with sodium azide to a final concentration of 0.01% (w/v), and kept at 4° C.

Secreted proteins were purified from cell culture supernatants by affinity chromatography using Protein A, followed by size exclusion chromatography. For affinity chromatography, the supernatant was loaded on a HiTrap ProteinA HP column (CV=5 mL, GE Healthcare) equilibrated with 40 mL 20 mM sodium phosphate, 20 mM sodium citrate pH 7.5. Unbound protein was removed by washing with at least 10 column volumes of 20 mM sodium phosphate, 20 mM sodium citrate, 0.5 M sodium chloride containing buffer (pH 7.5). The bound protein was eluted using a linear pH-gradient of sodium chloride (from 0 to 500 mM) created over 20 column volumes of 20 mM sodium citrate, 0.01% (v/v) Tween-20, pH 2.5. The column was then washed with 10 column volumes of 20 mM sodium citrate, 500 mM sodium chloride, 0.01% (v/v) Tween-20, pH 2.5.

The pH of collected fractions was adjusted by adding 1/40 (v/v) of 2M Tris, pH8.0. The protein was concentrated and filtered prior to loading on a HiLoad Superdex 200 column (GE Healthcare) equilibrated with 2 mM MOPS, 150 mM sodium chloride, 0.02% (w/v) sodium azide solution of pH 7.4.

### 3.2. Biochemical Characterization by Surface Plasmon Resonance

Binding of FAP-targeted huCMP-trimeric 4-1BB ligand-containing antigen binding molecule to the recombinant 4-1BB Fc (kih) was assessed by surface plasmon resonance (SPR). All SPR experiments were performed on a Biacore T100 at 25° C. with HBS-EP as running buffer (0.01 M HEPES pH 7.4, 0.15 M NaCl, 3 mM EDTA, 0.005% Surfactant P20, Biacore, Freiburg/Germany).

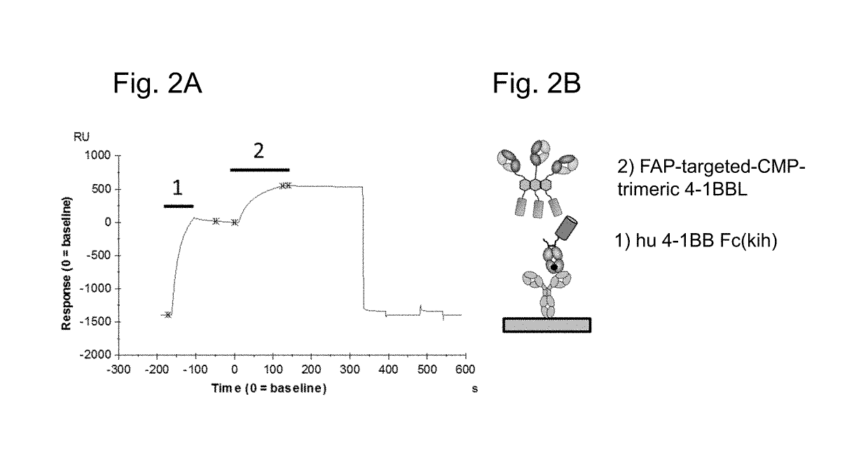

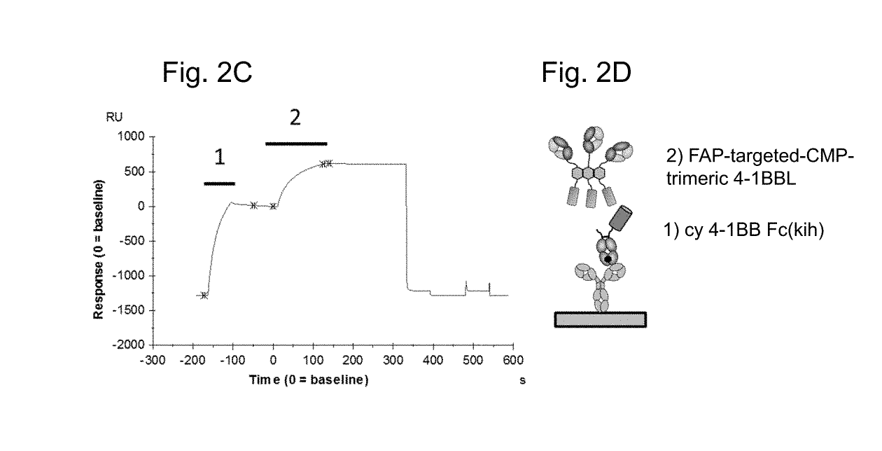

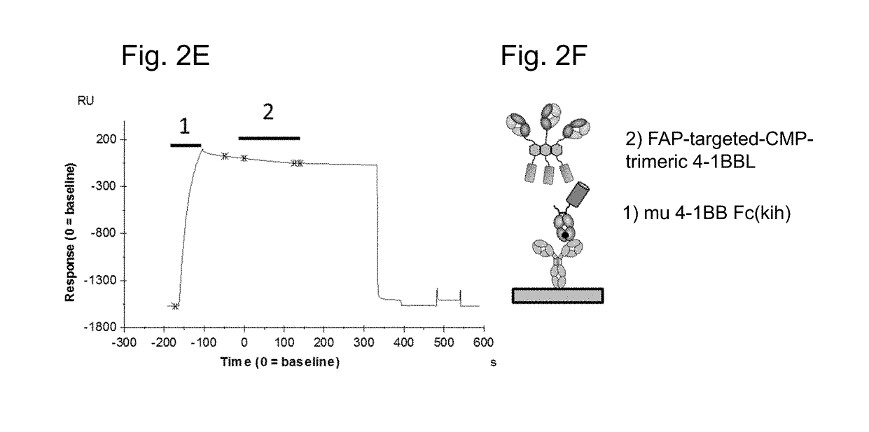

The avidity of the interaction between the huCMP-trimeric human 4-1BB ligand-containing antigen binding molecule and recombinant 4-1BB (human, cyno and murine) was determined as described below and shown in FIGS. 2A to 2F.

Anti-human Fc antibody (Biacore, Freiburg/Germany) was directly coupled on a CM5 chip at pH 5.0 using the standard amine coupling kit (Biacore, Freiburg/Germany). The immobilization level was about 6800 RU. Recombinant human, cynomolgus and murine 4-1BB Fc(kih) were captured for 60 seconds at 200 nM. FAP-targeted CMP-trimeric 4-1BB ligand-containing antigen binding molecule was passed at 200 nM with a flow of 30 μL/minutes through the flow cells over 120 seconds. The dissociation was monitored for 120 seconds. Bulk refractive index differences were corrected for by subtracting the response obtained on reference flow cell. Here, the FAP-targeted CMP-trimeric 4-1BB ligand-containing antigen binding molecule was flown over a surface with immobilized anti-human Fc antibody but on which HBS-EP has been injected rather than recombinant human, cynomolgus and murine 4-1BB Fc(kih).

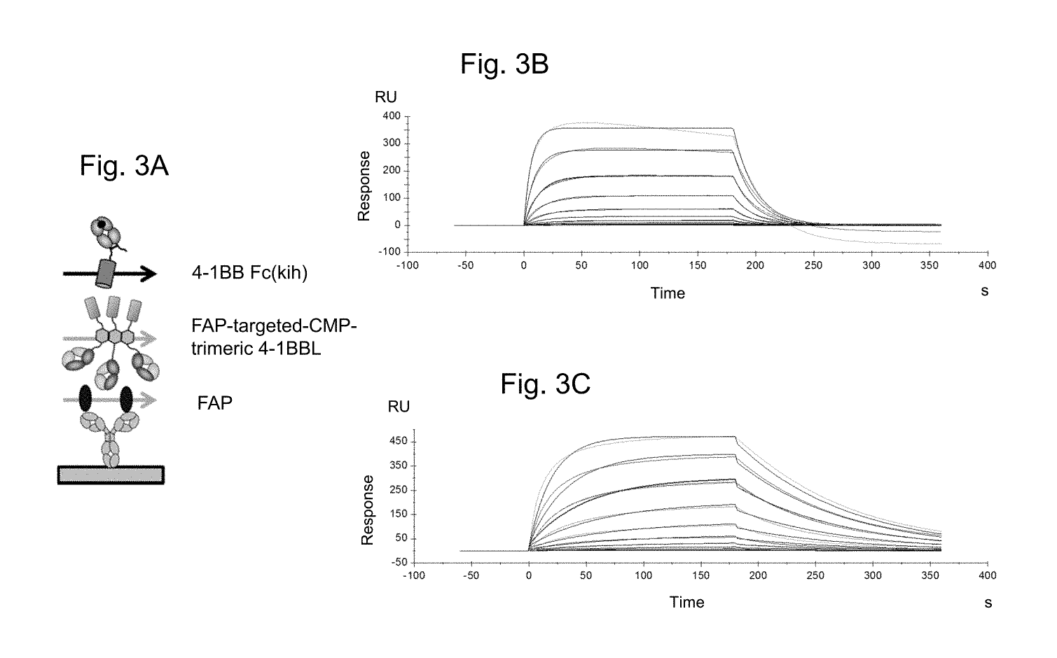

The affinity of the interaction between FAP-targeted huCMP-trimeric 4-1BB ligand-containing antigen binding molecule and recombinant human and cynomolgus 4-1BB was determined as described below and in FIGS. 3A to 3C.

Anti-penta his antibody (Qiagen) was directly coupled on a CM5 chip at pH 5.0 using the standard amine coupling kit (Biacore, Freiburg/Germany). The immobilization level was about 9200 RU. FAP was captured for 90 seconds at 200 nM. FAP-targeted CMP-trimeric 4-1BB ligand-containing antigen binding molecule was passed at 100 nM through the flow cells over 120 seconds. Recombinant human, cynomolgus and murine 4-1BB Fc(kih) were passed at a concentration range from 0.49 to 1000 nM with a flow of 30 μL/minutes through the flow cells over 180 seconds. The dissociation was monitored for 180 seconds. Bulk refractive index differences were corrected for by subtracting the response obtained on reference flow cell. Here, recombinant 4-1BB Fc(kih) was flown over a surface with immobilized anti-penta His antibody but on which HBS-EP has been injected rather than FAP and targeted huCMP-trimeric 4-1BB ligand.

Kinetic constants were derived using the Biacore T100 Evaluation Software (vAA, Biacore AB, Uppsala/Sweden), to fit rate equations for 1:1 Langmuir binding by numerical integration (Table 12).

TABLE 12
Affinity constants determined by fitting
rate equations for 1:1 Langmuir binding

| | | Kₒₙ | K_{off} | K_{D} |
|---|---|---|---|---|
| Ligand | Analyte | (1/Ms) | (1/s) | (M) |
| FAP-targeted huCMP- | hu4-1BB | 1.1E+05 | 5.2E−02 | 4.8E−07 |
| trimeric 4-1BB | Fc(kih) | | | |
| ligand-containing | | | | |
| antigen binding molecule | | | | |
| FAP-targeted huCMP- | cy4-1BB | 3.9E+04 | 1.0E−02 | 2.7E−07 |
| trimeric 4-1BB | Fc(kih) | | | |
| ligand-containing | | | | |
| antigen binding molecule | | | | |

### Example 4

### Functional Characterization of Targeted huCMP Trimeric 4-1BB Ligand-Containing Antigen Binding Molecules

4.1. Binding on 4-1BB Expressing Cells: Binding on Resting (Naïve) and Activated Human PBMCs

Buffy coats were obtained from the Zurich blood donation center. To isolate fresh peripheral blood mononuclear cells (PBMCs) the buffy coat was diluted with the same volume of DPBS (Gibco by Life Technologies, Cat. No. 14190 326). 50 mL polypropylene centrifuge tubes (TPP, Cat.-No. 91050) were supplied with 15 mL Histopaque 1077 (SIGMA Life Science, Cat.-No. 10771, polysucrose and sodium diatrizoate, adjusted to a density of 1.077 g/mL) and the diluted buffy coat solution was layered above the Histopaque 1077. The tubes were centrifuged for 30 min at 400×g. PBMCs were then collected from the interface, washed three times with DPBS and resuspended in T cell medium consisting of RPMI 1640 medium (Gibco by Life Technology, Cat. No. 42401-042) supplied with 10% Fetal Bovine Serum (FBS, Gibco by Life Technology, Cat. No. 16000-044, Lot 941273, gamma-irradiated, mycoplasma-free and heat inactivated at 56° C. for 35 min), 1% (v/v) GlutaMAX-I (GIBCO by Life Technologies, Cat. No. 35050 038), 1 mM Sodium Pyruvate (SIGMA, Cat. No. S8636), 1% (v/v) MEM non-essential amino acids (SIGMA, Cat.-No. M7145) and 50 μM β-Mercaptoethanol (SIGMA, M3148).

PBMCs were used directly after isolation (naïve cells) or stimulated to induce 4-1BB expression at the cell surface of T cells by culturing for 4 days in T cell medium supplemented with 200 U/mL Proleukin (Novartis Pharma Schweiz AG, CHCLB-P-476-700-10340) and 2 ug/mL PHA-L (SIGMA Cat.-No. L2769) in a 6-well tissue culture plate and then 1 day in a 6-well tissue culture plate coated with 10 ug/mL anti-human CD3 (clone OKT3, BioLegend, Cat.-No. 317315) and 2 ug/mL anti-human CD28 (clone CD28.2, BioLegend, Cat.-No.: 302928) in T cell medium supplied with 200 U/mL Proleukin at 37° C. and 5% CO₂.

To determine binding of huCMP trimeric 4-1BB ligand-containing antigen binding molecules to human PBMCs, 0.1×10⁶ naïve or activated PBMCs were added to each well of a round-bottom suspension cell 96-well plates (Greiner bio-one, cellstar, Cat. No. 650185). Plates were centrifuged 4 minutes with 400×g and at 4° C. and supernatant was discarded. If cells were fixed before acquisition by flow cytometry they were washed once with 200 μL/well DPBS and then stained for 30 min at 4° C. with 100 μL/mL DPBS containing 1:1000 diluted LIVE/DEAD Fixable Aqua Dead Cell Stain (Life technologies, Molecular Probes, Cat-No.: L34957). If cells were acquired the same day and DAPI was used to discriminate dead cells this staining step was skipped. Afterwards cells were washed once with 200 μL cold FACS buffer (DPBS supplied with 2% (v/v) FBS, 5 mM EDTA pH8 (Amresco, Cat. No. E177) and 7.5 mM sodium azide (Sigma-Aldrich S2002)). Next, 50 μL/well of 4° C. cold FACS buffer containing FAP-targeted CMP trimeric 4-1BB ligand-containing antigen binding molecule, DP47-targeted CMP trimeric 4-1BB ligand-containing antigen binding molecule or not-specific binding huIgG1 P329G LALA control antibody were added and cells were incubated for 120 minutes at 4° C. Cells were washed four times with 200 μL/well 4° C. FACS buffer. Afterwards cells were further stained with 50 μL/well of 4° C. cold FACS buffer containing 0.25 μg/mL anti-human CD4-BV421 (clone RPA-T4, mouse IgG1κ, BioLegend, Cat.-No. 300532), 0.25 μL anti-human CD8a-APC (clone RPA-T8, mouse IgG1κ, BD Pharmingen, Cat.-No. 555369) or 1:200 diluted anti-human CD8a-AF488 (clone RPA-T8, mouse IgG1κ, BD Pharmingen, Cat.-No. 557704), 0.12 μg/mL anti-human CD56-FITC (clone NCAM16.2, mouse IgG2bκ, BD Pharmingen, Cat.-No. 345811) and 2.5 μg/mL PE-conjugated AffiniPure anti-human IgG F(ab′)2-fragment-specific goat F(ab′)2 fragment (Jackson ImmunoResearch, Cat. No. 109 116 097) and incubated for 30 minutes at 4° C. Cells were washed twice with 200 uL FACS buffer/well. If cells were stained with LIVE/DEAD Fixable Aqua Dead Cell Stain, they were fixed with 50 μL/well DPBS containing 1% Formaldehyde (Sigma, HT501320-9.5L) and stored overnight at 4° C. Cells were resuspended in FACS buffer and acquired the next day using a 5-laser LSR-Fortessa (BD Bioscience with DIVA software). If DAPI staining was used to detect dead cells, cells were resuspended in 80 μL/well FACS buffer containing 0.2 μg/mL DAPI (Santa Cruz Biotec, Cat. No. Sc-3598) and acquired the same day using a 5-laser LSR-Fortessa (BD Bioscience with DIVA software).

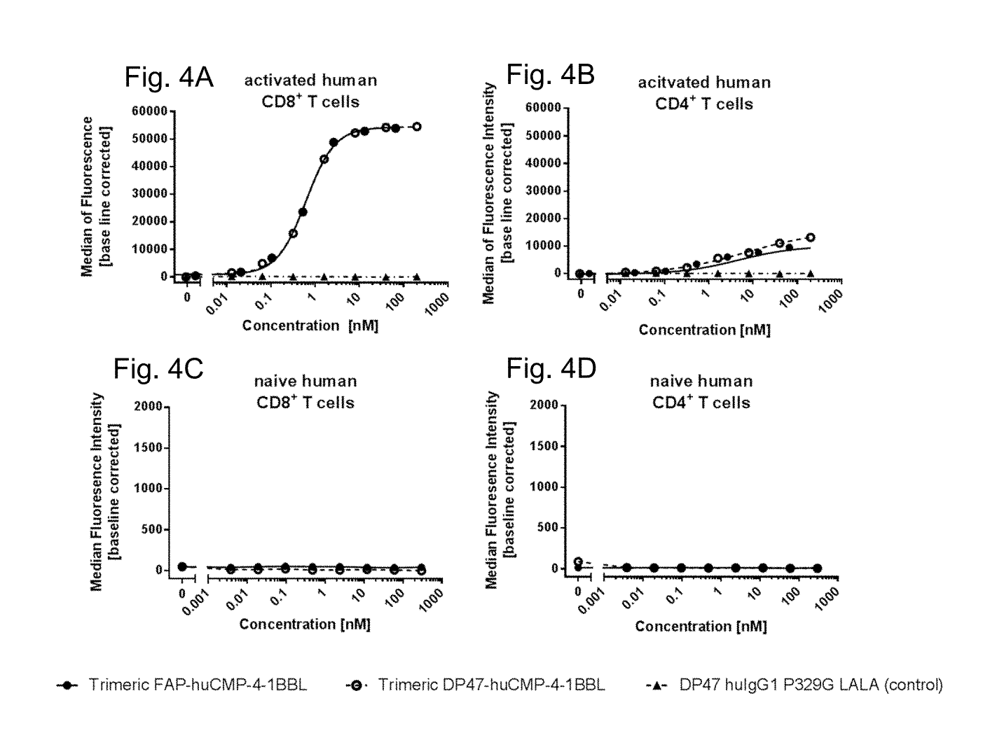

As shown in FIG. 4, neither FAP-targeted huCMP trimeric 4-1BB ligand-containing antigen binding molecules (trimeric FAP-huCMP-4-1BBL, filled circles) nor untargeted (DP47-targeted) huCMP-trimeric 4-1BB ligand-containing antigen binding molecules (trimeric DP47-huCMP-4-1BBL) did bind to resting (naïve) human CD4⁺ T-cells or CD8⁺ T-cells. In contrast, both constructs bound strongly to activated CD8⁺ or CD4⁺ T-cells, although the latter showed approximately 6-fold lower intensity of specific fluorescence as compared to CD8⁺ T-cells. This is consistent with the fact, that human 4-1BB is normally 10-20 fold higher expressed on CD8⁺ T cells compared to CD4⁺ T cells (depends on donor and activation protocol).

4.2. Binding on FAP-Expressing Tumor Cells

For binding assays on FAP expressing cells, the NIH/3T3-huFAP clone 39 cell line was used. To generate this cell line, NIH/3T3 cells were transfected with human FAP (NIH/3T3-huFAP clone 39). The cells were generated by transfection of mouse embryonic fibroblast NIH/3T3 cells (ATCC CRL-1658) with the expression pETR4921 plasmid encoding human FAP under a CMV promoter. Cells were maintained in the presence of 1.5 μg/mL puromycin (InvivoGen, Cat.-No.: ant-pr-5). 0.1×10⁶ of FAP expressing tumor cells were added to each well of a round-bottom suspension cell 96-well plates (Greiner bio-one, cellstar, Cat.-No. 650185). Cells were washed once with 200 μL/well 4° C. cold FACS buffer and resuspended in 50 μL/well of 4° C. cold FACS buffer containing different concentrations of titrated FAP-targeted or DP47-targeted CMP-trimeric 4-1BB ligand-containing antigen binding molecule. Resuspended cells were incubated for 2 hour at 4° C. After washing four times with 200 μL/well FACS buffer, cells were stained with 50 μL/well of 4° C. cold FACS buffer containing either 68 μg/mL polyclonal Fluorescein (FITC)-conjugated AffiniPure Fab Fragment Goat Anti-Human IgG (H+L) or 0.25 ug/mL monoclonal Fluorescein (FITC)-conjugated anti-human IgG-specific mouse IgGiK (clone G18-145) or 8.3 ug/mL monoclonal phycoerythrin (PE)-labeled anti-human 4-1BB ligand mouse IgG2b (Clone 282220) for 1 h at 4° C. Cells were washed twice with 200 μL/well cold FACS buffer and resuspended in 100 μL/well FACS buffer containing 0.2 μg/mL DAPI (Santa Cruz Biotec, Cat. No. Sc-3598) and acquired the same day using a 5-laser LSR-Fortessa (BD Bioscience with DIVA software).

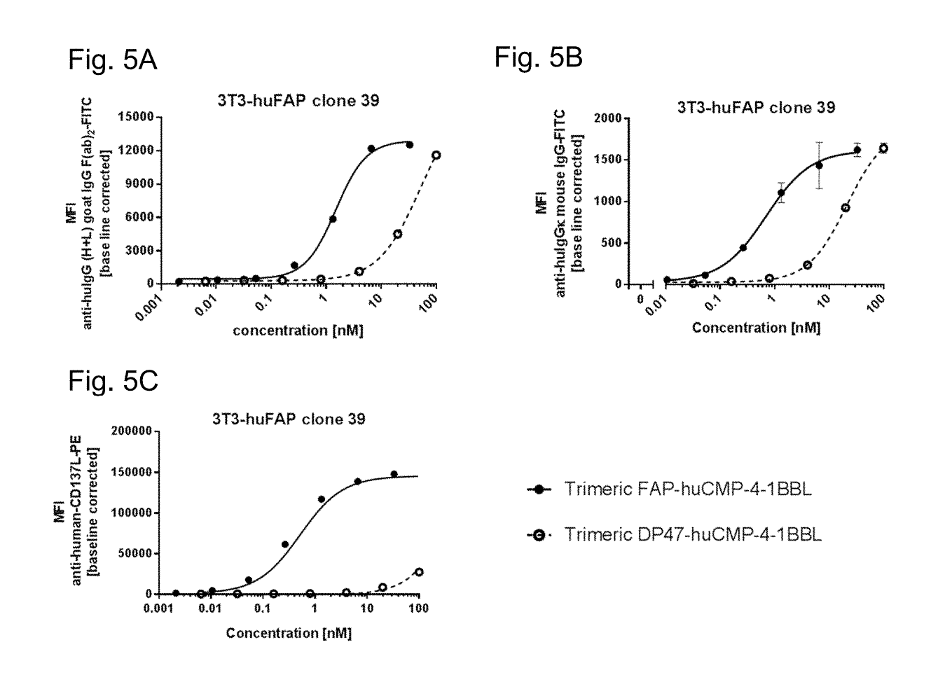

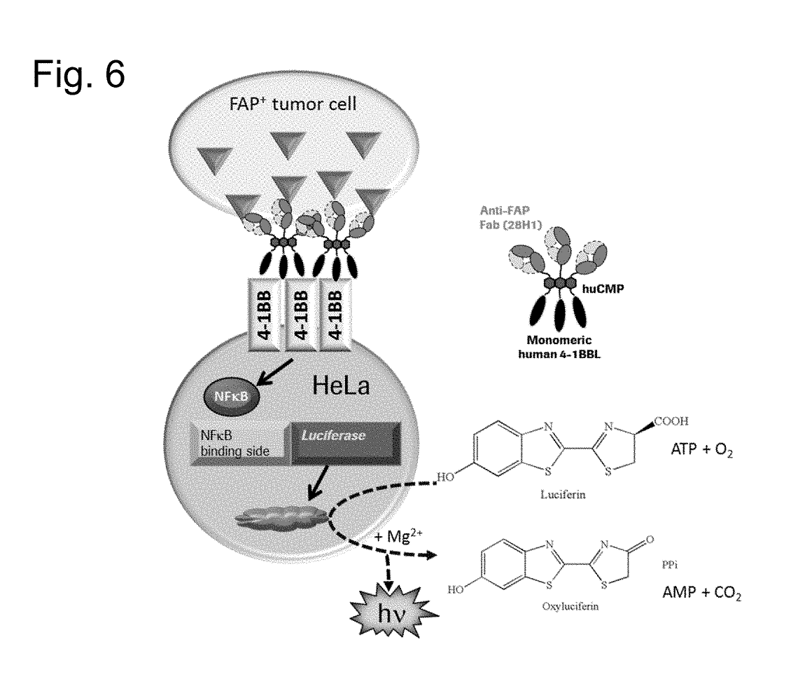

As shown in FIG. 5, the FAP-targeted CMP-trimeric 4-1BB ligand-containing antigen binding molecule, but not the untargeted, DP47-Fab-containing construct (DP47-targeted CMP-trimeric 4-1BB ligand), efficiently bound to human FAP-expressing cells. At concentrations above 10 nM the DP47-targeted CMP-trimeric 4-1BB ligand-containing antigen binding molecule shows unspecific binding if detected with anti-huIgG (H+L) or anti-huIgGκ-specific antibodies.

### Example 5

### Biological Activity of Targeted CMP Trimeric 4-1BB Ligand-Containing Antigen Binding Molecules

5.1. NF-κB-Luciferase Activation in Transgenic Reporter Cell Line HeLa Cells Expressing Human 4-1BB

5.1.1. Generation of HeLa Cells Expressing Human 4-1BB and NF-κB-Luciferase

The cervix carcinoma cell line HeLa (ATCC CCL-2) was transduced with a plasmid based on the expression vector pETR10829, which contains the sequence of human 4-1BB (Uniprot accession Q07011) under control of a CMV-promoter and a puromycin resistance gene. Cells were cultured in DMEM medium supplemented with 10% (v/v) FBS, 1% (v/v) GlutaMAX-I and 3 μg/mL Puromycin.

4-1BB-transduced HeLa cells were tested for 4-1BB expression by flow cytometry: 0.2×10⁶ living cells were resuspended in 100 μL FACS buffer containing 0.1 μg PerCP/Cy5.5 conjugated anti-human 4-1BB mouse IgG1κ clone 4B4-1 (BioLegend Cat.-No. 309814) or its isotype control (PerCP/Cy5.5 conjugated mouse IgG1κ isotype control antibody clone MOPC-21, BioLegend Cat.-No. 400150) and incubated for 30 minutes at 4° C. Cells were washed twice with FACS buffer, resuspended in 300 μL FACS buffer containing 0.06 μg DAPI (Santa Cruz Biotec, Cat. No. Sc-3598) and acquired using a 5-laser LSR-Fortessa (BD Bioscience, DIVA software). Limited dilutions were performed to generate single clones as described: human-4-1BB-transduced HeLa cells were resuspended in medium to a density of 10, 5 and 2.5 cells/ml and 200 μl of cell suspensions were transferred to round bottom tissue-culture treated 96-well plates (6 plates/cell concentration, TPP Cat.-No. 92697). Single clones were harvested, expanded and tested for 4-1BB expression as described above. The clone with the highest expression of 4-1BB (clone 5) was chosen for subsequent transfection with the NFκB-luciferase expression-vector 5495p Tranlucent HygB. The vector confers transfected cells both with resistance to Hygromycin B and capacity to express luciferase under control of NF-κB-response element (Panomics, Cat.-No. LR0051). Human-4-1BB HeLa clone 5 cells were cultured to 70% confluence. 50 μg (40 μL) linearized (restriction enzymes AseI and SalI) 5495p Tranlucent HygB expression vector were added to a sterile 0.4 cm Gene Pulser/MicroPulser Cuvette (Biorad, Cat.-No, 165-2081). 2.5×10⁶ human-4-1BB HeLa clone 5 cells in 400 μl supplement-free DMEM medium were added and mixed carefully with the plasmid solution. Transfection of cells was performed using a Gene Pulser Xcell total system (Biorad, Cat-No. 165-2660) under the following settings: exponential pulse, capacitance 500 μF, voltage 160 V, resistance ∞. Immediately after the pulse transfected cells were transferred to a 75 cm² tissue culture flask (TPP, Cat.-No. 90075) with 15 mL 37° C. warm DMEM medium supplied with 10% (v/v) FBS and 1% (v/v) GlutaMAX-I. Next day, culture medium containing 3 μg/mL Puromycin and 200 μg/mL Hygromycin B (Roche, Cat.-No. 10843555001) was added. Surviving cells were expanded and limited dilution was performed as described above to generate single clones.

Clones were tested for 4-1BB expression as described above and for NF-κB-Luciferase activity as following: Clones were harvested in selection medium and counted using a Cell Counter Vi-cell xr 2.03 (Beckman Coulter, Cat.-No. 731050). Cells were set to a cell density of 0.33×10⁶ cells/mL and 150 μL of this cell suspension were transferred to each well of a sterile white 96-well flat bottom tissue culture plate with lid (greiner bio-one, Cat.-No. 655083) and—as a control—to normal 96-well flat bottom tissue culture plate (TPP Cat.-No. 92096) to test survival and cell density the next day. Cells were incubated at 37° C. and 5% CO₂ overnight. The next day 50 μL of medium containing different concentrations of recombinant human tumor necrosis factor alpha (rhTNF-α, PeproTech, Cat.-No. 300-01A) were added to each well of a 96-well plate resulting in final concentration of rhTNF-α of 100, 50, 25, 12.5, 6.25 and 0 ng/well. Cells were incubated for 6 hours at 37° C. and 5% CO₂ and then washed three times with 200 μL/well DPBS. Reporter Lysis Buffer (Promega, Cat-No: E3971) was added to each well (40 μl) and the plates were stored over night at −20° C. The next day frozen cell plates and Detection Buffer (Luciferase 1000 Assay System, Promega, Cat.-No. E4550) were thawed to room temperature. 100 uL of detection buffer were added to each well and the plate was measured as fast as possible using a SpectraMax M5/M5e microplate reader and the SoftMax Pro Software (Molecular Devices). Measured units of released light for 500 ms/well (URLs) above control (no rhTNF-α added) were taken as luciferase activity. The NF-κB-luc-4-1BB-HeLa clone 26 exhibiting the highest luciferase activity and a considerable level of 4-1BB-expression and was chosen for further use.

5.1.2. NF-κB Activation in Hela Cells Expressing Human 4-1BB Co-Cultured with FAP-Expressing Tumor Cells

NF-κB-luciferase human-4-1BB HeLa cells were harvested and resuspended in DMEM medium supplied with 10% (v/v) FBS and 1% (v/v) GlutaMAX-I to a concentration of 0.2×10⁶ cells/ml. 100 μl (2×10⁴ cells) of this cell suspension were transferred to each well of a sterile white 96-well flat bottom tissue culture plate with lid (greiner bio-one, Cat. No. 655083) and the plate were incubated at 37° C. and 5% CO₂ overnight. The next day 50 μL of medium containing different concentrations of titrated FAP-targeted CMP-trimeric 4-1BB ligand-containing molecule or DP47-untargeted CMP-trimeric 4-1BB ligand-containing molecule were added. FAP-expressing tumor cells were resuspended in DMEM medium supplied with 10% (v/v) FBS and 1% (v/v) GlutaMAX-I to a concentration of 2×10⁶ cells/ml.

The following FAP-expressing tumor cells were used:
human melanoma cell line MV3 (first published: van Muijen G N, Jansen K F, Cornelissen I M, Smeets D F, Beck J L, Ruiter D J. Establishment and characterization of a human melanoma cell line (MV3) which is highly metastatic in nude mice. Int J Cancer. 1991 Apr. 22; 48(1):85-91)
human female melanoma WM-266-4 cell line (ATCC CRL-1676).
mouse embryonic fibroblast NIH/3T3 (ATCC CRL-1658) transfected with expression vector pETR4921 to express human FAP and Puromycin resistance: NIH/3T3-huFAP clone 39 as described in Example 4.2.

50 μL of FAP-expressing tumor cell suspension were added to each well and plates were incubated for 6 hours at 37° C. and 5% CO₂. Cells were washed three times with 200 μL/well DPBS. 40 μl freshly prepared Reporter Lysis Buffer (Promega, Cat-No: E3971) were added to each well and the plate were stored over night at −20° C. The next day frozen cell plate and Detection Buffer (Luciferase 1000 Assay System, Promega, Cat. No. E4550) were thawed at room temperature. 100 μL of detection buffer were added to each well and luciferase activity was measured as fast as possible using a SpectraMax M5/M5e microplate reader and a SoftMax Pro Software (Molecular Devices).

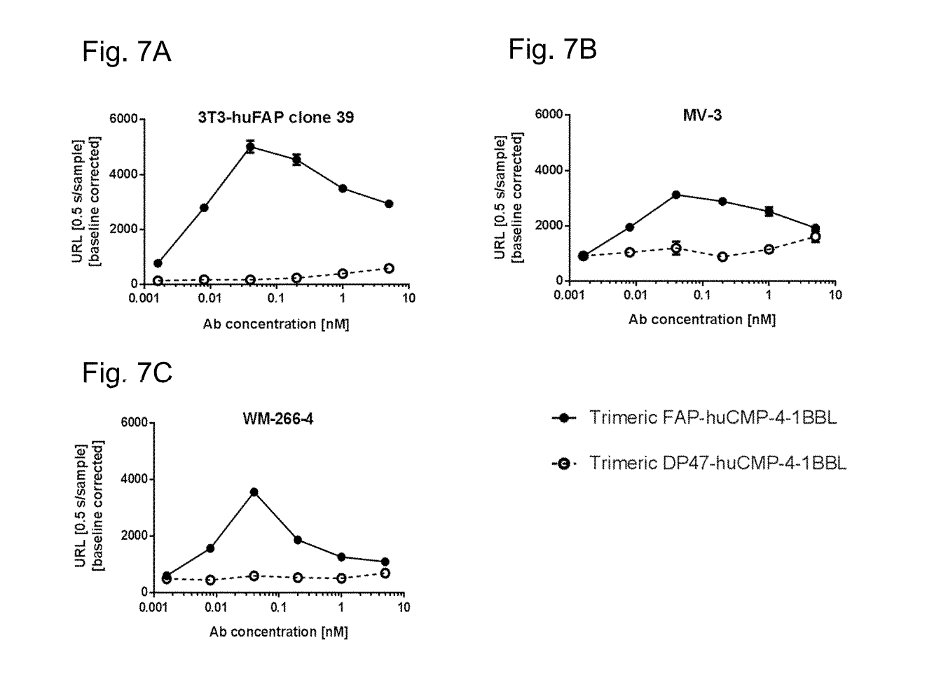

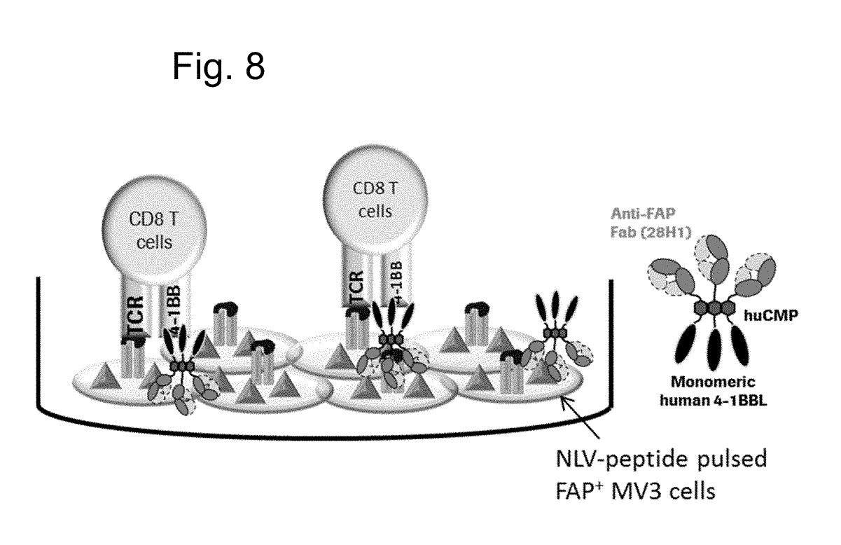

As shown in FIG. 7, FAP-targeted huCMP-trimeric 4-1BB ligand-containing antigen binding molecule (filled circles) triggered activation of the NF-κB signaling pathway in the reporter cell line in the presence of FAP-expressing tumor cells. In contrast, the untargeted variant of the same molecule failed to trigger such an effect at any of the tested concentrations (open circles). This activity of targeted 4-1BBL was strictly dependent on the expression of FAP at the cell surface of tumor cells as no NFkB activation could be detected upon culturing of the NF-κB reporter cell line with FAP-negative tumor cells even in the presence of FAP-targeted huCMP-trimeric 4-1BB ligand-containing antigen binding molecule (data not shown). Further activation depends on an optimal concentration window supporting a maximal crosslinking of the FAP-targeted huCMP-trimeric 4-1BB ligand-containing antigen binding molecule (bell-shape curves). The maximal activation is induced at a concentration around 0.3-0.4 nM.

5.1.3. Antigen-Specific CD8+ T Cell Based Assay

Cells of the human FAP-expres sing melanoma cell line MV3 cell line (described in Example 5.1.2.) were harvested and washed with DPBS and 2×10⁷ cells were resuspended in 250 μL C diluent of the PKH-26 Red Fluorescence Cell linker Kit (Sigma, Cat.-No. PKH26GL). 1 μL PKH26-red-stain solution was diluted with 250 μL C diluent and added to the suspension of MV3 cells which were then incubated for 5 min at room temperature in the dark. 0.5 mL FBS were added, cells were incubated for 1 minute and washed once with T cell medium consisting of RPMI 1640 medium supplemented with 10% (v/v) FBS, 1% (v/v) GlutaMAX-I, 1 mM Sodium-Pyruvate, 1% (v/v) MEM non-essential amino acids and 50 μM β-Mercaptoethanol. 1×10⁶ MV3 cells/mL were resuspended in T cell medium and separated into three tubes. Synthetic NLVPMVATV peptide (obtained from think peptides) was added to a final concentration of 1×10⁻⁹ M or 1×10⁻⁸ M and cells were incubated for 90 min under rotation at 37° C. and 5% CO₂. MV3 cells were washed once with T cell medium and resuspended to a density of 0.5×10⁶ cells/mL, distributed (100 μL/well) to a 96-well round bottom cell-suspension plate (Greiner bio-one, cellstar, Cat. No. 650185) and incubated over night at 37° C. and 5% CO₂.

The next day, different concentrations of FAP-targeted or DP47-untargeted huCMP-trimeric 4-1BB ligand-containing antigen binding molecules were added with 50 μL of T-cell medium (final concentrations of FAP-targeted or DP47-targeted single chain trimeric 4-1BB ligand were between 5 to 0.002 nM). NLV-specific CD8 T cells were harvested, CFSE-labeled and added in 50 μL medium to each well (final tumor: CD8 T cell ratio 0.125). Cells were incubated for 24 h, 50 μL/well T cell medium containing 2.64 μL/mL Golgi stop (Protein Transport Inhibitor containing Monesin, BD Bioscience, Cat.-No. 554724) were added to each well. Cells were incubated for further 4 h, washed with 200 μL/well DPBS and stained with 100 μL 4° C. DPBS containing 1:5000 diluted Fixable Viability Dye-eF450 (eBioscience, Cat. No. 65-0864) for 30 minutes at 4° C. Cells were washed with DPBS and the stained in 40 μL/well FACS buffer containing following fluorescent dye-conjugated antibodies: anti-human CD137-PerCP/Cy5.5 (clone 4B4-1, mouse IgG1κ, BioLegend, Cat.-No. 309814), anti-human CD70-PE (clone 113-16, mouse IgG1κ, BioLegend, Cat.-No. 355104) and anti-human PD-1-PE/Cy7 (clone EH12.2H7, mouse IgG1κ, BioLegend, Cat.-No. 329918). After incubation for 30 min at 4° C., cells were washed twice with 200 μL/well FACS buffer, resuspended in 50 μL/well freshly prepared FoxP3 Fix/Perm buffer (eBioscience Cat.-No. 00-5123 and 00-5223) and incubated for 30 min at 4° C. Plates were washed twice with 200 μL/well Perm-Buffer (DPBS supplied with 2% (v/v) FBS, 1% (w/v) saponin (Sigma Life Science, S7900) and 1% (w/v) sodium azide (Sigma-Aldrich, S2002) and stained with 50 μL/well Perm-Buffer (eBioscience, Cat.-No. 00-8333-56) containing 0.25 μg/mL anti-human IFNγ-APC (clone B27, mouse IgG1κ, BioLegend, Cat. No. 506510). Plates were incubated for 1 h at 4° C. and washed twice with 200 μL/well Perm-Buffer. For fixation, 50 μL/well DPBS containing 1% formaldehyde were added and cells were stored overnight at 4° C. The next day, cells were resuspended in 100 μL/well FACS buffer and acquired using a 5-laser Fortessa flow cytometer (BD Bioscience with DIVA software).

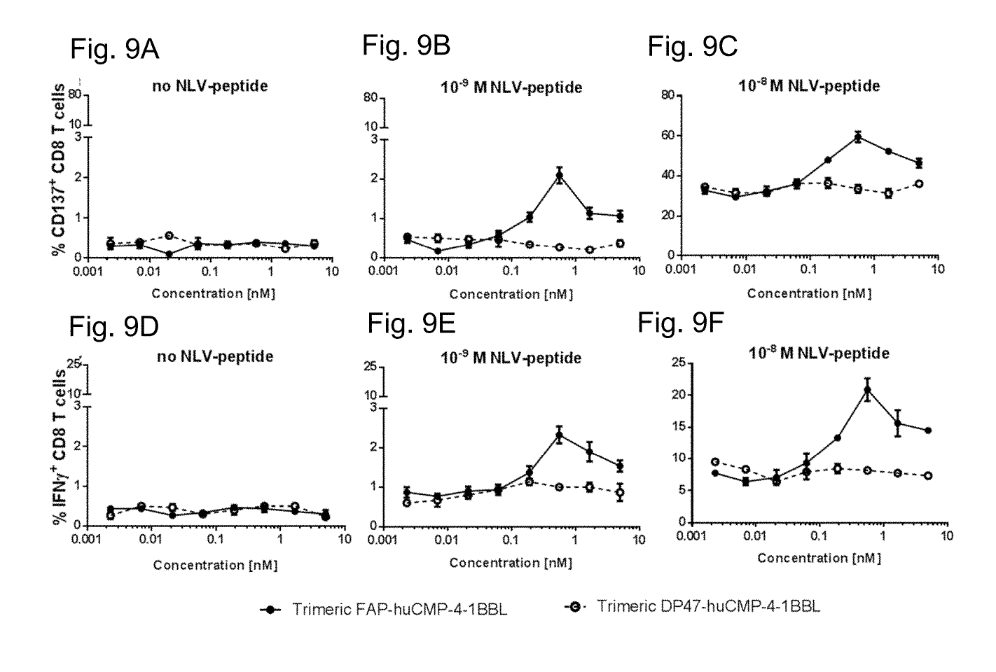

As shown in FIG. 9, antigen-specific CD8⁺ T cells, but not unstimulated controls, exhibited increased levels of surface 4-1BB expression in the presence of FAP-targeted huCMP trimeric 4-1BB ligand-containing antigen binding molecules (filled circles). This effect of FAP-targeted huCMP trimeric 4-1BB ligand-containing antigen binding molecules was dose dependent and required FAP-targeting as addition of the untargeted control molecule did not affect the level of 4-1BB expression. Furthermore, T-cells activated with peptide showed sustained secretion of INFγ in the presence of FAP-targeted huCMP trimeric 4-1BB ligand-containing antigen binding molecules. Collectively, these data demonstrate that the FAP-targeted huCMP trimeric 4-1BB ligand-containing antigen binding molecule modulates the surface phenotype and responsiveness of antigen specific T-cells in a targeting dependent manner.

### Example 6

### Functional Characterization of Targeted huCMP Trimeric OX40 Ligand-Containing Antigen Binding Molecules

6.1. Binding on Human FAP Positive Tumor Cells

The binding to cell surface FAP was tested using WM-266-4 cells (ATCC CRL-1676). 5×10⁴ WM-266-4 cells were added to each well of a round-bottom suspension cell 96-well plates (greiner bio-one, cellstar, Cat. No. 650185). Cells were stained for 120 minutes at 4° C. in the dark in 50 μL/well 4° C. cold FACS buffer (DPBS (Gibco by Life Technologies, Cat. No. 14190 326) w/ BSA (0.1% v/w, Sigma-Aldrich, Cat. No. A9418) containing titrated huCMP trimeric OX40 ligand-containing antigen binding molecules. After three times washing with excess FACS buffer, cells were stained for 60 minutes at 4° C. in the dark in 25 μL/well 4° C. cold FACS buffer containing Fluorescein isothiocyanate (FITC)-conjugated AffiniPure anti-human IgG F(ab′)₂-fragment-specific goat IgG F(ab′)₂ fragment (Jackson ImmunoResearch, Cat.-No. 109-096-097).

Plates were finally resuspended in 90 μL/well FACS-buffer containing 0.2 μg/mL DAPI (Santa Cruz Biotec, Cat. No. Sc-3598) and acquired the same day using 5-laser LSR-Fortessa (BD Bioscience with DIVA software).

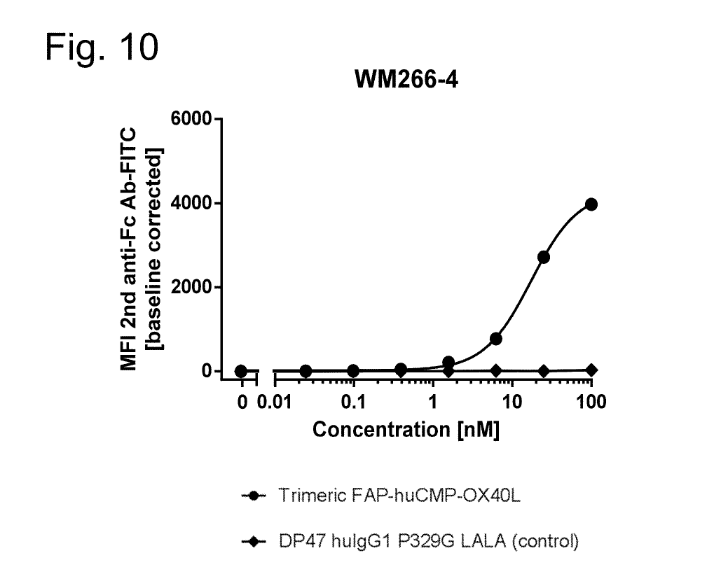

As shown in FIG. 10, the FAP(28H1)-targeted huCMP trimeric Ox40 ligand-containing antigen binding molecule (trimeric FAP-huCMP-OX40L) but not the negative control (an unspecific DP47 hu IgG1 P329G LALA antibody termed control F) efficiently bound to human FAP-expressing target cells. EC₅₀ value of binding to FAP positive WM-266-4 was 17.3 nM.

6.2. Binding to Human Ox40 Expressing Cells: Naïve and Activated Human Peripheral Mononuclear Blood Leukocytes (PBMC)

Buffy coats were obtained from the Zurich blood donation center. To isolate fresh peripheral blood mononuclear cells (PBMCs) the buffy coat was diluted with the same volume of DPBS (Gibco by Life Technologies, Cat. No. 14190 326). 50 mL polypropylene centrifuge tubes (TPP, Cat.-No. 91050) were supplied with 15 mL Histopaque 1077 (SIGMA Life Science, Cat.-No. 10771, polysucrose and sodium diatrizoate, adjusted to a density of 1.077 g/mL) and the buffy coat solution was layered above the Histopaque 1077. The tubes were centrifuged for 30 min at 400×g, room temperature and with low acceleration and no break. Afterwards the PBMCs were collected from the interface, washed three times with DPBS and resuspended in T cell medium consisting of RPMI 1640 medium (Gibco by Life Technology, Cat. No. 42401-042) supplied with 10% Fetal Bovine Serum (FBS, Gibco by Life Technology, Cat. No. 16000-044, Lot 941273, gamma-irradiated, mycoplasma-free and heat inactivated at 56° C. for 35 min), 1% (v/v) GlutaMAX I (GIBCO by Life Technologies, Cat. No. 35050 038), 1 mM Sodium-Pyruvat (SIGMA, Cat. No. S8636), 1% (v/v) MEM non-essential amino acids (SIGMA, Cat.-No. M7145) and 50 μM β-Mercaptoethanol (SIGMA, M3148).

PBMCs were used directly after isolation (binding on resting human PBMCs) or they were stimulated to receive a strong human Ox40 expression on the cell surface of T cells (binding on activated human PBMCs). Therefore naïve PBMCs were cultured for three days in T cell medium supplied with 200 U/mL Proleukin (Novartis) and 2 mg/mL PHA-L (Sigma-Aldrich, L2769-10) in 6-well tissue culture plate at 37° C. and 5% CO₂.

Cells were stained for 120 minutes at 4° C. in the dark in 50 μL/well 4° C. cold FACS buffer containing titrated anti-Ox40 antibody constructs. After three times washing with excess FACS buffer, cells were stained for 1 h at 4° C. in the dark in 25 μL/well 4° C. cold FACS buffer containing a mixture of fluorescently labeled anti-human CD4 (clone OKT4, mouse IgG1 k, BioLegend, Cat.-No. 317428), anti-human CD8 (clone RPa-T8, mouse IgG1k, BD Pharmingen, Cat.-No. 555368) and Fluorescein isothiocyanate (FITC)-conjugated AffiniPure anti-human IgG F(ab′)2-fragment-specific goat IgG F(ab′)₂ fragment (Jackson ImmunoResearch, Cat.-No. 109-096-097). Plates were finally resuspended in 90 μL/well FACS-buffer containing 0.2 μg/mL DAPI (Santa Cruz Biotec, Cat. No. Sc-3598) and acquired the same day using 5-laser LSR-Fortessa (BD Bioscience with DIVA software).

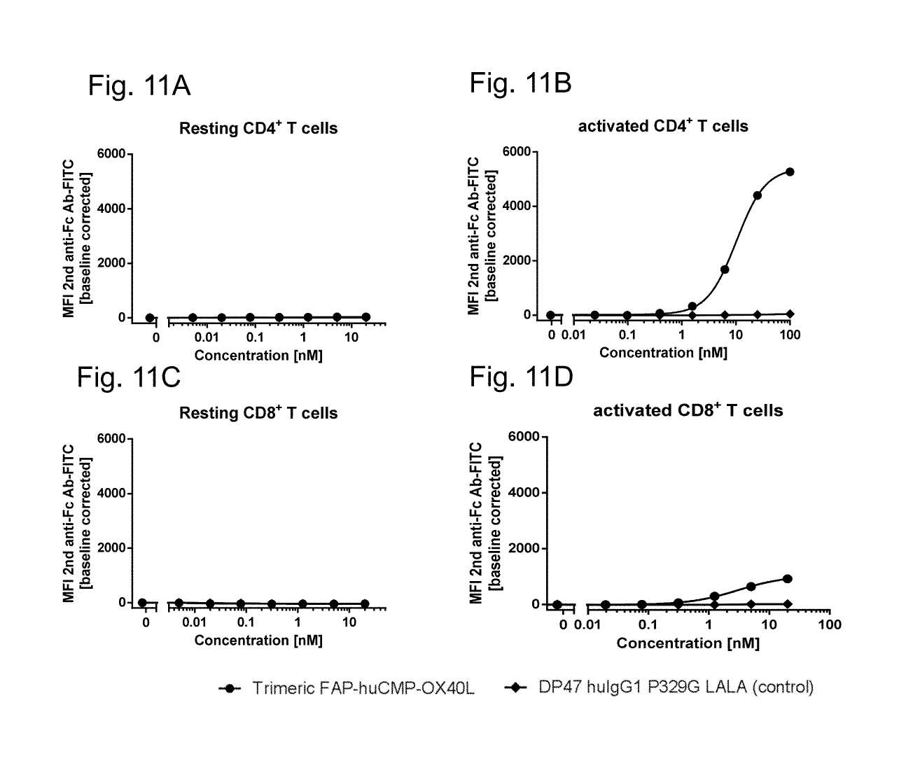

As shown in FIGS. 11A to 11D, FAP-targeted huCMP trimeric OX40 ligand-containing antigen binding molecules did not bind to resting human CD4⁺ T-cells or CD8⁺ T-cells, which are negative for OX40. In contrast, FAP-targeted huCMP trimeric Ox40 ligand-containing antigen binding molecules bound to activated CD8⁺ or CD4⁺ T-cells, which do express OX40. Binding to CD4⁺ T-cells was much stronger than that to CD8⁺ T cells. Activated human CD8⁺ T cells do express only a fraction of the OX40 levels detected on activated CD4⁺ T cells. Expression levels for Ox40 are depending on kinetic and strength of stimulation and conditions were here optimized for Ox40 expression on CD4⁺ T cells but not for CD8⁺ T cells. Thus, only little OX40 expression was induced on CD8 T cells. The EC₅₀ value of binding to OX40 positive CD4⁺ T cells was 10.2 nM and the EC₅₀ value of binding to OX40 positive CD8⁺ T cells was 3.0 nM.

6.3. HeLa Cells Expressing Human OX40 and Reporter Gene NF-κB-Luciferase

Agonstic binding of OX40 to its ligand induces downstream signaling via activation of nuclear factor kappa B (NFκB) (A. D. Weinberg et al., J. Leukoc. Biol. 2004, 75(6), 962-972). The recombinant reporter cell line HeLa_hOx40_NFkB_Luc1 was generated to express human OX40 on its surface. Additionally, it harbors a reporter plasmid containing the luciferase gene under the control of an NFκB-sensitive enhancer segment. OX40 triggering induces dose-dependent activation of NFκB, which translocates in the nucleus, where it binds on the NFκB sensitive enhancer of the reporter plasmid to increase expression of the luciferase protein. Luciferase catalyzes luciferin-oxidation resulting in oxyluciferin which emits light. This can be quantified by a luminometer. Thus, the capacity of the various anti-OX40 molecules to induce NFκB activation in HeLa_hOx40_NFkB_Luc1 reporter cells was analyzed as a measure for bioactivity.

Adherent HeLa_hOx40_NFkB_Luc1 cells were harvested using cell dissociation buffer (Invitrogen, Cat.-No. 13151-014) for 10 minutes at 37° C. Cells were washed once with DPBS and were adjusted to a cell density of 2×10⁵ in assay media comprising of MEM (Invitrogen, Cat.-No. 22561-021), 10% (v/v) heat-inactivated FBS, 1 mM Sodium-Pyruvat and 1% (v/v) non-essential amino acids. Cells were seeded in a density of 0.3*10⁵ cells per well in a sterile white 96-well flat bottom tissue culture plate with lid (greiner bio-one, Cat. No. 655083) and kept over night at 37° C. and 5% CO₂ in an incubator (Hera Cell 150).

The next day, HeLa_hOx40_NFkB_Luc1 were stimulated for 6 hours by adding assay medium containing titrated FAP-Ox40L or negative control F. For testing the effect of hyper-crosslinking on anti-Ox40 antibodies, 25 μL/well of medium containing FAP+ tumor cell line NIH/3T3-huFAP clone 39 was added in a 1:2 ratio (2 times more NIH/3T3-huFAP clone 39 than HeLa_hOx40_NFkB_Luc1 cells).

NIH/3T3-huFAP clone 39 was generated by the transfection of the mouse embryonic fibroblast NIH/3T3 cell line (ATCC CRL-1658) with the expression vector pETR4921 to express huFAP under 1.5 μg/mL Puromycin selection. The surface expression of FAP was quantified using the Quifikit (Dako Cat. No. K0078) according to manufactures instructions. The primary antibody used to detect cell surface FAP expression was the human/mouse crossreactive clone F11-24 (mouse IgG1, Calbiochem, Ca. No. OP188). The surface expression on NIH/3T3-huFAP clone 39 was app. 90000 huFAP per cell.

After incubation, supernatant was aspirated and plates washed two times with DPBS. Quantification of light emission was done using the luciferase 100 assay system and the reporter lysis buffer (both Promega, Cat.-No. E4550 and Cat-No: E3971) according to manufacturer instructions. Briefly, cells were lysed for 10 minutes at −20° C. by addition of 30 uL per well 1× lysis buffer. Cells were thawed for 20 minutes at 37° C. before 90 uL per well provided luciferase assay reagent was added. Light emission was quantified immediately with a SpectraMax M5/M5e microplate reader (Molecular Devices, USA) using 500 ms integration time, without any filter to collect all wavelengths. Emitted relative light units (URL) were corrected by basal luminescence of HeLa_hOx40_NFkB_Luc1 cells and were blotted against the logarithmic primary antibody concentration using Prism4 (GraphPad Software, USA). Curves were fitted using the inbuilt sigmoidal dose response.

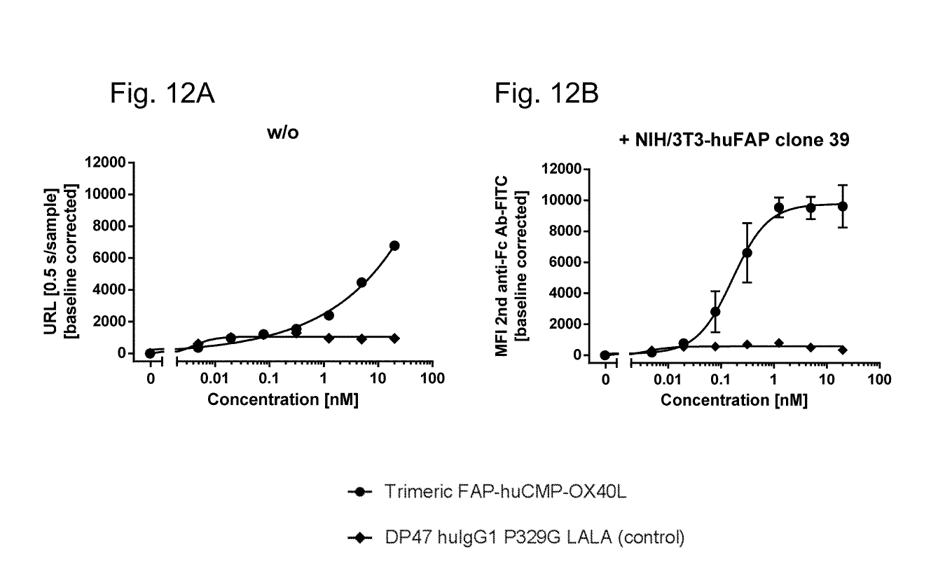

As shown in FIGS. 12A and 12B, limited, dose dependent NFκB activation was induced already by addition of FAP-targeted huCMP trimeric OX40 ligand-containing antigen binding molecules to the reporter cell line (FIG. 12A). The presence of FAP-expressing tumor cells strongly increased induction of NFκB-mediated luciferase-activation when FAP-targeted huCMP trimeric OX40 ligand-containing antigen binding molecule was added (FIG. 12B).

### CITATIONS

Aggarwal B. B. (2003), Signalling pathways of the TNF superfamily: a double-edged sword. Nat. Rev. Immunol. 3(9), 745-56.
Banner D. et al (1993), Crystal structure of the soluble human 55 kd TNF receptor-human TNF beta complex: implications for TNF receptor activation. Cell 73, 431-445.
Beck K., Gambee J., Bohan C., Bächinger H. P. (1996), The C-terminal domain of cartilage matrix protein assembles into a triple-stranded α-helical coiled-coil structure. J. Mol. Biol. 256, 909-923.
Bodmer J., Schneider P. and Tschopp, J. (2002), The molecular architecture of the TNF superfamily. Trends in Biochemical Sciences 27(1), 19-26.
Broll, K., Richter, G., Pauly, S., Hofstaedter, F., and Schwarz, H. (2001). CD137 expression in tumor vessel walls. High correlation with malignant tumors. Am J Clin Pathol 115, 543-549.
Buechele, C., Baessler, T., Schmiedel, B. J., Schumacher, C. E., Grosse-Hovest, L., Rittig, K., and Salih, H. R. (2012). 4-1BB ligand modulates direct and Rituximab-induced NK-cell reactivity in chronic lymphocytic leukemia. Eur J Immunol 42, 737-748.
Choi, B. K., Kim, Y. H., Kwon, P. M., Lee, S. C., Kang, S. W., Kim, M. S., Lee, M. J., and Kwon, B. S. (2009). 4-1BB functions as a survival factor in dendritic cells. J Immunol 182, 4107-4115.
Cuadros, C., Dominguez, A. L., Lollini, P. L., Croft, M., Mittler, R. S., Borgstrom, P., and Lustgarten, J. (2005). Vaccination with dendritic cells pulsed with apoptotic tumors in combination with anti-OX40 and anti-4-1BB monoclonal antibodies induces T cell-mediated protective immunity in Her-2/neu transgenic mice. Int J Cancer 116, 934-943.
Curran, M. A., Kim, M., Montalvo, W., Al-Shamkhani, A., and Allison, J. P. (2011). Combination CTLA-4 blockade and 4-1BB activation enhances tumor rejection by increasing T-cell infiltration, proliferation, and cytokine production. PLoS One 6, e19499.
Diehl, L., van Mierlo, G. J., den Boer, A. T., van der Voort, E., Fransen, M., van Bostelen, L., Krimpenfort, P., Melief, C. J., Mittler, R., Toes, R. E., and Offringa, R. (2002). In vivo triggering through 4-1BB enables Th-independent priming of CTL in the presence of an intact CD28 costimulatory pathway. J Immunol 168, 3755-3762.
Dubrot, J., Milheiro, F., Alfaro, C., Palazon, A., Martinez-Forero, I., Perez-Gracia, J. L., Morales-Kastresana, A., Romero-Trevejo, J. L., Ochoa, M. C., Hervas-Stubbs, S., et al. (2010). Treatment with anti-CD137 mAbs causes intense accumulations of liver T cells without selective antitumor immunotherapeutic effects in this organ. Cancer Immunol Immunother 59, 1223-1233.
Futagawa, T., Akiba, H., Kodama, T., Takeda, K., Hosoda, Y., Yagita, H., and Okumura, K. (2002). Expression and function of 4-1BB and 4-1BB ligand on murine dendritic cells. Int Immunol 14, 275-286.
Guo, Z., Cheng, D., Xia, Z., Luan, M., Wu, L., Wang, G., and Zhang, S. (2013). Combined TIM-3 blockade and CD137 activation affords the long-term protection in a murine model of ovarian cancer. J Transl Med 11, 215.
Heinisch, I. V., Daigle, I., Knopfli, B., and Simon, H. U. (2000). CD137 activation abrogates granulocyte-macrophage colony-stimulating factor-mediated anti-apoptosis in neutrophils. Eur J Immunol 30, 3441-3446.
Ju, S. A., Cheon, S. H., Park, S. M., Tam, N. Q., Kim, Y. M., An, W. G., and Kim, B. S. (2008). Eradication of established renal cell carcinoma by a combination of 5-fluorouracil and anti-4-1BB monoclonal antibody in mice. Int J Cancer 122, 2784-2790.
Kienzle, G., and von Kempis, J. (2000). CD137 (ILA/4-1BB), expressed by primary human monocytes, induces monocyte activation and apoptosis of B lymphocytes. Int Immunol 12, 73-82.
Kim, D. H., Chang, W. S., Lee, Y. S., Lee, K. A., Kim, Y. K., Kwon, B. S., and Kang, C. Y. (2008). 4-1BB engagement costimulates NKT cell activation and exacerbates NKT cell ligand-induced airway hyperresponsiveness and inflammation. J Immunol 180, 2062-2068.
Kim, Y. H., Choi, B. K., Oh, H. S., Kang, W. J., Mittler, R. S., and Kwon, B. S. (2009). Mechanisms involved in synergistic anticancer effects of anti-4-1BB and cyclophosphamide therapy. Mol Cancer Ther 8, 469-478.
Kwon, B. S., and Weissman, S. M. (1989). cDNA sequences of two inducible T-cell genes. Proc Natl Acad Sci USA 86, 1963-1967.
Lee, H., Park, H. J., Sohn, H. J., Kim, J. M., and Kim, S. J. (2011). Combinatorial therapy for liver metastatic colon cancer: dendritic cell vaccine and low-dose agonistic anti-4-1BB antibody co-stimulatory signal. J Surg Res 169, e43-50.
Li, F., and Ravetch, J. V. (2011). Inhibitory Fcgamma receptor engagement drives adjuvant and anti-tumor activities of agonistic CD40 antibodies. Science 333, 1030-1034.
Lin, W., Voskens, C. J., Zhang, X., Schindler, D. G., Wood, A., Burch, E., Wei, Y., Chen, L., Tian, G., Tamada, K., et al. (2008). Fc-dependent expression of CD137 on human NK cells:
insights into “agonistic” effects of anti-CD137 monoclonal antibodies. Blood 112, 699-707.
Melero, I., Johnston, J. V., Shufford, W. W., Mittler, R. S., and Chen, L. (1998). NK1.1 cells express 4-1BB (CDw137) costimulatory molecule and are required for tumor immunity elicited by anti-4-1BB monoclonal antibodies. Cell Immunol 190, 167-172.
Melero, I., Shuford, W. W., Newby, S. A., Aruffo, A., Ledbetter, J. A., Hellstrom, K. E., Mittler, R. S., and Chen, L. (1997). Monoclonal antibodies against the 4-1BB T-cell activation molecule eradicate established tumors. Nat Med 3, 682-685.
Merchant, A. M., Zhu, Z., Yuan, J. Q., Goddard, A., Adams, C. W., Presta, L. G., and Carter, P. (1998). An efficient route to human bispecific IgG. Nat Biotechnol 16, 677-681.
Morales-Kastresana, A., Sanmamed, M. F., Rodriguez, I., Palazon, A., Martinez-Forero, I., Labiano, S., Hervas-Stubbs, S., Sangro, B., Ochoa, C., Rouzaut, A., et al. (2013). Combined immunostimulatory monoclonal antibodies extend survival in an aggressive transgenic hepatocellular carcinoma mouse model. Clin Cancer Res 19, 6151-6162.
Murillo, O., Dubrot, J., Palazon, A., Arina, A., Azpilikueta, A., Alfaro, C., Solano, S., Ochoa, M. C., Berasain, C., Gabari, I., et al. (2009). In vivo depletion of DC impairs the anti-tumor effect of agonistic anti-CD137 mAb. Eur J Immunol 39, 2424-2436.
Narazaki, H., Zhu, Y., Luo, L., Zhu, G., and Chen, L. (2010). CD137 agonist antibody prevents cancer recurrence: contribution of CD137 on both hematopoietic and nonhematopoietic cells. Blood 115, 1941-1948.
Nishimoto, H., Lee, S. W., Hong, H., Potter, K. G., Maeda-Yamamoto, M., Kinoshita, T., Kawakami, Y., Mittler, R. S., Kwon, B. S., Ware, C. F., et al. (2005). Costimulation of mast cells by 4-1BB, a member of the tumor necrosis factor receptor superfamily, with the high-affinity IgE receptor. Blood 106, 4241-4248.
Olofsson, P. S., Soderstrom, L. A., Wagsater, D., Sheikine, Y., Ocaya, P., Lang, F., Rabu, C., Chen, L., Rudling, M., Aukrust, P., et al. (2008). CD137 is expressed in human atherosclerosis and promotes development of plaque inflammation in hypercholesterolemic mice. Circulation 117, 1292-1301.
Palazon, A., Teijeira, A., Martinez-Forero, I., Hervas-Stubbs, S., Roncal, C., Penuelas, I., Dubrot, J., Morales-Kastresana, A., Perez-Gracia, J. L., Ochoa, M. C., et al. (2011). Agonist anti-CD137 mAb act on tumor endothelial cells to enhance recruitment of activated T lymphocytes. Cancer Res 71, 801-811.
Schwarz, H., Valbracht, J., Tuckwell, J., von Kempis, J., and Lotz, M. (1995). ILA, the human 4-1BB homologue, is inducible in lymphoid and other cell lineages. Blood 85, 1043-1052.
Shao, Z., and Schwarz, H. (2011). CD137 ligand, a member of the tumor necrosis factor family, regulates immune responses via reverse signal transduction. J Leukoc Biol 89, 21-29.
Shi, W., and Siemann, D. W. (2006). Augmented antitumor effects of radiation therapy by 4-1BB antibody (BMS-469492) treatment. Anticancer Res 26, 3445-3453.
Simeone, E., and Ascierto, P. A. (2012). Immunomodulating antibodies in the treatment of metastatic melanoma: the experience with anti-CTLA-4, anti-CD137, and anti-PD1. J Immunotoxicol 9, 241-247.
Snell, L. M., Lin, G. H., McPherson, A. J., Moraes, T. J., and Watts, T. H. (2011). T-cell intrinsic effects of GITR and 4-1BB during viral infection and cancer immunotherapy. Immunol Rev 244, 197-217.
Stagg, J., Loi, S., Divisekera, U., Ngiow, S. F., Duret, H., Yagita, H., Teng, M. W., and Smyth, M. J. (2011). Anti-ErbB-2 mAb therapy requires type I and II interferons and synergizes with anti-PD-1 or anti-CD137 mAb therapy. Proc Natl Acad Sci USA 108, 7142-7147.
Teng, M. W., Sharkey, J., McLaughlin, N. M., Exley, M. A., and Smyth, M. J. (2009). CD1d-based combination therapy eradicates established tumors in mice. J Immunol 183, 1911-1920.
von Kempis, J., Schwarz, H., and Lotz, M. (1997). Differentiation-dependent and stimulus-specific expression of ILA, the human 4-1BB-homologue, in cells of mesenchymal origin. Osteoarthritis Cartilage 5, 394-406.
Watts, T. H. (2005). TNF/TNFR family members in costimulation of T cell responses. Annu. Rev. Immunol. 23, 23-68
Wei, H., Zhao, L., Li, W., Fan, K., Qian, W., Hou, S., Wang, H., Dai, M., Hellstrom, I., Hellstrom, K. E., and Guo, Y. (2013). Combinatorial PD-1 blockade and CD137 activation has therapeutic efficacy in murine cancer models and synergizes with cisplatin. PLoS One 8, e84927.
Weinberg A. D., Evans D. E., Thalhofer C, Shi T., and Prell R. A. (2004). The generation of T cell memory: a review describing the molecular and cellular events following OX40 (CD134) engagement. J. Leukoc. Biol. 75, 962-972.
Wilcox, R. A., Chapoval, A. I., Gorski, K. S., Otsuji, M., Shin, T., Flies, D. B., Tamada, K., Mittler, R. S., Tsuchiya, H., Pardoll, D. M., and Chen, L. (2002). Cutting edge: Expression of functional CD137 receptor by dendritic cells. J Immunol 168, 4262-4267.
Wilcox, R. A., Tamada, K., Flies, D. B., Zhu, G., Chapoval, A. I., Blazar, B. R., Kast, W. M., and Chen, L. (2004). Ligation of CD137 receptor prevents and reverses established anergy of CD8+ cytolytic T lymphocytes in vivo. Blood 103, 177-184.
Wyzgol A., Müller N., Fick A., Munkel S., Grigoleit G. U., Pfizenmaier K., Wajant H. (2009), Trimer Stabilization, Oligomerization, and Antibody-mediated cell surface immobilization improve the activity of soluble trimers of CD27L, CD40L, 41BBL and Glucocorticoid-induced TNF receptor ligand. J. Immunol. 183, 1851-1861.
Zhang, X., Voskens, C. J., Sallin, M., Maniar, A., Montes, C. L., Zhang, Y., Lin, W., Li, G., Burch, E., Tan, M., et al. (2010). CD137 promotes proliferation and survival of human B cells. J Immunol 184, 787-795.

1. A trimeric costimulatory TNF family ligand-containing antigen binding molecule of claim 1 comprising three fusion polypeptides, each of the three fusion polypeptides comprising
(a) an ectodomain of a costimulatory TNF family ligand selected from the group consisting of 4-1BBL, OX40L and GITRL or fragments thereof,
(b) a trimerization domain derived from human cartilage matrix protein (huCMP) of amino acid sequence of SEQ ID NO:1, and
(c) a moiety capable of specific binding to a target cell antigen.

2. The trimeric costimulatory TNF family ligand-containing antigen binding molecule of claim 1, wherein the trimerization domain comprises an amino acid sequence having at least 95% identity to SEQ ID NO:2.

3. The trimeric costimulatory TNF family ligand-containing antigen binding molecule of claim 1, wherein the trimerization domain comprises the amino acid sequence of SEQ ID NO:2.

4. The trimeric costimulatory TNF family ligand-containing antigen binding molecule of claims 1 to 3, wherein the costimulatory TNF family ligand is selected from 4-1BBL and OX40L.

5. The trimeric costimulatory TNF family ligand-containing antigen binding molecule of claims 1 to 4, wherein the costimulatory TNF family ligand is 4-1BBL

6. The trimeric costimulatory TNF family ligand-containing antigen binding molecule of any one of claims 1 to 5, wherein the ectodomain of a TNF family ligand comprises the amino acid sequence selected from the group consisting of SEQ ID NO:3, SEQ ID NO:4, SEQ ID NO:5, SEQ ID NO:6, SEQ ID NO:7, SEQ ID NO:8, SEQ ID NO:9 and SEQ ID NO:10, particularly the amino acid sequence of SEQ ID NO:3 or SEQ ID NO:7.

7. The trimeric costimulatory TNF family ligand-containing antigen binding molecule of any one of claims 1 to 6, comprising three fusion polypeptides, each of the three fusion polypeptides comprising
(a) an ectodomain of a TNF family ligand comprising the amino acid sequence selected from the group consisting of SEQ ID NO:3, SEQ ID NO:4, SEQ ID NO:5, SEQ ID NO:6, SEQ ID NO:7, SEQ ID NO:8, SEQ ID NO:9 and SEQ ID NO:10,
(b) a trimerization domain comprising the amino acid sequence of SEQ ID NO:2 and
(c) a moiety capable of specific binding to a target cell antigen.

8. The trimeric costimulatory TNF family ligand-containing antigen binding molecule as defined herein before, wherein the costimulatory TNF family ligand is OX40L.

9. The trimeric costimulatory TNF family ligand-containing antigen binding molecule of any one of claim 1 to 4 or 8, wherein the ectodomain of a TNF family ligand comprises the amino acid sequence of SEQ ID NO:11, SEQ ID NO:12 or SEQ ID NO:13.

10. The trimeric costimulatory TNF family ligand-containing antigen binding molecule of any one of claim 1 to 4 or 8 or 9, comprising three fusion polypeptides, wherein each of the three fusion polypeptides comprises
(a) an ectodomain of a TNF family ligand comprising the amino acid sequence of SEQ ID NO:11, SEQ ID NO:12 or SEQ ID NO:13,
(b) a trimerization domain comprising the amino acid sequence of SEQ ID NO:2 and
(c) a moiety capable of specific binding to a target cell antigen.

11. The trimeric costimulatory TNF family ligand-containing antigen binding molecule of any one of claims 1 to 10, wherein the ectodomain of a TNF ligand family member or a fragment thereof is fused at the N-terminal amino acid to the C-terminal amino acid of the trimerization domain, optionally through a peptide linker.

12. The trimeric costimulatory TNF family ligand-containing antigen binding molecule of any one of claims 1 to 11, wherein the moiety capable of specific binding to a target cell antigen is fused at the C-terminal amino acid to the N-terminal amino acid of the trimerization domain, optionally through a peptide linker.

13. The trimeric costimulatory TNF family ligand-containing antigen binding molecule of any one of claims 1 to 12, wherein the moiety capable of specific binding to a target cell antigen is a Fab molecule capable of specific binding to a target cell antigen.

14. The trimeric costimulatory TNF family ligand-containing antigen binding molecule of claim 13, wherein said Fab molecule is fused at the C-terminal amino acid of the CH1 domain to the N-terminal amino acid of the trimerization domain, optionally through a peptide linker.

15. The trimeric costimulatory TNF family ligand-containing antigen binding molecule of any one of claims 1 to 14, wherein the target cell antigen is selected from the group consisting of Fibroblast Activation Protein (FAP), Melanoma-associated Chondroitin Sulfate Proteoglycan (MCSP), Epidermal Growth Factor Receptor (EGFR), Carcinoembryonic Antigen (CEA), CD19, CD20 and CD33.

16. The trimeric costimulatory TNF family ligand-containing antigen binding molecule of any one of claims 1 to 13, wherein the target cell antigen is Fibroblast Activation Protein (FAP).

17. The trimeric costimulatory TNF family ligand-containing antigen binding molecule of any one of claims 1 to 16, wherein the moiety capable of specific binding to FAP comprises
(a) a VH domain comprising (i) CDR-H1 comprising the amino acid sequence of SEQ ID NO:14, (ii) CDR-H2 comprising the amino acid sequence of SEQ ID NO:15 and (iii) CDR-H3 comprising the amino acid sequence of SEQ ID NO:16, and a VL domain comprising (iv) CDR-L1 comprising the amino acid sequence of SEQ ID NO:17, (v) CDR-L2 comprising the amino acid sequence of SEQ ID NO:18 and (vi) CDR-L3 comprising the amino acid sequence of SEQ ID NO:19 or
(b) a VH domain comprising (i) CDR-H1 comprising the amino acid sequence of SEQ ID NO:20, (ii) CDR-H2 comprising the amino acid sequence of SEQ ID NO:21 and (iii) CDR-H3 comprising the amino acid sequence of SEQ ID NO:22, and a VL domain comprising (iv) CDR-L1 comprising the amino acid sequence of SEQ ID NO:23, (v) CDR-L2 comprising the amino acid sequence of SEQ ID NO:24 and (vi) CDR-L3 comprising the amino acid sequence of SEQ ID NO:25.

18. A fusion polypeptide comprising (a) an ectodomain of a costimulatory TNF family ligand selected from the group consisting of 4-1BBL, OX40L and GITRL or a fragment thereof, (b) a trimerization domain derived from human cartilage matrix protein (huCMP) comprising the amino acid sequence of SEQ ID NO:2, wherein said trimerization domain is capable of mediating stable association of said fusion polypeptide with two further such fusion polypeptides and (c) a moiety capable of specific binding to a target cell antigen.

19. The fusion polypeptide of claim 18, wherein the fusion polypeptide comprises (c) a VH or a VL domain capable of specific binding to a target cell antigen.

20. The fusion polypeptide of claim 18 or 19, wherein the fusion polypeptide comprises
(a) an ectodomain of a TNF family ligand comprising the amino acid sequence selected from the group consisting of SEQ ID NO:3, SEQ ID NO:4, SEQ ID NO:5, SEQ ID NO:6, SEQ ID NO:7, SEQ ID NO:8, SEQ ID NO:9 and SEQ ID NO:10,
(b) a trimerization domain comprising the amino acid sequence of SEQ ID NO:2 and
(c) a VH domain comprising (i) CDR-H1 comprising the amino acid sequence of SEQ ID NO:14, (ii) CDR-H2 comprising the amino acid sequence of SEQ ID NO:15 and (iii) CDR-H3 comprising the amino acid sequence of SEQ ID NO:16 or a VH domain comprising (i) CDR-H1 comprising the amino acid sequence of SEQ ID NO:20, (ii) CDR-H2 comprising the amino acid sequence of SEQ ID NO:21 and (iii) CDR-H3 comprising the amino acid sequence of SEQ ID NO:22.

21. The fusion polypeptide of claim 18 or 19, wherein the fusion polypeptide comprises
(a) an ectodomain of a TNF family ligand comprising the amino acid sequence selected from the group consisting of SEQ ID NO:11, SEQ ID NO:12 and SEQ ID NO:13,
(b) a trimerization domain comprising the amino acid sequence of SEQ ID NO:2 and
(c) a VH domain comprising (i) CDR-H1 comprising the amino acid sequence of SEQ ID NO:14, (ii) CDR-H2 comprising the amino acid sequence of SEQ ID NO:15 and (iii) CDR-H3 comprising the amino acid sequence of SEQ ID NO:16 or a VH domain comprising (i) CDR-H1 comprising the amino acid sequence of SEQ ID NO:20, (ii) CDR-H2 comprising the amino acid sequence of SEQ ID NO:21 and (iii) CDR-H3 comprising the amino acid sequence of SEQ ID NO:22.

22. A polynucleotide encoding the trimeric costimulatory TNF family ligand-containing antigen binding molecule of claims 1 to 17 or the fusion polypeptide of claims 18 to 21.

23. An expression vector comprising the polynucleotide of claim 22.

24. A host cell comprising the polynucleotide of claim 22 or the expression vector of claim 23.

25. A method of producing a trimeric costimulatory TNF family ligand-containing antigen binding molecule, comprising culturing the host cell of claim 24 under conditions suitable for the expression of said trimeric antigen binding molecule and isolating said trimeric antigen binding molecule.

26. A pharmaceutical composition comprising the trimeric costimulatory TNF family ligand-containing antigen binding molecule of claims 1 to 17 and at least one pharmaceutically acceptable excipient.

27. The trimeric costimulatory TNF family ligand-containing antigen binding molecule of claims 1 to 17, or the pharmaceutical composition of claim 26, for use as a medicament.

28. The trimeric costimulatory TNF family ligand-containing antigen binding molecule of claims 1 to 17, or the pharmaceutical composition of claim 26, for use in the treatment of cancer.

29. Use of the trimeric costimulatory TNF family ligand-containing antigen binding molecule of claims 1 to 17, or the pharmaceutical composition of claim 26, in the manufacture of a medicament for the treatment of cancer.

30. A method of treating an individual having cancer comprising administering to the individual an effective amount of the trimeric costimulatory TNF family ligand-containing antigen binding molecule of claims 1 to 17, or the pharmaceutical composition of claim 26.


SEQUENCE LISTING (93 sequences)

SEQ ID NO: 1 - protein, 496 aa, human
MRVLSGTSLMLCSLLLLLQALCSPGLAPQSRGHLCRTRPTDLVFVVDSSRSVRPVEFEKVKVFLSQVIESLDVGPNATRVGMVNYASTVKQEFSLRAHVSKAALLQAVRRIQPLSTGTMTGLAIQFAITKAFGDAEGGRSRSPDISKVVIVVTDGRPQDSVQDVSARARASGVELFAIGVGSVDKATLRQIASEPQDEHVDYVESYSVIEKLSRKFQEAFCVVSDLCATGDHDCEQVCISSPGSYTCACHEGFTLNSDGKTCNVCSGGGGSSATDLVFLIDGSKSVRPENFELVKKFISQIVDTLDVSDKLAQVGLVQYSSSVRQEFPLGRFHTKKDIKAAVRNMSYMEKGTMTGAALKYLIDNSFTVSSGARPGAQKVGIVFTDGRSQDYINDAAKKAKDLGFKMFAVGVGNAVEDELREIASEPVAEHYFYTADFKTINQIGKKLQKKICVEEDPCACESLVKFQAKVEGLLQALTRKLEAVSKRLAILENTVV

SEQ ID NO: 2 - protein, 39 aa, human
CACESLVKFQAKVEGLLQALTRKLEAVSKRLAILENTVV

SEQ ID NO: 3 - protein, 184 aa, human
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 4 - protein, 170 aa, human
LDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 5 - protein, 175 aa, human
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 6 - protein, 203 aa, human
PWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 7 - protein, 178 aa, human
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 8 - protein, 164 aa, human
LDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 9 - protein, 169 aa, humand
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 10 - protein, 197 aa, human
PWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 11 - protein, 133 aa, human
QVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 12 - protein, 133 aa, Artificial
  Ox40L with mutations N90D, N114D
QVSHRYPRIQSIKVQFTEYKKEKGFILTSQKEDEIMKVQDNSVIINCDGFYLISLKGYFSQEVDISLHYQKDEEPLFQLKKVRSVNSLMVASLTYKDKVYLNVTTDNTSLDDFHVNGGELILIHQNPGEFCVL

SEQ ID NO: 13 - protein, 132 aa, human
VSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 14 - protein, 5 aa, Artificial Sequence
  FAP(28H1)  CDR-H1
SHAMS

SEQ ID NO: 15 - protein, 16 aa, Artificial Sequence
  FAP(28H1)  CDR-H2
AIWASGEQYYADSVKG

SEQ ID NO: 16 - protein, 8 aa, Artificial Sequence
  FAP(28H1)  CDR-H3
GWLGNFDY

SEQ ID NO: 17 - protein, 12 aa, Artificial Sequence
  FAP(28H1)  CDR-L1
RASQSVSRSYLA

SEQ ID NO: 18 - protein, 7 aa, Artificial Sequence
  FAP(28H1)  CDR-L2
GASTRAT

SEQ ID NO: 19 - protein, 9 aa, Artificial Sequence
  FAP(28H1)  CDR-L3
QQGQVIPPT

SEQ ID NO: 20 - protein, 5 aa, Artificial Sequence
  FAP(4B9)  CDR-H1
SYAMS

SEQ ID NO: 21 - protein, 17 aa, Artificial Sequence
  FAP(4B9)  CDR-H2
AIIGSGASTYYADSVKG

SEQ ID NO: 22 - protein, 8 aa, Artificial Sequence
  FAP(4B9)  CDR-H3
GWFGGFNY

SEQ ID NO: 23 - protein, 12 aa, Artificial Sequence
  FAP(4B9)  CDR-L1
RASQSVSRSYLA

SEQ ID NO: 24 - protein, 7 aa, Artificial Sequence
  FAP(4B9)  CDR-L2
VGSRRAT

SEQ ID NO: 25 - protein, 9 aa, Artificial Sequence
  FAP(4B9)  CDR-L3
QQGIMLPPT

SEQ ID NO: 26 - protein, 116 aa, Artificial Sequence
  FAP(28H1) VH
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSS

SEQ ID NO: 27 - protein, 108 aa, Artificial Sequence
  FAP(28H1) VL
EIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIK

SEQ ID NO: 28 - protein, 117 aa, Artificial Sequence
  FAP(4B9)  VH
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 29 - protein, 108 aa, Artificial Sequence
  FAP(4B9)  VL
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 30 - protein, 760 aa, human
MKTWVKIVFGVATSAVLALLVMCIVLRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 31 - protein, 748 aa, Artificial Sequence
  hu FAP ectodomain+poly-lys-tag+his6-tag
RPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSDGKKKKKKGHHHHHH

SEQ ID NO: 32 - DNA, 2244 bp, Artificial Sequence
  nucleotide sequence hu FAP ectodomain+poly-lys-tag+his6-tag
cgcccttcaagagttcataactctgaagaaaatacaatgagagcactcacactgaaggatattttaaatggaacattttcttataaaacattttttccaaactggatttcaggacaagaatatcttcatcaatctgcagataacaatatagtactttataatattgaaacaggacaatcatataccattttgagtaatagaaccatgaaaagtgtgaatgcttcaaattacggcttatcacctgatcggcaatttgtatatctagaaagtgattattcaaagctttggagatactcttacacagcaacatattacatctatgaccttagcaatggagaatttgtaagaggaaatgagcttcctcgtccaattcagtatttatgctggtcgcctgttgggagtaaattagcatatgtctatcaaaacaatatctatttgaaacaaagaccaggagatccaccttttcaaataacatttaatggaagagaaaataaaatatttaatggaatcccagactgggtttatgaagaggaaatgcttgctacaaaatatgctctctggtggtctcctaatggaaaatttttggcatatgcggaatttaatgatacggatataccagttattgcctattcctattatggcgatgaacaatatcctagaacaataaatattccatacccaaaggctggagctaagaatcccgttgttcggatatttattatcgataccacttaccctgcgtatgtaggtccccaggaagtgcctgttccagcaatgatagcctcaagtgattattatttcagttggctcacgtgggttactgatgaacgagtatgtttgcagtggctaaaaagagtccagaatgtttcggtcctgtctatatgtgacttcagggaagactggcagacatgggattgtccaaagacccaggagcatatagaagaaagcagaactggatgggctggtggattctttgtttcaacaccagttttcagctatgatgccatttcgtactacaaaatatttagtgacaaggatggctacaaacatattcactatatcaaagacactgtggaaaatgctattcaaattacaagtggcaagtgggaggccataaatatattcagagtaacacaggattcactgttttattctagcaatgaatttgaagaataccctggaagaagaaacatctacagaattagcattggaagctatcctccaagcaagaagtgtgttacttgccatctaaggaaagaaaggtgccaatattacacagcaagtttcagcgactacgccaagtactatgcacttgtctgctacggcccaggcatccccatttccacccttcatgatggacgcactgatcaagaaattaaaatcctggaagaaaacaaggaattggaaaatgctttgaaaaatatccagctgcctaaagaggaaattaagaaacttgaagtagatgaaattactttatggtacaagatgattcttcctcctcaatttgacagatcaaagaagtatcccttgctaattcaagtgtatggtggtccctgcagtcagagtgtaaggtctgtatttgctgttaattggatatcttatcttgcaagtaaggaagggatggtcattgccttggtggatggtcgaggaacagctttccaaggtgacaaactcctctatgcagtgtatcgaaagctgggtgtttatgaagttgaagaccagattacagctgtcagaaaattcatagaaatgggtttcattgatgaaaaaagaatagccatatggggctggtcctatggaggatacgtttcatcactggcccttgcatctggaactggtcttttcaaatgtggtatagcagtggctccagtctccagctgggaatattacgcgtctgtctacacagagagattcatgggtctcccaacaaaggatgataatcttgagcactataagaattcaactgtgatggcaagagcagaatatttcagaaatgtagactatcttctcatccacggaacagcagatgataatgtgcactttcaaaactcagcacagattgctaaagctctggttaatgcacaagtggatttccaggcaatgtggtactctgaccagaaccacggcttatccggcctgtccacgaaccacttatacacccacatgacccacttcctaaagcagtgtttctctttgtcagacggcaaaaagaaaaagaaaaagggccaccaccatcaccatcac

SEQ ID NO: 33 - protein, 761 aa, murine
MKTWLKTVFGVTTLAALALVVICIVLRPSRVYKPEGNTKRALTLKDILNGTFSYKTYFPNWISEQEYLHQSEDDNIVFYNIETRESYIILSNSTMKSVNATDYGLSPDRQFVYLESDYSKLWRYSYTATYYIYDLQNGEFVRGYELPRPIQYLCWSPVGSKLAYVYQNNIYLKQRPGDPPFQITYTGRENRIFNGIPDWVYEEEMLATKYALWWSPDGKFLAYVEFNDSDIPIIAYSYYGDGQYPRTINIPYPKAGAKNPVVRVFIVDTTYPHHVGPMEVPVPEMIASSDYYFSWLTWVSSERVCLQWLKRVQNVSVLSICDFREDWHAWECPKNQEHVEESRTGWAGGFFVSTPAFSQDATSYYKIFSDKDGYKHIHYIKDTVENAIQITSGKWEAIYIFRVTQDSLFYSSNEFEGYPGRRNIYRISIGNSPPSKKCVTCHLRKERCQYYTASFSYKAKYYALVCYGPGLPISTLHDGRTDQEIQVLEENKELENSLRNIQLPKVEIKKLKDGGLTFWYKMILPPQFDRSKKYPLLIQVYGGPCSQSVKSVFAVNWITYLASKEGIVIALVDGRGTAFQGDKFLHAVYRKLGVYEVEDQLTAVRKFIEMGFIDEERIAIWGWSYGGYVSSLALASGTGLFKCGIAVAPVSSWEYYASIYSERFMGLPTKDDNLEHYKNSTVMARAEYFRNVDYLLIHGTADDNVHFQNSAQIAKALVNAQVDFQAMWYSDQNHGISSGRSQNHLYTHMTHFLKQCFSLSD

SEQ ID NO: 34 - protein, 749 aa, Artificial Sequence
  Murine FAP ectodomain+poly-lys-tag+his6-tag
RPSRVYKPEGNTKRALTLKDILNGTFSYKTYFPNWISEQEYLHQSEDDNIVFYNIETRESYIILSNSTMKSVNATDYGLSPDRQFVYLESDYSKLWRYSYTATYYIYDLQNGEFVRGYELPRPIQYLCWSPVGSKLAYVYQNNIYLKQRPGDPPFQITYTGRENRIFNGIPDWVYEEEMLATKYALWWSPDGKFLAYVEFNDSDIPIIAYSYYGDGQYPRTINIPYPKAGAKNPVVRVFIVDTTYPHHVGPMEVPVPEMIASSDYYFSWLTWVSSERVCLQWLKRVQNVSVLSICDFREDWHAWECPKNQEHVEESRTGWAGGFFVSTPAFSQDATSYYKIFSDKDGYKHIHYIKDTVENAIQITSGKWEAIYIFRVTQDSLFYSSNEFEGYPGRRNIYRISIGNSPPSKKCVTCHLRKERCQYYTASFSYKAKYYALVCYGPGLPISTLHDGRTDQEIQVLEENKELENSLRNIQLPKVEIKKLKDGGLTFWYKMILPPQFDRSKKYPLLIQVYGGPCSQSVKSVFAVNWITYLASKEGIVIALVDGRGTAFQGDKFLHAVYRKLGVYEVEDQLTAVRKFIEMGFIDEERIAIWGWSYGGYVSSLALASGTGLFKCGIAVAPVSSWEYYASIYSERFMGLPTKDDNLEHYKNSTVMARAEYFRNVDYLLIHGTADDNVHFQNSAQIAKALVNAQVDFQAMWYSDQNHGILSGRSQNHLYTHMTHFLKQCFSLSDGKKKKKKGHHHHHH

SEQ ID NO: 35 - DNA, 2247 bp, Artificial Sequence
  nucleotide sequence of murine FAP ectodomain+poly-lys-tag+his6-tag
cgtccctcaagagtttacaaacctgaaggaaacacaaagagagctcttaccttgaaggatattttaaatggaacattctcatataaaacatattttcccaactggatttcagaacaagaatatcttcatcaatctgaggatgataacatagtattttataatattgaaacaagagaatcatatatcattttgagtaatagcaccatgaaaagtgtgaatgctacagattatggtttgtcacctgatcggcaatttgtgtatctagaaagtgattattcaaagctctggcgatattcatacacagcgacatactacatctacgaccttcagaatggggaatttgtaagaggatacgagctccctcgtccaattcagtatctatgctggtcgcctgttgggagtaaattagcatatgtatatcaaaacaatatttatttgaaacaaagaccaggagatccaccttttcaaataacttatactggaagagaaaatagaatatttaatggaataccagactgggtttatgaagaggaaatgcttgccacaaaatatgctctttggtggtctccagatggaaaatttttggcatatgtagaatttaatgattcagatataccaattattgcctattcttattatggtgatggacagtatcctagaactataaatattccatatccaaaggctggggctaagaatccggttgttcgtgtttttattgttgacaccacctaccctcaccacgtgggcccaatggaagtgccagttccagaaatgatagcctcaagtgactattatttcagctggctcacatgggtgtccagtgaacgagtatgcttgcagtggctaaaaagagtgcagaatgtctcagtcctgtctatatgtgatttcagggaagactggcatgcatgggaatgtccaaagaaccaggagcatgtagaagaaagcagaacaggatgggctggtggattctttgtttcgacaccagcttttagccaggatgccacttcttactacaaaatatttagcgacaaggatggttacaaacatattcactacatcaaagacactgtggaaaatgctattcaaattacaagtggcaagtgggaggccatatatatattccgcgtaacacaggattcactgttttattctagcaatgaatttgaaggttaccctggaagaagaaacatctacagaattagcattggaaactctcctccgagcaagaagtgtgttacttgccatctaaggaaagaaaggtgccaatattacacagcaagtttcagctacaaagccaagtactatgcactcgtctgctatggccctggcctccccatttccaccctccatgatggccgcacagaccaagaaatacaagtattagaagaaaacaaagaactggaaaattctctgagaaatatccagctgcctaaagtggagattaagaagctcaaagacgggggactgactttctggtacaagatgattctgcctcctcagtttgacagatcaaagaagtaccctttgctaattcaagtgtatggtggtccttgtagccagagtgttaagtctgtgtttgctgttaattggataacttatctcgcaagtaaggaggggatagtcattgccctggtagatggtcggggcactgctttccaaggtgacaaattcctgcatgccgtgtatcgaaaactgggtgtatatgaagttgaggaccagctcacagctgtcagaaaattcatagaaatgggtttcattgatgaagaaagaatagccatatggggctggtcctacggaggttatgtttcatccctggcccttgcatctggaactggtcttttcaaatgtggcatagcagtggctccagtctccagctgggaatattacgcatctatctactcagagagattcatgggcctcccaacaaaggacgacaatctcgaacactataaaaattcaactgtgatggcaagagcagaatatttcagaaatgtagactatcttctcatccacggaacagcagatgataatgtgcactttcagaactcagcacagattgctaaagctttggttaatgcacaagtggatttccaggcgatgtggtactctgaccagaaccatggtatattatctgggcgctcccagaatcatttatatacccacatgacgcacttcctcaagcaatgcttttctttatcagacggcaaaaagaaaaagaaaaagggccaccaccatcaccatcac

SEQ ID NO: 36 - protein, 748 aa, Artificial Sequence
  Cynomolgus FAP ectodomain+poly-lys-tag+his6-tag
RPPRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPFVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEDYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSDGKKKKKKGHHHHHH

SEQ ID NO: 37 - DNA, 2244 bp, Artificial Sequence
  nucleotide sequence of cynomolgus FAP ectodomain+poly-lys-tag+his6-tag
cgccctccaagagttcataactctgaagaaaatacaatgagagcactcacactgaaggatattttaaatgggacattttcttataaaacattttttccaaactggatttcaggacaagaatatcttcatcaatctgcagataacaatatagtactttataatattgaaacaggacaatcatataccattttgagtaacagaaccatgaaaagtgtgaatgcttcaaattatggcttatcacctgatcggcaatttgtatatctagaaagtgattattcaaagctttggagatactcttacacagcaacatattacatctatgaccttagcaatggagaatttgtaagaggaaatgagcttcctcgtccaattcagtatttatgctggtcgcctgttgggagtaaattagcatatgtctatcaaaacaatatctatttgaaacaaagaccaggagatccaccttttcaaataacatttaatggaagagaaaataaaatatttaatggaatcccagactgggtttatgaagaggaaatgcttgctacaaaatatgctctctggtggtctcctaatggaaaatttttggcatatgcggaatttaatgatacagatataccagttattgcctattcctattatggcgatgaacaatatcccagaacaataaatattccatacccaaaggccggagctaagaatccttttgttcggatatttattatcgataccacttaccctgcgtatgtaggtccccaggaagtgcctgttccagcaatgatagcctcaagtgattattatttcagttggctcacgtgggttactgatgaacgagtatgtttgcagtggctaaaaagagtccagaatgtttcggtcttgtctatatgtgatttcagggaagactggcagacatgggattgtccaaagacccaggagcatatagaagaaagcagaactggatgggctggtggattctttgtttcaacaccagttttcagctatgatgccatttcatactacaaaatatttagtgacaaggatggctacaaacatattcactatatcaaagacactgtggaaaatgctattcaaattacaagtggcaagtgggaggccataaatatattcagagtaacacaggattcactgttttattctagcaatgaatttgaagattaccctggaagaagaaacatctacagaattagcattggaagctatcctccaagcaagaagtgtgttacttgccatctaaggaaagaaaggtgccaatattacacagcaagtttcagcgactacgccaagtactatgcacttgtctgctatggcccaggcatccccatttccacccttcatgacggacgcactgatcaagaaattaaaatcctggaagaaaacaaggaattggaaaatgctttgaaaaatatccagctgcctaaagaggaaattaagaaacttgaagtagatgaaattactttatggtacaagatgattcttcctcctcaatttgacagatcaaagaagtatcccttgctaattcaagtgtatggtggtccctgcagtcagagtgtaaggtctgtatttgctgttaattggatatcttatcttgcaagtaaggaagggatggtcattgccttggtggatggtcggggaacagctttccaaggtgacaaactcctgtatgcagtgtatcgaaagctgggtgtttatgaagttgaagaccagattacagctgtcagaaaattcatagaaatgggtttcattgatgaaaaaagaatagccatatggggctggtcctatggaggatatgtttcatcactggcccttgcatctggaactggtcttttcaaatgtgggatagcagtggctccagtctccagctgggaatattacgcgtctgtctacacagagagattcatgggtctcccaacaaaggatgataatcttgagcactataagaattcaactgtgatggcaagagcagaatatttcagaaatgtagactatcttctcatccacggaacagcagatgataatgtgcactttcaaaactcagcacagattgctaaagctctggttaatgcacaagtggatttccaggcaatgtggtactctgaccagaaccacggcttatccggcctgtccacgaaccacttatacacccacatgacccacttcctaaagcagtgtttctctttgtcagacggcaaaaagaaaaagaaaaagggccaccaccatcaccatcac

SEQ ID NO: 38 - protein, 702 aa, human
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNKLSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 39 - protein, 2322 aa, human
MQSGPRPPLPAPGLALALTLTMLARLASAASFFGENHLEVPVATALTDIDLQLQFSTSQPEALLLLAAGPADHLLLQLYSGRLQVRLVLGQEELRLQTPAETLLSDSIPHTVVLTVVEGWATLSVDGFLNASSAVPGAPLEVPYGLFVGGTGTLGLPYLRGTSRPLRGCLHAATLNGRSLLRPLTPDVHEGCAEEFSASDDVALGFSGPHSLAAFPAWGTQDEGTLEFTLTTQSRQAPLAFQAGGRRGDFIYVDIFEGHLRAVVEKGQGTVLLHNSVPVADGQPHEVSVHINAHRLEISVDQYPTHTSNRGVLSYLEPRGSLLLGGLDAEASRHLQEHRLGLTPEATNASLLGCMEDLSVNGQRRGLREALLTRNMAAGCRLEEEEYEDDAYGHYEAFSTLAPEAWPAMELPEPCVPEPGLPPVFANFTQLLTISPLVVAEGGTAWLEWRHVQPTLDLMEAELRKSQVLFSVTRGARHGELELDIPGAQARKMFTLLDVVNRKARFIHDGSEDTSDQLVLEVSVTARVPMPSCLRRGQTYLLPIQVNPVNDPPHIIFPHGSLMVILEHTQKPLGPEVFQAYDPDSACEGLTFQVLGTSSGLPVERRDQPGEPATEFSCRELEAGSLVYVHRGGPAQDLTFRVSDGLQASPPATLKVVAIRPAIQIHRSTGLRLAQGSAMPILPANLSVETNAVGQDVSVLFRVTGALQFGELQKQGAGGVEGAEWWATQAFHQRDVEQGRVRYLSTDPQHHAYDTVENLALEVQVGQEILSNLSFPVTIQRATVWMLRLEPLHTQNTQQETLTTAHLEATLEEAGPSPPTFHYEVVQAPRKGNLQLQGTRLSDGQGFTQDDIQAGRVTYGATARASEAVEDTFRFRVTAPPYFSPLYTFPIHIGGDPDAPVLTNVLLVVPEGGEGVLSADHLFVKSLNSASYLYEVMERPRHGRLAWRGTQDKTTMVTSFTNEDLLRGRLVYQHDDSETTEDDIPFVATRQGESSGDMAWEEVRGVFRVAIQPVNDHAPVQTISRIFHVARGGRRLLTTDDVAFSDADSGFADAQLVLTRKDLLFGSIVAVDEPTRPIYRFTQEDLRKRRVLFVHSGADRGWIQLQVSDGQHQATALLEVQASEPYLRVANGSSLVVPQGGQGTIDTAVLHLDTNLDIRSGDEVHYHVTAGPRWGQLVRAGQPATAFSQQDLLDGAVLYSHNGSLSPRDTMAFSVEAGPVHTDATLQVTIALEGPLAPLKLVRHKKIYVFQGEAAEIRRDQLEAAQEAVPPADIVFSVKSPPSAGYLVMVSRGALADEPPSLDPVQSFSQEAVDTGRVLYLHSRPEAWSDAFSLDVASGLGAPLEGVLVELEVLPAAIPLEAQNFSVPEGGSLTLAPPLLRVSGPYFPTLLGLSLQVLEPPQHGALQKEDGPQARTLSAFSWRMVEEQLIRYVHDGSETLTDSFVLMANASEMDRQSHPVAFTVTVLPVNDQPPILTTNTGLQMWEGATAPIPAEALRSTDGDSGSEDLVYTIEQPSNGRVVLRGAPGTEVRSFTQAQLDGGLVLFSHRGTLDGGFRFRLSDGEHTSPGHFFRVTAQKQVLLSLKGSQTLTVCPGSVQPLSSQTLRASSSAGTDPQLLLYRVVRGPQLGRLFHAQQDSTGEALVNFTQAEVYAGNILYEHEMPPEPFWEAHDTLELQLSSPPARDVAATLAVAVSFEAACPQRPSHLWKNKGLWVPEGQRARITVAALDASNLLASVPSPQRSEHDVLFQVTQFPSRGQLLVSEEPLHAGQPHFLQSQLAAGQLVYAHGGGGTQQDGFHFRAHLQGPAGASVAGPQTSEAFAITVRDVNERPPQPQASVPLRLTRGSRAPISRAQLSVVDPDSAPGEIEYEVQRAPHNGFLSLVGGGLGPVTRFTQADVDSGRLAFVANGSSVAGIFQLSMSDGASPPLPMSLAVDILPSAIEVQLRAPLEVPQALGRSSLSQQQLRVVSDREEPEAAYRLIQGPQYGHLLVGGRPTSAFSQFQIDQGEVVFAFTNFSSSHDHFRVLALARGVNASAVVNVTVRALLHVWAGGPWPQGATLRLDPTVLDAGELANRTGSVPRFRLLEGPRHGRVVRVPRARTEPGGSQLVEQFTQQDLEDGRLGLEVGRPEGRAPGPAGDSLTLELWAQGVPPAVASLDFATEPYNAARPYSVALLSVPEAARTEAGKPESSTPTGEPGPMASSPEPAVAKGGFLSFLEANMFSVIIPMCLVLLLLALILPLLFYLRKRNKTGKHDVQVLTAKPRNGLAGDTETFRKVEPGQAIPLTAVPGQGPPPGGQPDPELLQFCRTPNPALKNGQYWV

SEQ ID NO: 40 - protein, 1210 aa, human [sequence omitted]

SEQ ID NO: 41 - protein, 556 aa, human [sequence omitted]

SEQ ID NO: 42 - protein, 297 aa, human
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 43 - protein, 364 aa, human
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 44 - protein, 183 aa, human
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 45 - protein, 193 aa, human
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 46 - protein, 234 aa, human
MDPGLQQALNGMAPPGDTAMHVPAGSVASHLGTTSRSYFYLTTATLALCLVFTVATIMVLVVQRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 47 - protein, 254 aa, human
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 48 - protein, 240 aa, human
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 49 - protein, 199 aa, human
MTLHPSPITCEFLFSTALISPKMCLSHLENMPLSHSRTQGAQRSSWKLWLFCSIVMLLFLCSFSWLIFIFLQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 50 - protein, 205 aa, human
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 51 - protein, 5 aa, Artificial Sequence
  Peptide linker G4S
GGGGS

SEQ ID NO: 52 - protein, 10 aa, Artificial Sequence
  Peptide linker (G4S)2
GGGGSGGGGS

SEQ ID NO: 53 - protein, 10 aa, Artificial Sequence
  Peptide linker (SG4)2
SGGGGSGGGG

SEQ ID NO: 54 - protein, 15 aa, Artificial Sequence
  Peptide linker (G4S)3
GGGGSGGGGSGGGGS

SEQ ID NO: 55 - protein, 14 aa, Artificial Sequence
  Peptide linker G4(SG4)2
GGGGSGGGGSGGGG

SEQ ID NO: 56 - protein, 20 aa, Artificial Sequence
  Peptide linker (G4S)4
GGGGSGGGGSGGGGSGGGGS

SEQ ID NO: 57 - protein, 10 aa, Artificial Sequence
  Peptide linker
GSPGSSSSGS

SEQ ID NO: 58 - protein, 8 aa, Artificial Sequence
  Peptide linker
GSGSGSGS

SEQ ID NO: 59 - protein, 8 aa, Artificial Sequence
  Peptide linker
GSGSGNGS

SEQ ID NO: 60 - protein, 8 aa, Artificial Sequence
  Peptide linker
GGSGSGSG

SEQ ID NO: 61 - protein, 6 aa, Artificial Sequence
  Peptide linker
GGSGSG

SEQ ID NO: 62 - protein, 4 aa, Artificial Sequence
  Peptide linker
GGSG

SEQ ID NO: 63 - protein, 8 aa, Artificial Sequence
  Peptide linker
GGSGNGSG

SEQ ID NO: 64 - protein, 8 aa, Artificial Sequence
  Peptide linker
GGNGSGSG

SEQ ID NO: 65 - protein, 6 aa, Artificial Sequence
  Peptide linker
GGNGSG

SEQ ID NO: 66 - protein, 501 aa, human
MAAASVTPPGSLELLQPGFSKTLLGTKLEAKYLCSACRNVLRRPFQAQCGHRYCSFCLASILSSGPQNCAACVHEGIYEEGISILESSSAFPDNAARREVESLPAVCPSDGCTWKGTLKEYESCHEGRCPLMLTECPACKGLVRLGEKERHLEHECPERSLSCRHCRAPCCGADVKAHHEVCPKFPLTCDGCGKKKIPREKFQDHVKTCGKCRVPCRFHAIGCLETVEGEKQQEHEVQWLREHLAMLLSSVLEAKPLLGDQSHAGSELLQRCESLEKKTATFENIVCVLNREVERVAMTAEACSRQHRLDQDKIEALSSKVQQLERSIGLKDLAMADLEQKVLEMEASTYDGVFIWKISDFARKRQEAVAGRIPAIFSPAFYTSRYGYKMCLRIYLNGDGTGRGTHLSLFFVVMKGPNDALLRWPFNQKVTLMLLDQNNREHVIDAFRPDVTSSSFQRPVNDMNIASGCPLFCPVSKMEAKNSYVRDDAIFIKAIVDLTGL

SEQ ID NO: 67 - protein, 1170 aa, human
MGLAWGLGVLFLMHVCGTNRIPESGGDNSVFDIFELTGAARKGSGRRLVKGPDPSSPAFRIEDANLIPPVPDDKFQDLVDAVRAEKGFLLLASLRQMKKTRGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTSVLLTLDNNVVNGSSPAIRTNYIGHKTKDLQAICGISCDELSSMVLELRGLRTIVTTLQDSIRKVTEENKELANELRRPPLCYHNGVQYRNNEEWTVDSCTECHCQNSVTICKKVSCPIMPCSNATVPDGECCPRCWPSDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPINGGWGPWSPWDICSVTCGGGVQKRSRLCNNPTPQFGGKDCVGDVTENQICNKQDCPIDGCLSNPCFAGVKCTSYPDGSWKCGACPPGYSGNGIQCTDVDECKEVPDACFNHNGEHRCENTDPGYNCLPCPPRFTGSQPFGQGVEHATANKQVCKPRNPCTDGTHDCNKNAKCNYLGHYSDPMYRCECKPGYAGNGIICGEDTDLDGWPNENLVCVANATYHCKKDNCPNLPNSGQEDYDKDGIGDACDDDDDNDKIPDDRDNCPFHYNPAQYDYDRDDVGDRCDNCPYNHNPDQADTDNNGEGDACAADIDGDGILNERDNCQYVYNVDQRDTDMDGVGDQCDNCPLEHNPDQLDSDSDRIGDTCDNNQDIDEDGHQNNLDNCPYVPNANQADHDKDGKGDACDHDDDNDGIPDDKDNCRLVPNPDQKDSDGDGRGDACKDDFDHDSVPDIDDICPENVDISETDFRRFQMIPLDPKGTSQNDPNWVVRHQGKELVQTVNCDPGLAVGYDEFNAVDFSGTFFINTERDDDYAGFVFGYQSSSRFYVVMWKQVTQSYWDTNPTRAQGYSGLSVKVVNSTTGPGEHLRNALWHTGNTPGQVRTLWHDPRHIGWKDFTAYRWRLSHRPKTGFIRVVMYEGKKIMADSGPIYDKTYAGGRLGLFVFSQEMVFFSDLKYECRDP

SEQ ID NO: 68 - protein, 622 aa, human
MRGLLCWPVLLLLLQPWETQLQLTGPRCHTGPLDLVFVIDSSRSVRPFEFETMRQFLMGLLRGLNVGPNATRVGVIQYSSQVQSVFPLRAFSRREDMERAIRDLVPLAQGTMTGLAIQYAMNVAFSVAEGARPPEERVPRVAVIVTDGRPQDRVAEVAAQARARGIEIYAVGVQRADVGSLRAMASPPLDEHVFLVESFDLIQEFGLQFQSRLCGKDQCAEGGHGCQHQCVNAWAMFHCTCNPGYKLAADNKSCLAIDLCAEGTHGCEHHCVNSPGSYFCHCQVGFVLQQDQRSCRAIDYCSFGNHSCQHECVSTPGGPRCHCREGHDLQPDGRSCQVRDLCNGVDHGCEFQCVSEGLSYRCLCPEGRQLQADGKSCNRCREGHVDLVLLVDGSKSVRPQNFELVKRFVNQIVDFLDVSPEGTRVGLVQFSSRVRTEFPLGRYGTAAEVKQAVLAVEYMERGTMTGLALRHMVEHSFSEAQGARPRALNVPRVGLVFTDGRSQDDISVWAARAKEEGIVMYAVGVGKAVEAELREIASEPAELHVSYAPDFGTMTHLLENLRGSICPEEGISAGTELRSPCECESLVEFQGRTLGALESLTLNLAQLTARLEDLENQLANQK

SEQ ID NO: 69 - protein, 3623 aa, human
MMNMSLPFLWSLLTLLIFAEVNGEAGELELQRQKRSINLQQPRMATERGNLVFLTGSAQNIEFRTGSLGKIKLNDEDLSECLHQIQKNKEDIIELKGSAIGLPQNISSQIYQLNSKLVDLERKFQGLQQTVDKKVCSSNPCQNGGTCLNLHDSFFCICPPQWKGPLCSADVNECEIYSGTPLSCQNGGTCVNTMGSYSCHCPPETYGPQCASKYDDCEGGSVARCVHGICEDLMREQAGEPKYSCVCDAGWMFSPNSPACTLDRDECSFQPGPCSTLVQCFNTQGSFYCGACPTGWQGNGYICEDINECEINNGGCSVAPPVECVNTPGSSHCQACPPGYQGDGRVCTLTDICSVSNGGCHPDASCSSTLGSLPLCTCLPGYTGNGYGPNGCVQLSNICLSHPCLNGQCIDTVSGYFCKCDSGWTGVNCTENINECLSNPCLNGGTCVDGVDSFSCECTRLWTGALCQVPQQVCGESLSGINGSFSYRSPDVGYVHDVNCFWVIKTEMGKVLRITFTFFRLESMDNCPHEFLQVYDGDSSSAFQLGRFCGSSLPHELLSSDNALYFHLYSEHLRNGRGFTVRWETQQPECGGILTGPYGSIKSPGYPGNYPPGRDCVWIVVTSPDLLVTFTFGTLSLEHHDDCNKDYLEIRDGPLYQDPLLGKFCTTFSVPPLQTTGPFARIHFHSDSQISDQGFHITYLTSPSDLRCGGNYTDPEGELFLPELSGPFTHTRQCVYMMKQPQGEQIQINFTHVELQCQSDSSQNYIEVRDGETLLGKVCGNGTISHIKSITNSVWIRFKIDASVEKASFRAVYQVACGDELTGEGVIRSPFFPNVYPGERTCRWTIHQPQSQVILLNFTVFEIGSSAHCETDYVEIGSSSILGSPENKKYCGTDIPSFITSVYNFLYVTFVKSSSTENHGFMAKFSAEDLACGEILTESTGTIQSPGHPNVYPHGINCTWHILVQPNHLIHLMFETFHLEFHYNCTNDYLEVYDTDSETSLGRYCGKSIPPSLTSSGNSLMLVFVTDSDLAYEGFLINYEAISAATACLQDYTDDLGTFTSPNFPNNYPNNWECIYRITVRTGQLIAVHFTNFSLEEAIGNYYTDFLEIRDGGYEKSPLLGIFYGSNLPPTIISHSNKLWLKFKSDQIDTRSGFSAYWDGSSTGCGGNLTTSSGTFISPNYPMPYYHSSECYWWLKSSHGSAFELEFKDFHLEHHPNCTLDYLAVYDGPSSNSHLLTQLCGDEKPPLIRSSGDSMFIKLRTDEGQQGRGFKAEYRQTCENVVIVNQTYGILESIGYPNPYSENQHCNWTIRATTGNTVNYTFLAFDLEHHINCSTDYLELYDGPRQMGRYCGVDLPPPGSTTSSKLQVLLLTDGVGRREKGFQMQWFVYGCGGELSGATGSFSSPGFPNRYPPNKECIWYIRTDPGSSIQLTIHDFDVEYHSRCNFDVLEIYGGPDFHSPRIAQLCTQRSPENPMQVSSTGNELAIRFKTDLSINGRGFNASWQAVTGGCGGIFQAPSGEIHSPNYPSPYRSNTDCSWVIRVDRNHRVLLNFTDFDLEPQDSCIMAYDGLSSTMSRLARTCGREQLANPIVSSGNSLFLRFQSGPSRQNRGFRAQFRQACGGHILTSSFDTVSSPRFPANYPNNQNCSWIIQAQPPLNHITLSFTHFELERSTTCARDFVEILDGGHEDAPLRGRYCGTDMPHPITSFSSALTLRFVSDSSISAGGFHTTVTASVSACGGTFYMAEGIFNSPGYPDIYPPNVECVWNIVSSPGNRLQLSFISFQLEDSQDCSRDFVEIREGNATGHLVGRYCGNSFPLNYSSIVGHTLWVRFISDGSGSGTGFQATFMKIFGNDNIVGTHGKVASPFWPENYPHNSNYQWTVNVNASHVVHGRILEMDIEEIQNCYYDKLRIYDGPSIHARLIGAYCGTQTESFSSTGNSLTFHFYSDSSISGKGFLLEWFAVDAPDGVLPTIAPGACGGFLRTGDAPVFLFSPGWPDSYSNRVDCTWLIQAPDSTVELNILSLDIESHRTCAYDSLVIRDGDNNLAQQLAVLCGREIPGPIRSTGEYMFIRFTSDSSVTRAGFNASFHKSCGGYLHADRGIITSPKYPETYPSNLNCSWHVLVQSGLTIAVHFEQPFQIPNGDSSCNQGDYLVLRNGPDICSPPLGPPGGNGHFCGSHASSTLFTSDNQMFVQFISDHSNEGQGFKIKYEAKSLACGGNVYIHDADSAGYVTSPNHPHNYPPHADCIWILAAPPETRIQLQFEDRFDIEVTPNCTSNYLELRDGVDSDAPILSKFCGTSLPSSQWSSGEVMYLRFRSDNSPTHVGFKAKYSIAQCGGRVPGQSGVVESIGHPTLPYRDNLFCEWHLQGLSGHYLTISFEDFNLQNSSGCEKDFVEIWDNHTSGNILGRYCGNTIPDSIDTSSNTAVVRFVTDGSVTASGFRLRFESSMEECGGDLQGSIGTFTSPNYPNPNPHGRICEWRITAPEGRRITLMFNNLRLATHPSCNNEHVIVFNGIRSNSPQLEKLCSSVNVSNEIKSSGNTMKVIFFTDGSRPYGGFTASYTSSEDAVCGGSLPNTPEGNFTSPGYDGVRNYSRNLNCEWTLSNPNQGNSSISIHFEDFYLESHQDCQFDVLEFRVGDADGPLMWRLCGPSKPTLPLVIPYSQVWIHFVTNERVEHIGFHAKYSFTDCGGIQIGDSGVITSPNYPNAYDSLTHCSSLLEAPQGHTITLTFSDFDIEPHTTCAWDSVTVRNGGSPESPIIGQYCGNSNPRTIQSGSNQLVVTFNSDHSLQGGGFYATWNTQTLGCGGIFHSDNGTIRSPHWPQNFPENSRCSWTAITHKSKHLEISFDNNFLIPSGDGQCQNSFVKVWAGTEEVDKALLATGCGNVAPGPVITPSNTFTAVFQSQEAPAQGFSASFVSRCGSNFTGPSGYIISPNYPKQYDNNMNCTYVIEANPLSVVLLTFVSFHLEARSAVTGSCVNDGVHIIRGYSVMSTPFATVCGDEMPAPLTIAGPVLLNFYSNEQITDFGFKFSYRIISCGGVFNFSSGIITSPAYSYADYPNDMHCLYTITVSDDKVIELKFSDFDVVPSTSCSHDYLAIYDGANTSDPLLGKFCGSKRPPNVKSSNNSMLLVFKTDSFQTAKGWKMSFRQTLGPQQGCGGYLTGSNNTFASPDSDSNGMYDKNLNCVWIIIAPVNKVIHLTFNTFALEAASTRQRCLYDYVKLYDGDSENANLAGTFCGSTVPAPFISSGNFLTVQFISDLTLEREGFNATYTIMDMPCGGTYNATWTPQNISSPNSSDPDVPFSICTWVIDSPPHQQVKITVWALQLTSQDCTQNYLQLQDSPQGHGNSRFQFCGRNASAVPVFYSSMSTAMVIFKSGVVNRNSRMSFTYQIADCNRDYHKAFGNLRSPGWPDNYDNDKDCTVTLTAPQNHTISLFFHSLGIENSVECRNDFLEVRNGSNSNSPLLGKYCGTLLPNPVFSQNNELYLRFKSDSVTSDRGYEIIWTSSPSGCGGTLYGDRGSFTSPGYPGTYPNNTYCEWVLVAPAGRLVTINFYFISIDDPGDCVQNYLTLYDGPNASSPSSGPYCGGDTSIAPFVASSNQVFIKFHADYARRPSAFRLTWDS

SEQ ID NO: 70 - protein, 32 aa, Artificial Sequence
  isoleucine zipper domain
IKQIEDKIEEILSKIYHIENEIARIKKLIGER

SEQ ID NO: 71 - protein, 478 aa, Artificial Sequence
  FAP(28H1)(VHCH1)-CMPtd-4-1BBL(71-254)
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEEDPCACESLVKFQAKVEGLLQALTRKLEAVSKRLAILENTVVSGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 72 - protein, 427 aa, Artificial Sequence
  FAP(VHCH1)-CMPtd-Ox40L(51-183)
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEEDPCACESLVKFQAKVEGLLQALTRKLEAVSKRLAILENTVVSGGGGSGGGGSGGGGSGGGGSQVSHRYPRIQSIKVQFTEYKKEKGFILTSQKEDEIMKVQDNSVIINCDGFYLISLKGYFSQEVDISLHYQKDEEPLFQLKKVRSVNSLMVASLTYKDKVYLNVTTDNTSLDDFHVNGGELILIHQNPGEFCVL

SEQ ID NO: 73 - DNA, 1434 bp, Artificial Sequence
  nucleotide sequence of FAP(28H1)(VHCH1)-CMPtd-4-1BBL(71-254)
gaagtgcagctgctggaatccggcggaggcctggtgcagcctggcggatctctgagactgtcctgcgccgcctccggcttcaccttctcctcccacgccatgtcctgggtccgacaggctcctggcaaaggcctggaatgggtgtccgccatctgggcctccggcgagcagtactacgccgactctgtgaagggccggttcaccatctcccgggacaactccaagaacaccctgtacctgcagatgaactccctgcgggccgaggacaccgccgtgtactactgtgccaagggctggctgggcaacttcgactactggggacagggcaccctggtcaccgtgtccagcgctagcacaaagggacctagcgtgttccccctggcccccagcagcaagtctacatctggcggaacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagctctctgggcacccagacctacatctgcaacgtgaaccacaagcccagcaacaccaaggtggacaagaaggtggaacccaagagctgcgacggcggagggggatctggcggcggaggatccgaggaagatccttgcgcctgcgagagcctcgtgaagttccaggccaaggtggaaggactgctgcaggccctgacccggaaactggaagccgtgtccaagcggctggccatcctggaaaacaccgtggtgtccggaggcggaggatctggcggcggaggaagtggcggaggcggatctggcggcggaggatctagagagggacccgaactgtcccctgacgatccagccgggctgctggatctgagacagggaatgttcgcccagctggtggctcagaatgtgctgctgattgacggacctctgagctggtactccgacccagggctggcaggggtgtccctgactgggggactgtcctacaaagaagatacaaaagaactggtggtggctaaagctggggtgtactatgtgttttttcagctggaactgaggcgggtggtggctggggagggctcaggatctgtgtccctggctctgcatctgcagccactgcgctctgctgctggcgcagctgcactggctctgactgtggacctgccaccagcctctagcgaggccagaaacagcgccttcgggttccaaggacgcctgctgcatctgagcgccggacagcgcctgggagtgcatctgcatactgaagccagagcccggcatgcttggcagctgactcagggggcaactgtgctgggactgtttcgcgtgacacctgagatccctgccggactgccaagccctagatcagaa

SEQ ID NO: 74 - DNA, 1281 bp, Artificial Sequence
  nucleotide sequence of FAP(28H1)(VHCH1)-CMPtd-Ox40L(51-183)
gaagtgcagctgctggaatccggcggaggcctggtgcagcctggcggatctctgagactgtcctgcgccgcctccggcttcaccttctcctcccacgccatgtcctgggtccgacaggctcctggcaaaggcctggaatgggtgtccgccatctgggcctccggcgagcagtactacgccgactctgtgaagggccggttcaccatctcccgggacaactccaagaacaccctgtacctgcagatgaactccctgcgggccgaggacaccgccgtgtactactgtgccaagggctggctgggcaacttcgactactggggacagggcaccctggtcaccgtgtccagcgctagcacaaagggacctagcgtgttccccctggcccccagcagcaagtctacatctggcggaacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagctctctgggcacccagacctacatctgcaacgtgaaccacaagcccagcaacaccaaggtggacaagaaggtggaacccaagagctgcgacggcggagggggatctggcggcggaggatccgaggaagatccttgcgcctgcgagagcctcgtgaagttccaggccaaggtggaaggactgctgcaggccctgacccggaaactggaagccgtgtccaagcggctggccatcctggaaaacaccgtggtgtccggaggcggaggatctggcggcggaggaagtggcggagggggatctgggggaggcggatctcaggtgtcccacagatacccccggatccagagcatcaaggtgcagttcaccgagtacaagaaagagaagggcttcatcctgaccagccagaaagaggacgagatcatgaaggtgcaggacaacagcgtgatcatcaactgcgacggcttctacctgatcagcctgaagggctacttcagccaggaagtggacatcagcctgcactaccagaaggacgaggaacccctgttccagctgaagaaagtgcggagcgtgaacagcctgatggtggccagcctgacctacaaggacaaggtgtacctgaacgtgaccaccgacaacaccagcctggacgacttccacgtgaacggcggcgagctgatcctgattcaccagaaccccggcgagttctgcgtgctc

SEQ ID NO: 75 - DNA, 645 bp, Artificial Sequence
  nucleotide sequence of Antianti-FAP(28H1) light chain
gagatcgtgctgacccagtcccccggcaccctgtctctgagccctggcgagagagccaccctgtcctgcagagcctcccagtccgtgtcccggtcctacctcgcctggtatcagcagaagcccggccaggcccctcggctgctgatcatcggcgcctctaccagagccaccggcatccctgaccggttctccggctctggctccggcaccgacttcaccctgaccatctcccggctggaacccgaggacttcgccgtgtactactgccagcagggccaggtcatccctcccacctttggccagggcaccaaggtggaaatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 76 - protein, 215 aa, Artificial Sequence
  Anti-FAP(28H1) light chain
EIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - DNA, 1431 bp, Artificial Sequence
  nucleotide sequence of DP47 (VHCH1)-CMPtd-4-1BBL(71-254)
gaggtgcaattgttggagtctgggggaggcttggtacagcctggggggtccctgagactctcctgtgcagcctccggattcacctttagcagttatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcagatgaacagcctgagagccgaggacacggccgtatattactgtgcgaaaggcagcggatttgactactggggccaaggaaccctggtcaccgtctcgagtgctagcacaaagggacctagcgtgttccccctggcccccagcagcaagtctacatctggcggaacagccgccctgggctgcctcgtgaaggactactttcccgagcccgtgaccgtgtcctggaactctggcgctctgacaagcggcgtgcacacctttccagccgtgctgcagagcagcggcctgtactctctgagcagcgtcgtgacagtgcccagcagctctctgggcacccagacctacatctgcaacgtgaaccacaagcccagcaacaccaaggtggacaagaaggtggaacccaagagctgcgacggcggagggggatctggcggcggaggatccgaggaagatccttgcgcctgcgagagcctcgtgaagttccaggccaaggtggaaggactgctgcaggccctgacccggaaactggaagccgtgtccaagcggctggccatcctggaaaacaccgtggtgtccggaggcggaggatctggcggcggaggaagtggcggaggcggatctggcggcggaggatctagagagggacccgaactgtcccctgacgatccagccgggctgctggatctgagacagggaatgttcgcccagctggtggctcagaatgtgctgctgattgacggacctctgagctggtactccgacccagggctggcaggggtgtccctgactgggggactgtcctacaaagaagatacaaaagaactggtggtggctaaagctggggtgtactatgtgttttttcagctggaactgaggcgggtggtggctggggagggctcaggatctgtgtccctggctctgcatctgcagccactgcgctctgctgctggcgcagctgcactggctctgactgtggacctgccaccagcctctagcgaggccagaaacagcgccttcgggttccaaggacgcctgctgcatctgagcgccggacagcgcctgggagtgcatctgcatactgaagccagagcccggcatgcttggcagctgactcagggggcaactgtgctgggactgtttcgcgtgacacctgagatccctgccggactgccaagccctagatcagaa

SEQ ID NO: 78 - DNA, 645 bp, Artificial Sequence
  nucleotide sequence of DP47 light chain
gaaatcgtgttaacgcagtctccaggcaccctgtctttgtctccaggggaaagagccaccctctcttgcagggccagtcagagtgttagcagcagctacttagcctggtaccagcagaaacctggccaggctcccaggctcctcatctatggagcatccagcagggccactggcatcccagacaggttcagtggcagtggatccgggacagacttcactctcaccatcagcagactggagcctgaagattttgcagtgtattactgtcagcagtatggtagctcaccgctgacgttcggccaggggaccaaagtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 79 - protein, 477 aa, Artificial Sequence
  DP47(VHCH1)-CMPtd-4-1BBL(71-254)
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEEDPCACESLVKFQAKVEGLLQALTRKLEAVSKRLAILENTVVSGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 80 - protein, 215 aa, Artificial Sequence
  DP47 light chain
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - DNA, 1335 bp, Artificial Sequence
  nucleotide sequence of DP47 heavy chain (hu IgG1 PGLALA)
gaggtgcaattgttggagtctgggggaggcttggtacagcctggggggtccctgagactctcctgtgcagcctccggattcacctttagcagttatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcagatgaacagcctgagagccgaggacacggccgtatattactgtgcgaaaggcagcggatttgactactggggccaaggaaccctggtcaccgtctcgagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 82 - protein, 445 aa, Artificial Sequence
  DP47 heavy chain (hu IgG1 PGLALA)
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 163 aa, Homo sapiens
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQ

SEQ ID NO: 84 - protein, 163 aa, cynomolgus
LQDLCSNCPAGTFCDNNRSQICSPCPPNSFSSAGGQRTCDICRQCKGVFKTRKECSSTSNAECDCISGYHCLGAECSMCEQDCKQGQELTKKGCKDCCFGTFNDQKRGICRPWTNCSLDGKSVLVNGTKERDVVCGPSPADLSPGASSATPPAPAREPGHSPQ

SEQ ID NO: 85 - protein, 164 aa, Mus musculus
VQNSCDNCQPGTFCRKYNPVCKSCPPSTFSSIGGQPNCNICRVCAGYFRFKKFCSSTHNAECECIEGFHCLGPQCTRCEKDCRPGQELTKQGCKTCSLGTFNDQNGTGVCRPWTNCSLDGRSVLKTGTTEKDVVCGPPVVSFSPSTTISVTPEGGPGGHSLQVL

SEQ ID NO: 86 - DNA, 681 bp, Artificial sequence
  Fc hole chain
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctctcgtgcgcagtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 87 - DNA, 1266 bp, Artificial sequence
  human 4-1BB Fc knob chain
ctgcaggacccctgcagcaactgccctgccggcaccttctgcgacaacaaccggaaccagatctgcagcccctgcccccccaacagcttcagctctgccggcggacagcggacctgcgacatctgcagacagtgcaagggcgtgttcagaacccggaaagagtgcagcagcaccagcaacgccgagtgcgactgcacccccggcttccattgtctgggagccggctgcagcatgtgcgagcaggactgcaagcagggccaggaactgaccaagaagggctgcaaggactgctgcttcggcaccttcaacgaccagaagcggggcatctgccggccctggaccaactgtagcctggacggcaagagcgtgctggtcaacggcaccaaagaacgggacgtcgtgtgcggccccagccctgctgatctgtctcctggggccagcagcgtgacccctcctgcccctgccagagagcctggccactctcctcaggtcgacgaacagttatattttcagggcggctcacccaaatctgcagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatccggaggcctgaacgacatcttcgaggcccagaagattgaatggcacgag

SEQ ID NO: 88 - DNA, 1266 bp, Artificial sequence
  cynomolgus 4-1BB Fc knob chain
ttgcaggatctgtgtagtaactgcccagctggtacattctgtgataataacaggagtcagatttgcagtccctgtcctccaaatagtttctccagcgcaggtggacaaaggacctgtgacatatgcaggcagtgtaaaggtgttttcaagaccaggaaggagtgttcctccaccagcaatgcagagtgtgactgcatttcagggtatcactgcctgggggcagagtgcagcatgtgtgaacaggattgtaaacaaggtcaagaattgacaaaaaaaggttgtaaagactgttgctttgggacatttaatgaccagaaacgtggcatctgtcgcccctggacaaactgttctttggatggaaagtctgtgcttgtgaatgggacgaaggagagggacgtggtctgcggaccatctccagccgacctctctccaggagcatcctctgcgaccccgcctgcccctgcgagagagccaggacactctccgcaggtcgacgaacagttatattttcagggcggctcacccaaatctgcagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatccggaggcctgaacgacatcttcgaggcccagaagattgaatggcacgag

SEQ ID NO: 89 - DNA, 1269 bp, Artificial sequence
  Murine 4-1BB Fc knob chain
gtgcagaacagctgcgacaactgccagcccggcaccttctgccggaagtacaaccccgtgtgcaagagctgcccccccagcaccttcagcagcatcggcggccagcccaactgcaacatctgcagagtgtgcgccggctacttccggttcaagaagttctgcagcagcacccacaacgccgagtgcgagtgcatcgagggcttccactgcctgggcccccagtgcaccagatgcgagaaggactgcagacccggccaggaactgaccaagcagggctgtaagacctgcagcctgggcaccttcaacgaccagaacgggaccggcgtgtgccggccttggaccaattgcagcctggacgggagaagcgtgctgaaaaccggcaccaccgagaaggacgtcgtgtgcggccctcccgtggtgtccttcagccctagcaccaccatcagcgtgacccctgaaggcggccctggcggacactctctgcaggtcctggtcgacgaacagttatattttcagggcggctcacccaaatctgcagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatccggaggcctgaacgacatcttcgaggcccagaagattgaatggcacgag

SEQ ID NO: 90 - protein, 227 aa, Artificial sequence
  Fc hole chain
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 91 - protein, 422 aa, Artificial sequence
  Human 4-1BB Fc knob chain
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQVDEQLYFQGGSPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGLNDIFEAQKIEWHE

SEQ ID NO: 92 - protein, 422 aa, Artificial sequence
  Cynomolgus 4-1BB Fc knob chain
LQDLCSNCPAGTFCDNNRSQICSPCPPNSFSSAGGQRTCDICRQCKGVFKTRKECSSTSNAECDCISGYHCLGAECSMCEQDCKQGQELTKKGCKDCCFGTFNDQKRGICRPWTNCSLDGKSVLVNGTKERDVVCGPSPADLSPGASSATPPAPAREPGHSPQVDEQLYFQGGSPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGLNDIFEAQKIEWHE

SEQ ID NO: 93 - protein, 423 aa, Artificial sequence
  Murine 4-1BB Fc knob chain
VQNSCDNCQPGTFCRKYNPVCKSCPPSTFSSIGGQPNCNICRVCAGYFRFKKFCSSTHNAECECIEGFHCLGPQCTRCEKDCRPGQELTKQGCKTCSLGTFNDQNGTGVCRPWTNCSLDGRSVLKTGTTEKDVVCGPPVVSFSPSTTISVTPEGGPGGHSLQVLVDEQLYFQGGSPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGLNDIFEAQKIEWHE